(19) United States
(12) Patent Application Publication
(10) Pub. No.: US 2004/0147502 A1
(43) Pub. Date: Jul. 29, 2004

(54) Beta lactam compounds and their use as inhibitors of tryptase

(76) Inventors: Gregory S. Bisacchi, Ringoes, NJ (US); James C. Sutton, Princeton Junction, NJ (US); William A. Slusarchyk, Skillman, NJ (US); Uwe Treuner, Nittendorf (DE); Guohua Zhao, Princeton, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/728,276

(22) Filed: Dec. 4, 2003

Related U.S. Application Data
(60) Provisional application No. 60/434,060, filed on Dec. 17, 2002.

(51) Int. Cl. A61K 31/397; C 07D 2 5/02

(52) U.S. Cl. 514/210.02; 540/200

(57) ABSTRACT

Beta lactam compounds are provided which have the structure

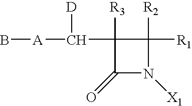

wherein B, A, D, R₁, R₂, R₃ and X₁ are as defined herein, and which are useful as inhibitors of tryptase, thrombin, trypsin, Factor Xa, Factor VIIa, and urokinase-type plasminogen activator and may be employed in preventing and/or treating asthma and allergic rhinitis.

This application claims the priority benefit of U.S. Provisional Application No. 60/434,060 filed Dec. 17, 2002, the disclosure of which is incorporated herein by reference in its entirety.

### FIELD OF THE INVENTION

The present invention relates to beta lactam compounds which are inhibitors of tryptase and to a method for preventing or treating asthma and chronic rhinitis employing such compounds.

### BACKGROUND OF THE INVENTION

Han in U.S. Pat. Nos. 5,037,819, 5,110,812, 5,175,283, 5,250,677 and 5,326,863 discloses 3-guanidinoalkyl-2-azetidinones of the formula

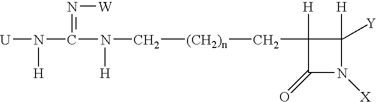

wherein:

U and W are independently selected from hydrogen and amino protecting groups;

n is an integer from 1 to 3;

X is hydrogen, trialkylsilyl, arylsulfonyl, amino substituted arylsulfonyl, alkylsulfonyl, arylaminocarbonyl, alkylcarbonyl or arylcarbonyl;

Y is hydrogen, arylalkenyl, arylalkyl, formyl, carboxy, alkoxycarbonyl, acyloxy, arylthio, arylsulfinyl, arylsulfonyl, alkythio, alkylsulfinyl, alkylsulfonyl, arylaminocarbonyl,

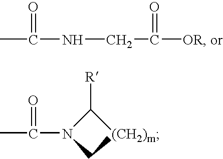

R is hydrogen, alkyl, or arylalkyl;

m is an integer from 1 to 3; and

R′ is hydrogen or —CO₂R″ wherein R″ is hydrogen, alkyl, or arylalkyl.

Han further discloses that the above compounds wherein:

U and W are hydrogen;

X is arylsulfonyl, amino substituted arylsulfonyl, alkylsulfonyl, arylaminocarbonyl, alkylcarbonyl, or arylcarbonyl; and

Y is hydrogen, arylalkyl, carboxy, alkoxycarbonyl, acyloxy, arylsulfonyl, alkylthio, alkylsulfonyl, arylaminocarbonyl,

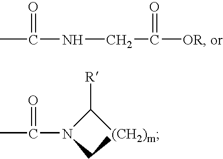

R is hydrogen, alkyl or arylalkyl;

R′ is hydrogen or —CO₂R″;

R″ is hydrogen, alkyl, or arylalkyl and pharmaceutically acceptable salts thereof are inhibitors against serine proteases, particularly against thrombin and trypsin, and can be used to control blood coagulation or to treat pancreatitis.

Han defines “aryl” as a phenyl or naphthyl group which may be unsubstituted or substituted with one or more groups such as amino, nitro, or alkyl and defines “amino” as unsubstituted or substituted with one or two alkyl radicals.

U.S. Pat. No. 6,335,324 to Bisacchi et al. discloses compounds of the following formula:

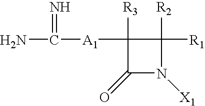

wherein:

R₁ is hydrogen, carboxy, alkoxycarbonyl, A₂-aryl,

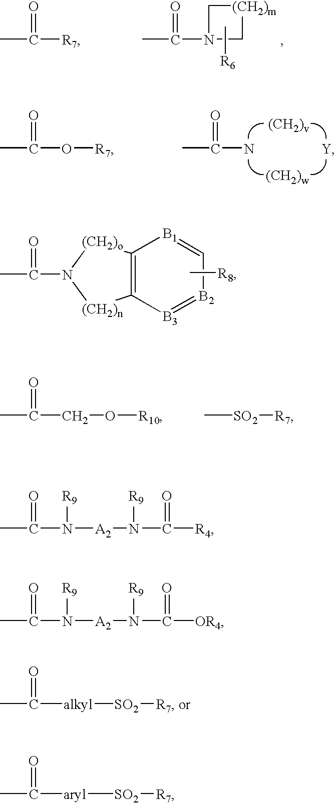

or R₁ is alkyl provided that R₂ is alkyl and R₃ is hydrogen;

R₂ and R₃ are both hydrogen, or R₂ is alkyl provided that R₃ is hydrogen, or R₃ is alkyl provided that R₂ is hydrogen;

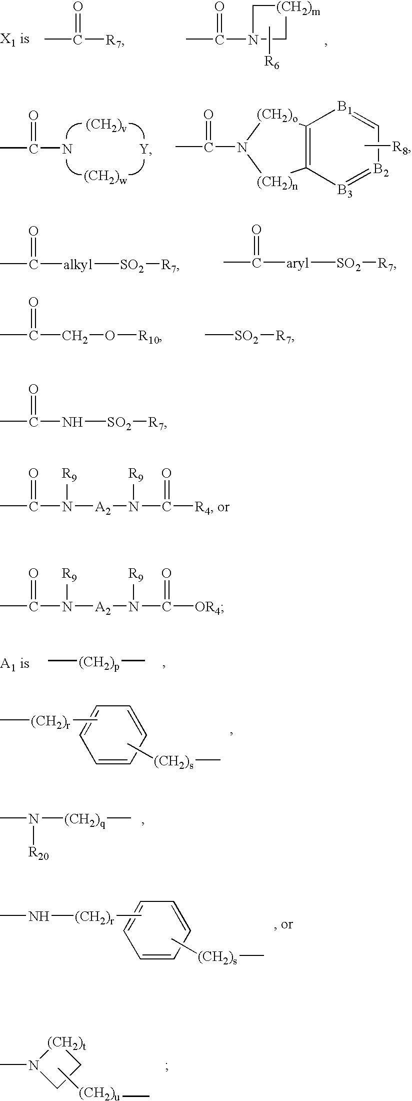

R₄ and R₅ are independently selected from hydrogen, alkyl, substituted alkyl, cycloalkyl, substituted cycloalkyl, A₂-cycloalkyl, A₂-substituted cycloalkyl, aryl, substituted aryl, A₂-aryl, A₂-substituted aryl, heteroaryl, A₂-heteroaryl, heterocycloalkyl, A₂-heterocycloalkyl, aryl-A₃-aryl, A₂-aryl-A₃-aryl, aryl-A₃-cycloalkyl, A₂-aryl-A₃-cycloalkyl, aryl-A₃-heteroaryl, A₂-aryl-A₃-heteroaryl, aryl-A₃-heterocycloalkyl, A₂-aryl-A₃-heterocycloalkyl, aryl-A₃-substituted aryl, A₂-aryl-A₃-substitued aryl, aryl-A₃-substituted cycloalkyl, A₂-aryl-A₃-substituted cycloalkyl, cycloalkyl-A₃-cycloalkyl, A₂-cycloalkyl-A₃-cycloalkyl, cycloalkyl-A₃-aryl, A₂-cycloalkyl-A₃-aryl, cycloalkyl-A₃-heteroaryl, A₂-cycloalkyl-A₃-heteroaryl, cycloalkyl-A₃-heterocycloalkyl, A₂-cycloalkyl-A₃-heterocycloalkyl, cycloalkyl-A₃-substituted cycloalkyl, A₂-cycloalkyl-A₃-substituted cycloalkyl, cycloalkyl-A₃-substituted aryl, A₂-cycloalkyl-A₃-substituted aryl, substituted cycloalkyl-A₃-cycloalkyl, A₂-substituted cycloalkyl-A₃-cycloalkyl, substituted cycloalkyl-A₃-substituted cycloalkyl, A₂-substituted cycloalkyl-A₃-substituted cycloalkyl, substituted cycloalkyl-A₃-aryl, A₂-substituted cycloalkyl-A₃-aryl, substituted cycloalkyl-A₃-heteroaryl, A₂-substituted cycloalkyl-A₃-heteroaryl, substituted cycloalkyl-A₃-heterocycloalkyl, A₂-substituted cycloalkyl-A₃-heterocycloalkyl, substituted cycloalkyl-A₃-substituted aryl, A₂-substituted cycloalkyl-A₃-substituted aryl, heteroaryl-A₃-heteroaryl, A₂-heteroaryl-A₃-heteroaryl, heteroaryl-A₃-cycloalkyl, A₂-heteroaryl-A₃-cycloalkyl, heteroaryl-A₃-substituted cycloalkyl, A₂-heteroaryl-A₃-substituted cycloalkyl, heteroaryl-A₃-aryl, A₂-heteroaryl-A₃-aryl, heteroaryl-A₃-heterocycloalkyl, A₂-heteroaryl-A₃-heterocycloalkyl, heteroaryl-A₃-substituted aryl, A₂-heteroaryl-A₃-substituted aryl, heterocycloalkyl-A₃-heterocycloalkyl, A₂-heterocycloalkyl-A₃-heterocycloalkyl, heterocycloalkyl-A₃-cycloalkyl, A₂-heterocycloalkyl-A₃-cycloalkyl, heterocycloalkyl-A₃-substitute d cycloalkyl, A₂-heterocycloalkyl-A₃-substituted cycloalkyl, heterocycloalkyl-A₃-aryl, A₂-heterocycloalkyl-A₃-aryl, heterocycloalkyl-A₃-substituted aryl, A₂-heterocycloalkyl-A₃-substituted aryl, heterocycloalkyl-A₃-heteroaryl, A₂-heterocycloalkyl-A₃-heteroaryl, substituted aryl-A₃-substituted aryl, A₂-substituted aryl-A₃-substituted aryl, substituted aryl-A₃-cycloalkyl, A₂-substituted aryl-A₃-cycloalkyl, substituted aryl-A₃-substituted cycloalkyl, A₂-substituted aryl-A₃-substituted cycloalkyl, substituted aryl-A₃-aryl, A₂-substituted aryl-A₃-aryl, substituted aryl-A₃-heteroaryl, A₂-substituted aryl-A₃-heteroaryl, substituted aryl-A₃-heterocycloalkyl, and A₂-substituted aryl-A₃-heterocycloalkyl;

R₆ is hydrogen, alkyl, substituted alkyl, cycloalkyl, substituted cycloalkyl, A₂-cycloalkyl, A₂-substituted cycloalkyl, aryl, substituted aryl, A₂-aryl, A₂-substituted aryl, aryl-A₃-aryl, A₂-aryl-A₃-aryl, heteroaryl, A₂-heteroaryl, heterocycloalkyl, A₂-heterocycloalkyl, aryl-A₃-cycloalkyl, A₂-aryl-A₃-cycloalkyl, aryl-A₃-heteroaryl, A₂-aryl-A₃-heteroaryl, aryl-A₃-heterocycloalkyl, A₂-aryl-A₃-heterocycloalkyl, carboxy, alkoxycarbonyl, aryloxycarbonyl,

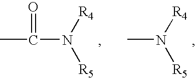

alkoxycarbonylamino, aryloxycarbonylamino, arylcarbonylamino, —N(alkyl)(alkoxycarbonyl), —N(alkyl)(aryloxycarbonyl), alkylcarbonylamino, —N(alkyl)(alkylcarbonyl), or —N(alkyl)(arylcarbonyl);

m is an integer from 1 to 5;

Y is O, S, N—R₄, N—SO₂—R₇,

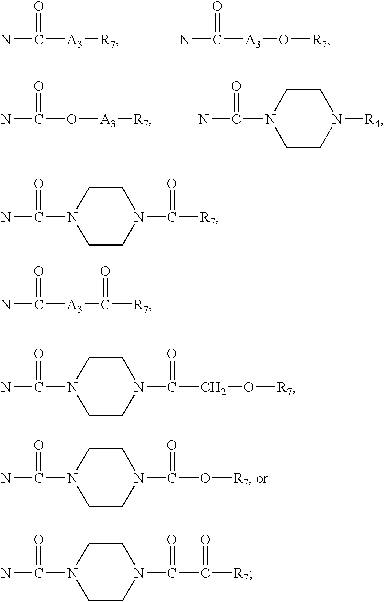

R₇ is alkyl, substituted alkyl, cycloalkyl, substituted cycloalkyl, A₂-cycloalkyl, A₂-substituted cycloalkyl, aryl, substituted aryl, A₂-aryl, A₂-substituted aryl, heteroaryl, A₂-heteroaryl, heterocycloalkyl, A₂-heterocycloalkyl, aryl-A₃-aryl, A₂-aryl-A₃-aryl, aryl-A₃-cycloalkyl, A₂-aryl-A₃-cycloalkyl, aryl-A₃-heteroaryl, A₂-aryl-A₃-heteroaryl, aryl-A₃-heterocycloalkyl, A₂-aryl-A₃-heterocycloalkyl, aryl-A₃-substituted aryl, A₂-aryl-A₃-substitued aryl, aryl-A₃-substituted cycloalkyl, A₂-aryl-A₃-substituted cycloalkyl, cycloalkyl-A₃-cycloalkyl, A₂-cycloalkyl-A₃-cycloalkyl, cycloalkyl-A₃-aryl, A₂-cycloalkyl-A₃-aryl, cycloalkyl-A₃-heteroaryl, A₂-cycloalkyl-A₃-heteroaryl, cycloalkyl-A₃-heterocycloalkyl, A₂-cycloalkyl-A₃-heterocycloalkyl, cycloalkyl-A₃-substituted cycloalkyl, A₂-cycloalkyl-A₃-substituted cycloalkyl, cycloalkyl-A₃-substituted aryl, A₂-cycloalkyl-A₃-substituted aryl, substituted cycloalkyl-A₃-cycloalkyl, A₂-substituted cycloalkyl-A₃-cycloalkyl, substituted cycloalkyl-A₃-substituted cycloalkyl, A₂-substituted cycloalkyl-A₃-substituted cycloalkyl, substituted cycloalkyl-A₃-aryl, A₂-substituted cycloalkyl-A₃-aryl, substituted cycloalkyl-A₃-heteroaryl, A₂-substituted cycloalkyl-A₃-heteroaryl, substituted cycloalkyl-A₃-heterocycloalkyl, A₂-substituted cycloalkyl-A₃-heterocycloalkyl, substituted cycloalkyl-A₃-substituted aryl, A₂-substituted cycloalkyl-A₃-substituted aryl, heteroaryl-A₃-heteroaryl, A₂-heteroaryl-A₃-heteroaryl, heteroaryl-A₃-cycloalkyl, A₂-heteroaryl-A₃-cycloalkyl, heteroaryl-A₃-substituted cycloalkyl, A₂-heteroaryl-A₃-substituted cycloalkyl, heteroaryl-A₃-aryl, A₂-heteroaryl-A₃-aryl, heteroaryl-A₃-heterocycloalkyl, A₂-heteroaryl-A₃-heterocycloalkyl, heteroaryl-A₃-substituted aryl, A₂-heteroaryl-A₃-substituted aryl, heterocycloalkyl-A₃-heterocycloalkyl, A₂-heterocycloalkyl-A₃-heterocycloalkyl, heterocycloalkyl-A₃-cycloalkyl, A₂-heterocycloalkyl-A₃-cycloalkyl, heterocycloalkyl-A₃-substituted cycloalkyl, A₂-heterocycloalkyl-A₃-substituted cycloalkyl, heterocycloalkyl-A₃-aryl, A₂-heterocycloalkyl-A₃-aryl, heterocycloalkyl-A₃-substituted aryl, A₂-heterocycloalkyl-A₃-substituted aryl, heterocycloalkyl-A₃-heteroaryl, A₂-heterocycloalkyl-A₃-heteroaryl, substituted aryl-A₃-substituted aryl, A₂-substituted aryl-A₃-substituted aryl, substituted aryl-A₃-cycloalkyl, A₂-substituted aryl-A₃-cycloalkyl, substituted aryl-A₃-substituted cycloalkyl, A₂-substituted aryl-A₃-substituted cycloalkyl, substituted aryl-A₃-aryl, A₂-substituted aryl-A₃-aryl, substituted aryl-A₃-heteroaryl, A₂-substituted aryl-A₃-heteroaryl, substituted aryl-A₃-heterocycloalkyl, A₂-substituted aryl-A₃-heterocycloalkyl,

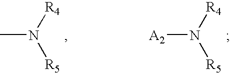

n and o are one or two provided that the sum of n plus o is two or three;

v and w are one, two, or three provided that the sum of v plus w is three, four, or five;

R₈ is hydrogen, halo, amino, —NH(lower alkyl), —N(lower alkyl)₂, nitro, alkyl, substituted alkyl, alkoxy, hydroxy, aryl, substituted aryl, A₂-aryl, A₂-substituted aryl, aryl-A₃-aryl, A₂-aryl-A₃-aryl, cycloalkyl, substituted cycloalkyl, A₂-cycloalkyl, A₂-substituted cycloalkyl, heteroaryl, A₂-heteroaryl, heterocycloalkyl, A₂-heterocycloalkyl, aryl-A₃-cycloalkyl, A₂-aryl-A₃-cycloalkyl, aryl-A₃-heteroaryl, A₂-aryl-A₃-heteroaryl, aryl-A₃-heterocycloalkyl, or A₂-aryl-A₃-heterocycloalkyl;

B₁, B₂ and B₃ are each CH, or two of B₁, B₂ and B₃ are CH and the other is N, or one of B₁, B₂ and B₃ is CH and the other two are N;

R₉ is hydrogen or lower alkyl;

R₁₀ is alkyl, substituted alkyl, alkyl-O-alkyl, alkyl-O-alkyl-O-alkyl, cycloalkyl, substituted cycloalkyl, A₂-cycloalkyl, A₂-substituted cycloalkyl, aryl, substituted aryl, A₂-aryl, A₂-substituted aryl, aryl-A₃-aryl, A₂-aryl-A₃-aryl, heteroaryl, A₂-heteroaryl, heterocycloalkyl, A₂-heterocycloalkyl, aryl-A₃-cycloalkyl, A₂-aryl-A₃-cycloalkyl, aryl-A₃-heteroaryl, A₂-aryl-A₃-heteroaryl, aryl-A₃-heterocycloalkyl or A₂-aryl-A₃-heterocyloalkyl;

R₂₀ is alkyl, substituted alkyl, cycloalkyl, substituted cycloalkyl, A₂-cycloalkyl, A₂-substituted cycloalkyl, A₂-aryl, or A₂-substituted aryl;

R₂₁ and R₂₂ are independently selected from hydrogen, alkyl, substituted alkyl, cycloalkyl, substituted cycloalkyl, A₂-cycloalkyl, A₂-substituted cycloalkyl, A₂-aryl, and A₂-substituted aryl;

p is an integer from 2 to 6;

q is an integer from 1 to 6;

r is zero, 1 or 2;

s is 1 or 2;

t is 1, 2, 3 or 4;

u is 1, 2 or 3;

A₂ is an alkylene or a substituted alkylene bridge of 1 to 10 carbons, an alkenyl or substituted alkenyl bridge of 2 to 10 carbons having one or more double bonds, or an alkynyl or substituted alkynyl bridge of 2 to 10 carbons having one or more triple bonds;

A₃ is a bond, an alkylene or a substituted alkylene bridge of 1 to 10 carbons, an alkenyl or substituted alkenyl bridge of 2 to 10 carbons having one or more double bonds, an alkynyl or substituted alkynyl bridge of 2 to 10 carbons having one or more triple bonds,

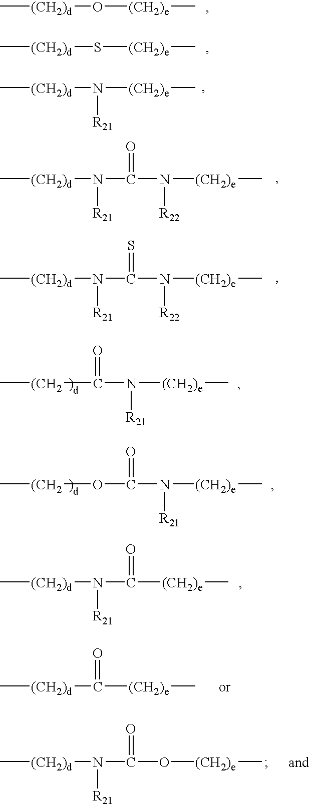

d and e are independently selected from zero and an integer from 1 to 10 provided that the sum of d plus e is no greater than 10.

### SUMMARY OF THE INVENTION

This invention is directed to the novel beta lactam compounds of formula I shown below and to a method for the use of such compounds as inhibitors of various in vivo enzyme systems including tryptase, thrombin, trypsin, Factor Xa, Factor VIIa, and urokinase-type plasminogen activator and their use in treating and/or preventing asthma and/or allergic rhinitis.

Compounds of this invention have the formula:

I.

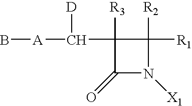

wherein

D is H or OR^{a};

R^{a} is H or alkyl;

A is a linear string of A¹, A², A³, A⁴, A⁵, A⁶, A⁷ and/or A⁸, in any order, such that A¹ may occur in the string from 0 to 6 times;

A² may occur in the string from 0 to 2 times;

A³, A⁴, A⁵, A⁶, A⁷ and/or A⁸ may each occur in the string 0 or 1 time, such that the total number of linear A groups is 0 to 6;

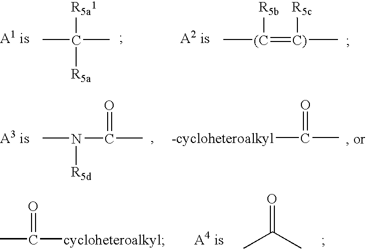

A⁵ is cycloalkyl;

A⁶ is aryl;

A⁷ is heteroaryl;

A⁸ is cycloheteroalkyl

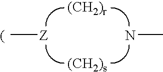

where Z is CH or N, r is 0 to 3 and s is 0 to 3) wherein R₅ₐ, R₅ₐ¹, R_{5b}, R_{5c}, and R_{5d} are the same or different and are H, alkyl, aryl, arylalkyl, halo or NO₂;

B is amino, aminoalkyl, amino cycloalkyl, cycloheteroalkyl, aryl, heteroaryl, alkylamino, carboxamido

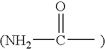

or cycloalkyl;

R₁ is hydrogen, carboxy, alkoxycarbonyl, A₂-aryl, alkyl,

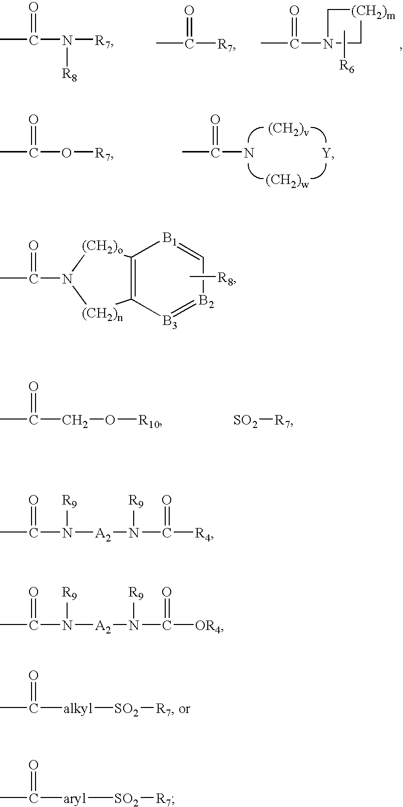

R₂ and R₃ are the same or different and are independently hydrogen or alkyl;

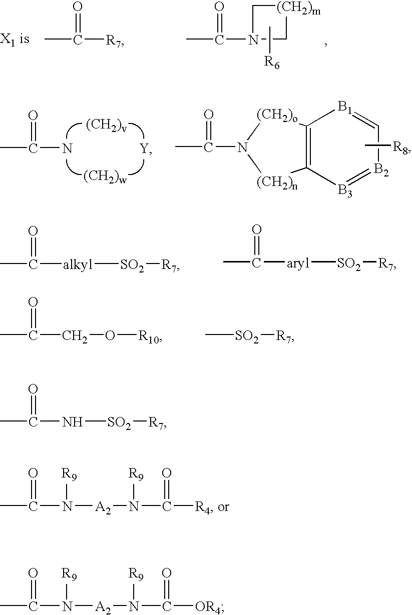

R₄ and R₅ are the same or different and are independently selected from hydrogen, alkyl, substituted alkyl, cycloalkyl, substituted cycloalkyl, A₂-cycloalkyl, A₂-substituted cycloalkyl, aryl, substituted aryl, A₂-aryl, A₂-substituted aryl, heteroaryl, A₂-heteroaryl, heterocycloalkyl, A₂-heterocycloalkyl, aryl-A₃-aryl, A₂-aryl-A₃-aryl, aryl-A₃-cycloalkyl, A₂-aryl-A₃-cycloalkyl, aryl-A₃-heteroaryl, A₂-aryl-A₃-heteroaryl, aryl-A₃-heterocycloalkyl, A₂-aryl-A₃-heterocycloalkyl, aryl-A₃-substituted aryl, A₂-aryl-A₃-substitued aryl, aryl-A₃-substituted cycloalkyl, A₂-aryl-A₃-substituted cycloalkyl, cycloalkyl-A₃-cycloalkyl, A₂-cycloalkyl-A₃-cycloalkyl, cycloalkyl-A₃-aryl, A₂-cycloalkyl-A₃-aryl, cycloalkyl-A₃-heteroaryl, A₂-cycloalkyl-A₃-heteroaryl, cycloalkyl-A₃-heterocycloalkyl, A₂-cycloalkyl-A₃-heterocycloalkyl, cycloalkyl-A₃-substituted cycloalkyl, A₂-cycloalkyl-A₃-substituted cycloalkyl, cycloalkyl-A₃-substituted aryl, A₂-cycloalkyl-A₃-substituted aryl, substituted cycloalkyl-A₃-cycloalkyl, A₂-substituted cycloalkyl-A₃-cycloalkyl, substituted cycloalkyl-A₃-substituted cycloalkyl, A₂-substituted cycloalkyl-A₃-substituted cycloalkyl, substituted cycloalkyl-A₃-aryl, A₂-substituted cycloalkyl-A₃-aryl, substituted cycloalkyl-A₃-heteroaryl, A₂-substituted cycloalkyl-A₃-heteroaryl, substituted cycloalkyl-A₃-heterocycloalkyl, A₂-substituted cycloalkyl-A₃-heterocycloalkyl, substituted cycloalkyl-A₃-substituted aryl, A₂-substituted cycloalkyl-A₃-substituted aryl, heteroaryl-A₃-heteroaryl, A₂-heteroaryl-A₃-heteroaryl, heteroaryl-A₃-cycloalkyl, A₂-heteroaryl-A₃-cycloalkyl, heteroaryl-A₃-substituted cycloalkyl, A₂-heteroaryl-A₃-substituted cycloalkyl, heteroaryl-A₃-aryl, A₂-heteroaryl-A₃-aryl, heteroaryl-A₃-heterocycloalkyl, A₂-heteroaryl-A₃-heterocycloalkyl, heteroaryl-A₃-substituted aryl, A₂-heteroaryl-A₃-substituted aryl, heterocycloalkyl-A₃-heterocycloalkyl, A₂-heterocycloalkyl-A₃-heterocycloalkyl, heterocycloalkyl-A₃-cycloalkyl, A₂-heterocycloalkyl-A₃-cycloalkyl, heterocycloalkyl-A₃-substituted cycloalkyl, A₂-heterocycloalkyl-A₃-substituted cycloalkyl, heterocycloalkyl-A₃-aryl, A₂-heterocycloalkyl-A₃-aryl, heterocycloalkyl-A₃-substituted aryl, A₂-heterocycloalkyl-A₃-substituted aryl, heterocycloalkyl-A₃-heteroaryl, A₂-heterocycloalkyl-A₃-heteroaryl, substituted aryl-A₃-substituted aryl, A₂-substituted aryl-A₃-substituted aryl, substituted aryl-A₃-cycloalkyl, A₂-substituted aryl-A₃-cycloalkyl, substituted aryl-A₃-substituted cycloalkyl, A₂-substituted aryl-A₃-substituted cycloalkyl, substituted aryl-A₃-aryl, A₂-substituted aryl-A₃-aryl, substituted aryl-A₃-heteroaryl, A₂-substituted aryl-A₃-heteroaryl, substituted aryl-A₃-heterocycloalkyl, and A₂-substituted aryl-A₃-heterocycloalkyl;

R₆ is hydrogen, alkyl, substituted alkyl, cycloalkyl, substituted cycloalkyl, A₂-cycloalkyl, A₂-substituted cycloalkyl, aryl, substituted aryl, A₂-aryl, A₂-substituted aryl, aryl-A₃-aryl, A₂-aryl-A₃-aryl, heteroaryl, A₂-heteroaryl, heterocycloalkyl, A₂-heterocycloalkyl, aryl-A₃-cycloalkyl, A₂-aryl-A₃-cycloalkyl, aryl-A₃-heteroaryl, A₂-aryl-A₃-heteroaryl, aryl-A₃-heterocycloalkyl, A₂-aryl-A₃-heterocycloalkyl, carboxy, alkoxycarbonyl, aryloxycarbonyl,

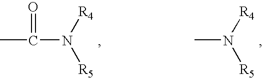

alkoxycarbonylamino, aryloxycarbonylamino, arylcarbonylamino, —N(alkyl)(alkoxycarbonyl), —N(alkyl)(aryloxycarbonyl), alkylcarbonylamino, —N(alkyl)(alkylcarbonyl), or —N(alkyl)(arylcarbonyl);

m is an integer from 1 to 5;

Y is O, S, N—R₄, N—SO₂—R₇,

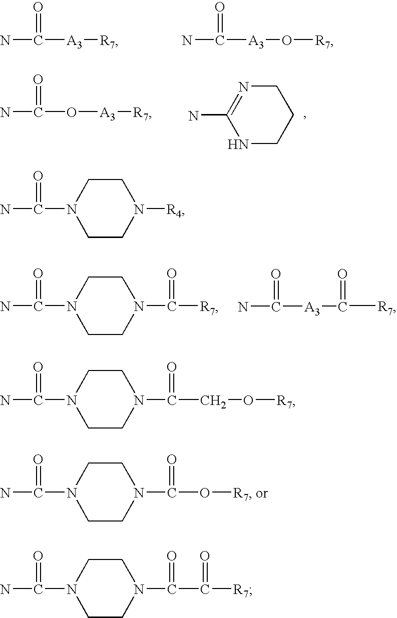

R₇ is H, alkyl, substituted alkyl, cycloalkyl, substituted cycloalkyl, A₂-cycloalkyl, A₂-substituted cycloalkyl, aryl, substituted aryl, A₂-aryl, A₂-substituted aryl, heteroaryl, A₂-heteroaryl, heterocycloalkyl, A₂-heterocycloalkyl, aryl-A₃-aryl, A₂-aryl-A₃-aryl, aryl-A₃-cycloalkyl, A₂-aryl-A₃-cycloalkyl, aryl-A₃-heteroaryl, A₂-aryl-A₃-heteroaryl, aryl-A₃-heterocycloalkyl, A₂-aryl-A₃-heterocycloalkyl, aryl-A₃-substituted aryl, A₂-aryl-A₃-substitued aryl, aryl-A₃-substituted cycloalkyl, A₂-aryl-A₃-substituted cycloalkyl, cycloalkyl-A₃-cycloalkyl, A₂-cycloalkyl-A₃-cycloalkyl, cycloalkyl-A₃-aryl, A₂-cycloalkyl-A₃-aryl, cycloalkyl-A₃-heteroaryl, A₂-cycloalkyl-A₃-heteroaryl, cycloalkyl-A₃-heterocycloalkyl, A₂-cycloalkyl-A₃-heterocycloalkyl, cycloalkyl-A₃-substituted cycloalkyl, A₂-cycloalkyl-A₃-substituted cycloalkyl, cycloalkyl-A₃-substituted aryl, A₂-cycloalkyl-A₃-substituted aryl, substituted cycloalkyl-A₃-cycloalkyl, A₂-substituted cycloalkyl-A₃-cycloalkyl, substituted cycloalkyl-A₃-substituted cycloalkyl, A₂-substituted cycloalkyl-A₃-substituted cycloalkyl, substituted cycloalkyl-A₃-aryl, A₂-substituted cycloalkyl-A₃-aryl, substituted cycloalkyl-A₃-heteroaryl, A₂-substituted cycloalkyl-A₃-heteroaryl, substituted cycloalkyl-A₃-heterocycloalkyl, A₂-substituted cycloalkyl-A₃-heterocycloalkyl, substituted cycloalkyl-A₃-substituted aryl, A₂-substituted cycloalkyl-A₃-substituted aryl, heteroaryl-A₃-heteroaryl, A₂-heteroaryl-A₃-heteroaryl, heteroaryl-A₃-cycloalkyl, A₂-heteroaryl-A₃-cycloalkyl, heteroaryl-A₃-substituted cycloalkyl, A₂-heteroaryl-A₃-substituted cycloalkyl, heteroaryl-A₃-aryl, A₂-heteroaryl-A₃-aryl, heteroaryl-A₃-heterocycloalkyl, A₂-heteroaryl-A₃-heterocycloalkyl, heteroaryl-A₃-substituted aryl, A₂-heteroaryl-A₃-substituted aryl, heterocycloalkyl-A₃-heterocycloalkyl, A₂-heterocycloalkyl-A₃-heterocycloalkyl, heterocycloalkyl-A₃-cycloalkyl, A₂-heterocycloalkyl-A₃-cycloalkyl, heterocycloalkyl-A₃-substituted cycloalkyl, A₂-heterocycloalkyl-A₃-substituted cycloalkyl, heterocycloalkyl-A₃-aryl, A₂-heterocycloalkyl-A₃-aryl, heterocycloalkyl-A₃-substituted aryl, A₂-heterocycloalkyl-A₃-substituted aryl, heterocycloalkyl-A₃-heteroaryl, A₂-heterocycloalkyl-A₃-heteroaryl, substituted aryl-A₃-substituted aryl, A₂-substituted aryl-A₃-substituted aryl, substituted aryl-A₃-cycloalkyl, A₂-substituted aryl-A₃-cycloalkyl, substituted aryl-A₃-substituted cycloalkyl, A₂-substituted aryl-A₃-substituted cycloalkyl, substituted aryl-A₃-aryl, A₂-substituted aryl-A₃-aryl, substituted aryl-A₃-heteroaryl, A₂-substituted aryl-A₃-heteroaryl, substituted aryl-A₃-heterocycloalkyl, A₂-substituted aryl-A₃-heterocycloalkyl,

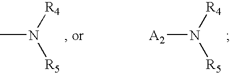

n and o are one or two provided that the sum of n plus o is two or three;

v and w are one, two, or three provided that the sum of v plus w is three, four, or five;

R₈ is hydrogen, halo, amino, —NH(lower alkyl), —N(lower alkyl)₂, nitro, alkyl, substituted alkyl, alkoxy, hydroxy, aryl, substituted aryl, A₂-aryl, A₂-substituted aryl, aryl-A₃-aryl, A₂-aryl-A₃-aryl, cycloalkyl, substituted cycloalkyl, A₂-cycloalkyl, A₂-substituted cycloalkyl, heteroaryl, A₂-heteroaryl, heterocycloalkyl, A₂-heterocycloalkyl, aryl-A₃-cycloalkyl, A₂-aryl-A₃-cycloalkyl, aryl-A₃-heteroaryl, A₂-aryl-A₃-heteroaryl, aryl-A₃-heterocycloalkyl, or A₂-aryl-A₃-heterocycloalkyl;

B₁, B₂ and B₃ are each CH, or two of B₁, B₂ and B₃ are CH and the other is N, or one of B₁, B₂ and B₃ is CH and the other two are N;

R₉ is hydrogen or lower alkyl;

R₁₀ is alkyl, substituted alkyl, alkyl-O-alkyl, alkyl-O-alkyl-O-alkyl, cycloalkyl, substituted cycloalkyl, A₂-cycloalkyl, A₂-substituted cycloalkyl, aryl, substituted aryl, A₂-aryl, A₂-substituted aryl, aryl-A₃-aryl, A₂-aryl-A₃-aryl, heteroaryl, A₂-heteroaryl, heterocycloalkyl, A₂-heterocycloalkyl, aryl-A₃-cycloalkyl, A₂-aryl-A₃-cycloalkyl, aryl-A₃-heteroaryl, A₂-aryl-A₃-heteroaryl, aryl-A₃-heterocycloalkyl or A₂-aryl-A₃-heterocycloalkyl;

R₂₁ and R₂₂ are the same or different and are independently selected from hydrogen, alkyl, substituted alkyl, cycloalkyl, substituted cycloalkyl, A₂-cycloalkyl, A₂-substituted cycloalkyl, A₂-aryl, and A₂-substituted aryl;

p is an integer from 2 to 6;

q is an integer from 1 to 6;

r is zero, 1, 2 or 3;

s is 1, 2 or 3;

t is 1, 2, 3 or 4;

u is 1, 2 or 3;

A₂ is an alkylene or a substituted alkylene bridge of 1 to 10 carbons, an alkenyl or substituted alkenyl bridge of 2 to 10 carbons having one or more double bonds, or an alkynyl or substituted alkynyl bridge of 2 to 10 carbons having one or more triple bonds;

A₃ is a bond, an alkylene or a substituted alkylene bridge of 1 to 10 carbons, an alkenyl or substituted alkenyl bridge of 2 to 10 carbons having one or more double bonds, an alkynyl or substituted alkynyl bridge of 2 to 10 carbons having one or more triple bonds;

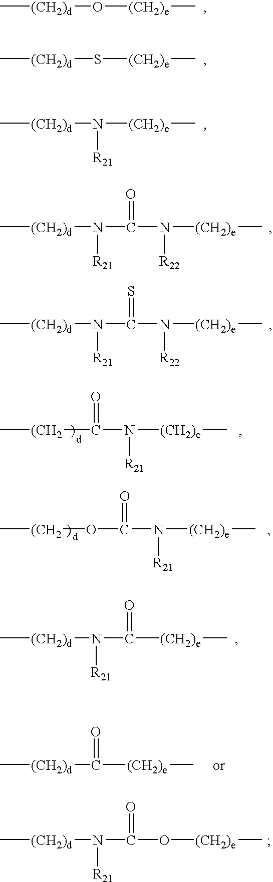

d and e are independently selected from zero and an integer from 1 to 10 provided that the sum of d plus e is no greater than 10;

and an inner salt or a pharmaceutically acceptable salt thereof, a hydrolyzable ester thereof, or a solvate thereof, with the provisos that

I. where R₁ is COOZ, where Z is

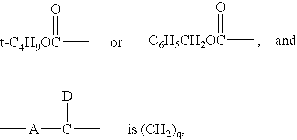

then B is other than amino or R₂₀—NH— where R₂₀ is alkyl, cycloalkyl, A₂-cycloalkyl or A₂-aryl;

II. where R₁ is

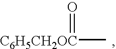

X₁ is

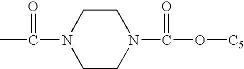

C₉ alkyl, and

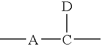

is other than

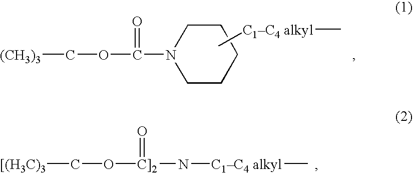

(3) amino C₁-C₅ alkyl,

(4) C₁-C₄ alkylamino C₁-C₅ alkyl, or

(5) piperidyl.

Preferred are compounds of formula I wherein

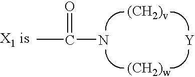

where Y is

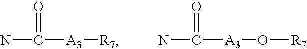

(where A₃ is a bond) and R₇ is alkyl (preferably (di-isopropyl) methyl)), cycloalkyl, aryl or arylalkyl, or Y is

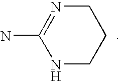

More preferred are compounds of formula I where X₁ is as defined above and where:

R₁ is carboxy, benzyloxycarbonyl,

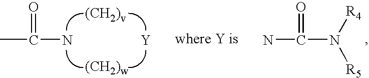

R₂ is hydrogen,

R₃ is hydrogen,

D is hydrogen,

A is

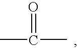

a bond, alkylene, -cycloheteroalkyl

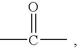

or heteroaryl, such as pyridyl, and

B is amino, cycloheteroalkyl, preferably

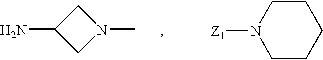

(where Z₁ is H, or NH₂CO— or alkyl), or

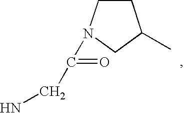

or heteroaryl, preferably pyridyl.

Most preferred are compounds of formula I where X₁ includes the moiety

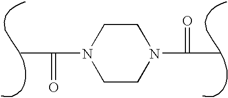

In the compounds of formula I of the invention and intermediates disclosed herein, the A₁, A₂, and A₃ groups as employed to define various substituents in the same compound or different compound may be the same or different and are independently selected from the various groups covered by the A₁, A₂, and A₃ groups.

Examples of preferred compounds include the following:

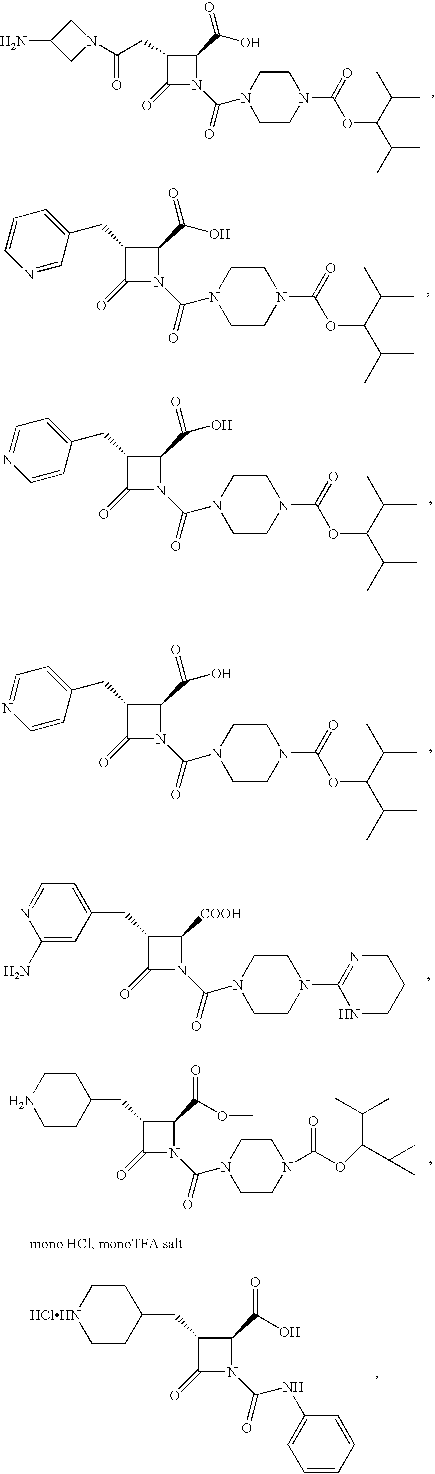

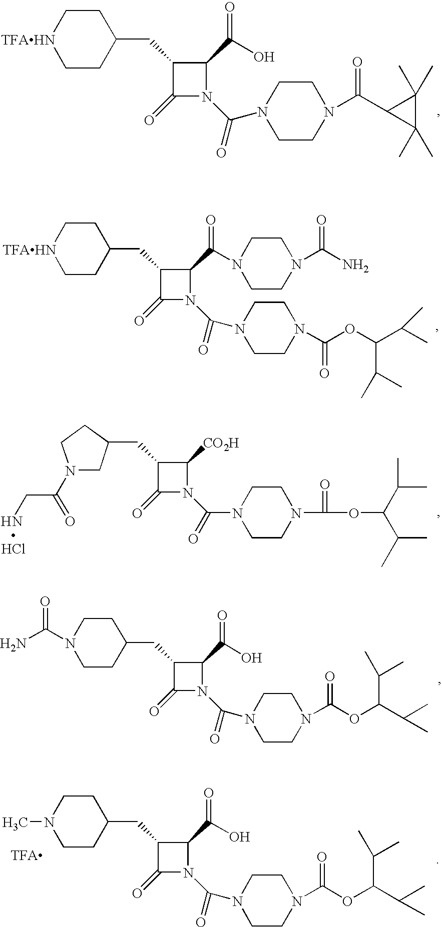

Examples of preferred compounds having tryptase inhibition activity include, but are not limited to,

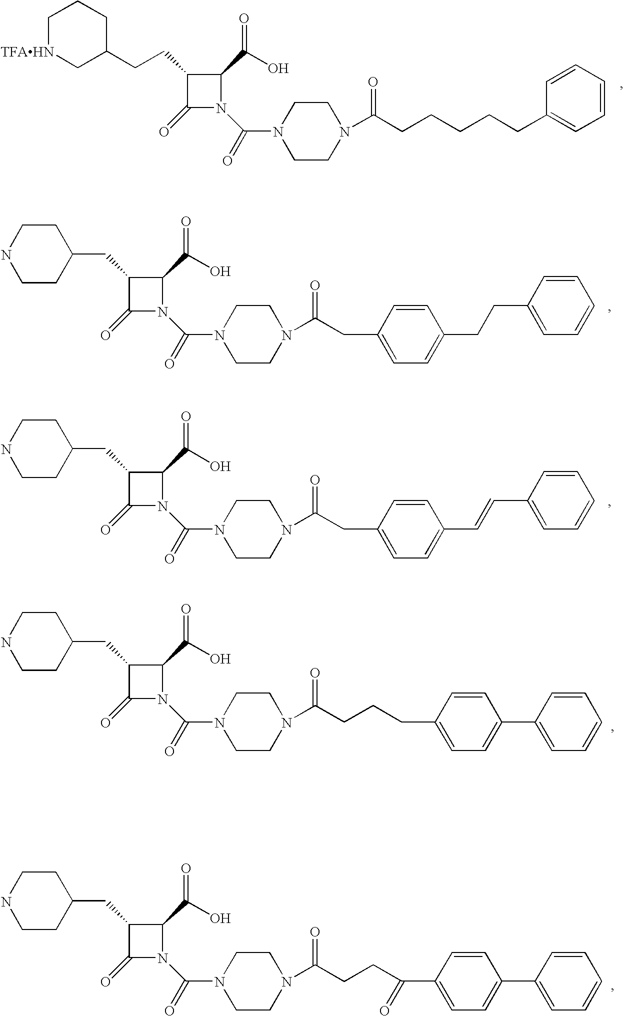

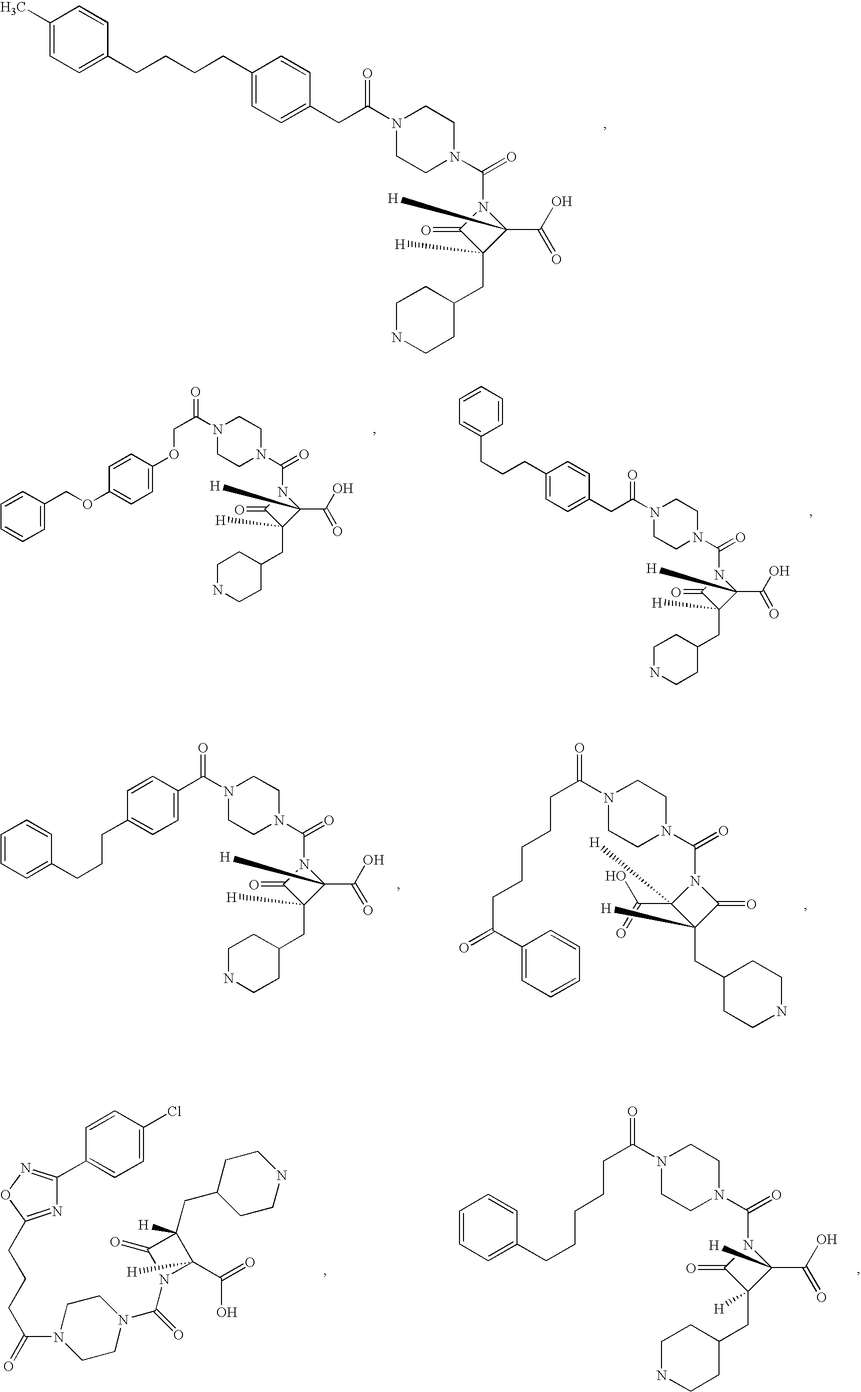

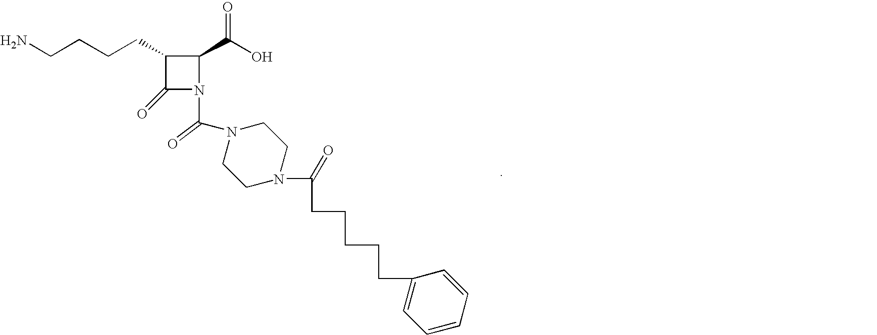

In addition, in accordance with the present invention, a method is provided for treating and/or preventing medical conditions in a mammalian species such as humans, dogs and cats, related to tryptase, thrombin, trypsin, Factor Xa, Factor VIIa, or urokinase-type plasminogen activator and/or treating and/or preventing asthma, including acute asthma and chronic asthma, or allergic rhinitis, wherein a therapeutically effective amount of a compound of formula I (which may or may not be limited by provisos I. and II. set out above) is administered to mammalian species in need of treatment.

### DETAILED DESCRIPTION OF THE INVENTION

The term “alkyl” includes “substituted alkyl” and refers to straight or branched chain radicals having up to ten carbon atoms which may include one or more substitutents as described below. The term “lower alkyl” refers to straight or branched radicals having up to four carbon atoms and is a preferred subgrouping for the term alkyl.

The term “substituted alkyl” refers to such straight or branched chain radicals of 1 to 10 carbons wherein one or more, preferably one, two or three, hydrogens have been replaced by a hydroxy, amino, cyano, halo, aryl, trifluoromethyl, nitro, —NH(lower alkyl), —N(lower alkyl)₂, alkoxy, alkylthio, carboxy, alkoxycarbonyl, aminocarbonyl, alkoxycarbonylamino, cycloalkyl, carboxamido, formyl or acyl.

The term “alkoxy” refers to such alkyl groups as defined above attached to an oxygen. The term “alkylthio” refers to such alkyl groups as defined above attached to a sulfur. The terms “lower alkoxy” and “lower alkylthio” refer to such lower alkyl groups as defined above attached to an oxygen or sulfur.

The term “cycloalkyl” includes “substituted cycloalkyl” and refers to fully or partially saturated rings of 3 to 7 carbons, which may include one or more substitutents as described below.

The term “substituted cycloalkyl” refers to such rings of 3 to 7 carbons having one or more substituents selected from lower alkyl, lower alkoxy, lower alkylthio, halo, hydroxy, trifluoromethyl, nitro, cyano, amino, aryl, cycloalkyl, carboxamido, formyl, acyl, —NH(lower alkyl), —N(lower alkyl)₂, or carboxy as well as such rings fused to a phenyl ring such as tetrahydronaphthyl.

The term “aryl” includes substituted aryl and refers to phenyl, 1-naphthyl and 2-naphthyl, which may include one or more substitutents as described below.

The term “substituted aryl” refers to phenyl, 1-naphthyl, and 2-naphthyl having a substituent selected from alkyl of 1 to 10 carbons, lower alkyl, lower alkoxy, lower alkylthio, halo, hydroxy, trifluoromethyl, nitro, amino, aminoalkyl, cycloalkyl, carboxamido, formyl, acyl, —NH(loweralkyl), —N(lower alkyl)₂, or carboxy, aryl, and di and tri-substituted phenyl, 1-naphthyl, or 2-naphthyl wherein said substituents are selected from methyl, methoxy, methylthio, halo, hydroxy and amino.

The term “heteroaryl” refers to unsaturated and partially saturated rings of 4 to 7 atoms containing one or two O and S atoms and/or one to four N atoms, one to three N atoms when the ring is 4 atoms, provided that the total number of hetero atoms in the ring is 4 or less, 3 or less when the ring is 4 atoms. The heteroaryl ring is attached by way of an available carbon or nitrogen atom. Preferred heteroaryl groups include 2-, 3-, or 4-pyridyl, 4-imidazolyl, 4-thiazolyl, 2- and 3-thienyl, 2- and 3-furyl, and 2-(1,4,5,6-tetrahydropyrimidinyl). The term heteroaryl also includes bicyclic rings wherein the 4 to 7 membered ring containing O, S and N atoms as defined above is fused to a benzene, cycloalkyl, heteroaryl or heterocycloalkyl ring. Preferred bicyclic rings are 2- and 3-indolyl and 4- and 5-quinolinyl. The mono or bicyclic heteroaryl ring can also be additionally substituted at one, two, three or more available carbon atoms by a lower alkyl, aryl, halo, carboxy, amino, hydroxy, A₂-lower alkoxy, A₂-guanido, benzyl, keto, cycloalkyl, carboxamido, formyl, acyl, or cyclohexylmethyl. Also, if the mono or bicyclic ring has an available N-atom such N atom can also be substituted by an N-protecting group such as benzyloxycarbonyl, tert-butoxycarbonyl, benzyl or benzhydryl.

The term “heterocycloalkyl” or “cycloheteroalkyl” refers to fully saturated rings of 4 to 7 atoms containing one or two O and S atoms and/or one to four N atoms, one to three N atoms when the ring is 4 atoms, provided that the total number of hetero atoms in the ring is 4 or less, 3 or less when the ring is 4 atoms. The heterocycloalkyl is attached by way of an available carbon or nitrogen atom. Preferred heterocycloalkyl groups include pyrrolidinyl, tetrahydrofuranyl, tetrahydrothienyl, morpholinyl, tetrahydro-1,2-thiazinyl, piperazinyl, piperidinyl, homopiperizinyl and azetidinyl. The term heterocycloalkyl also includes bicyclic rings wherein the 4 to 7 membered saturated ring containing O, S and N atoms as defined above is fused to a cycloalkyl, benzene, heteroaryl, or heterocycloalkyl ring. The mono or bicyclic heterocycloalkyl ring can also be substituted at one or more available carbon atoms by a lower alkyl, halo, carboxy, hydroxy, keto, amino, aryl, cycloalkyl, carboxamido, formyl, acyl, aminocarbonyl, aminoalkylcarbonyl, A₂-lower alkoxy, A₂-guanido, benzyl or cyclohexylmethyl. Also, if the mono or bicyclic heterocycloalky ring has an available N atom such N atom can also be substituted by an N-protecting group such as benzyloxycarbonyl, tert-butoxycarbonyl, benzyl or benzhydryl.

The term “halo” refers to chloro, bromo, fluoro and iodo.

The terms “alkylene” and “substituted alkylene” refer to a bridge of 1 to 10 carbons such as —CH₂—, —(CH₂)₂—, —(CH₂)₉—, etc. One or more hydrogens, preferably one, in the alkylene bridge can be replaced by an alkyl, substituted alkyl, carboxy, alkoxycarbonyl, amino, —NH(lower alkyl), —N(lower alkyl)₂, hydroxy, aminocarbonyl, alkoxycarbonylamino, halo, cycloalkyl, substituted cycloalkyl, aryl, substituted aryl, hetereoaryl, or heterocycloalkyl, e.g.

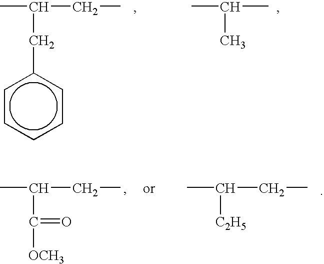

The term “alkenyl” includes “substituted alkenyl” and refers to a bridge of 2 to 10 carbons having one or more double bonds, preferably 2 to 6 carbons with one double bond, such as —CH═CH—, —CH═CH—CH₂—, —CH₂—CH═CH—, etc. One or more hydrogens, preferably one, in the alkenyl bridge can be replaced by an alkyl, substituted alkyl, carboxy, alkoxycarbonyl, amino, —NH(lower alkyl), —N(lower alkyl)₂, hydroxy, aminocarbonyl, alkoxycarbonylamino, halo, cycloalkyl, substituted cycloalkyl, aryl, substituted aryl, heteroaryl, or heterocycloalkyl, e.g.

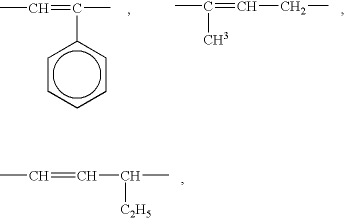

and the like.

The term “alkynyl” and “substituted alkynyl” refer to a bridge of 2 to 10 carbons having one or more triple bonds, preferably 2 to 6 carbons with one triple bond, such as —C═C—, —CH₂—C═C—, —C═C—CH₂—, etc. One or more hydrogens in the alkynyl bridge can be replaced by an alkyl, substituted alkyl, carboxy, alkoxycarbonyl, amino, carboxy, alkoxycarbonyl, amino, —NH(lower alkyl), —N(lower alkyl)₂, hydroxy, aminocarbonyl, alkoxycarbonylamino, halo, cycloalkyl, substituted cycloalkyl, aryl, substituted aryl, heteroaryl, or heterocycloalkyl, e.g.

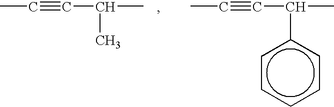

and the like.

The term “acyl” refers to alkyl, aryl, alkenyl, heteroaryl, cycloheteroalkyl or cycloalkyl—attached to a carbonyl group.

Compounds of formula I may be prepared by the methods in the following Schemes.

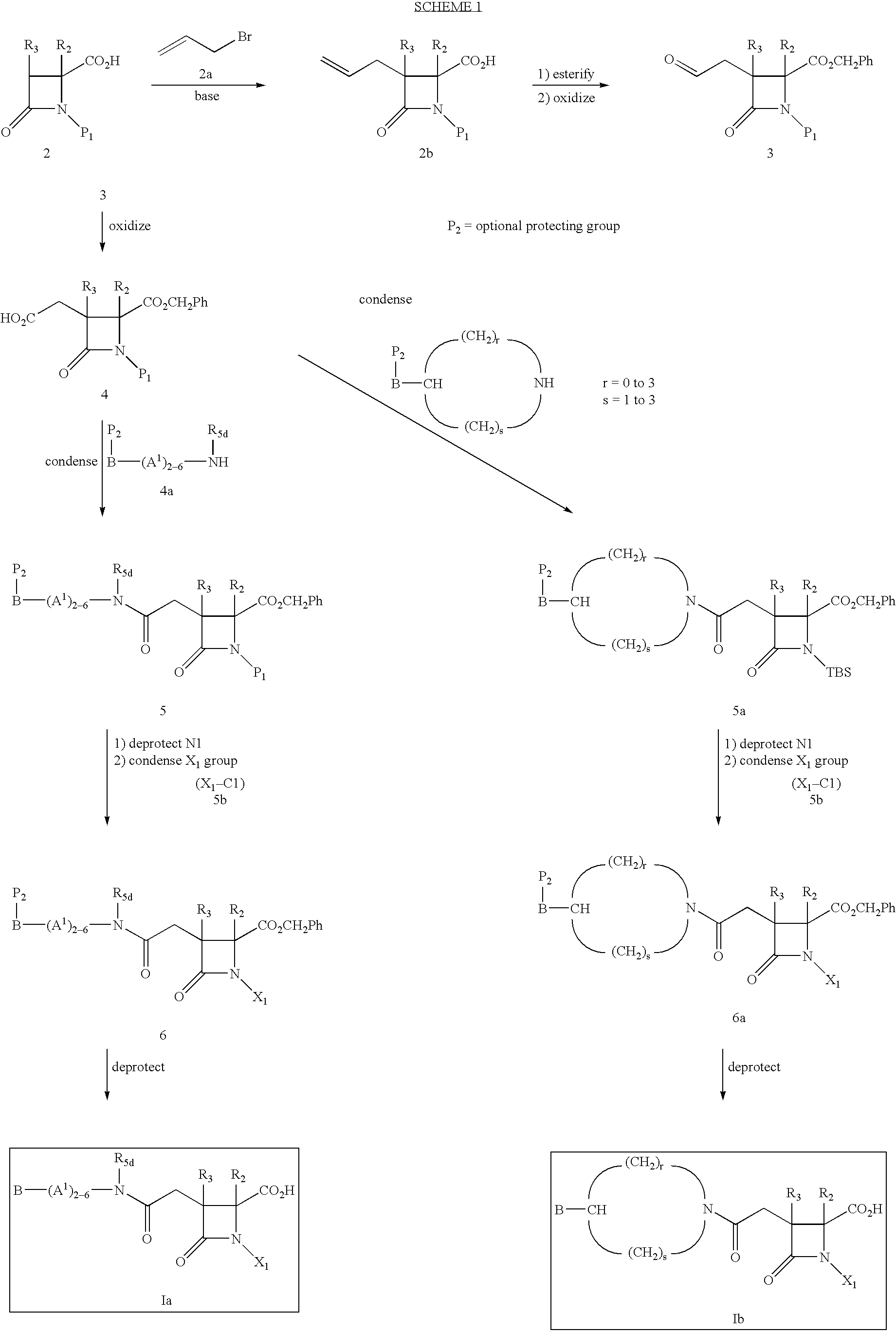

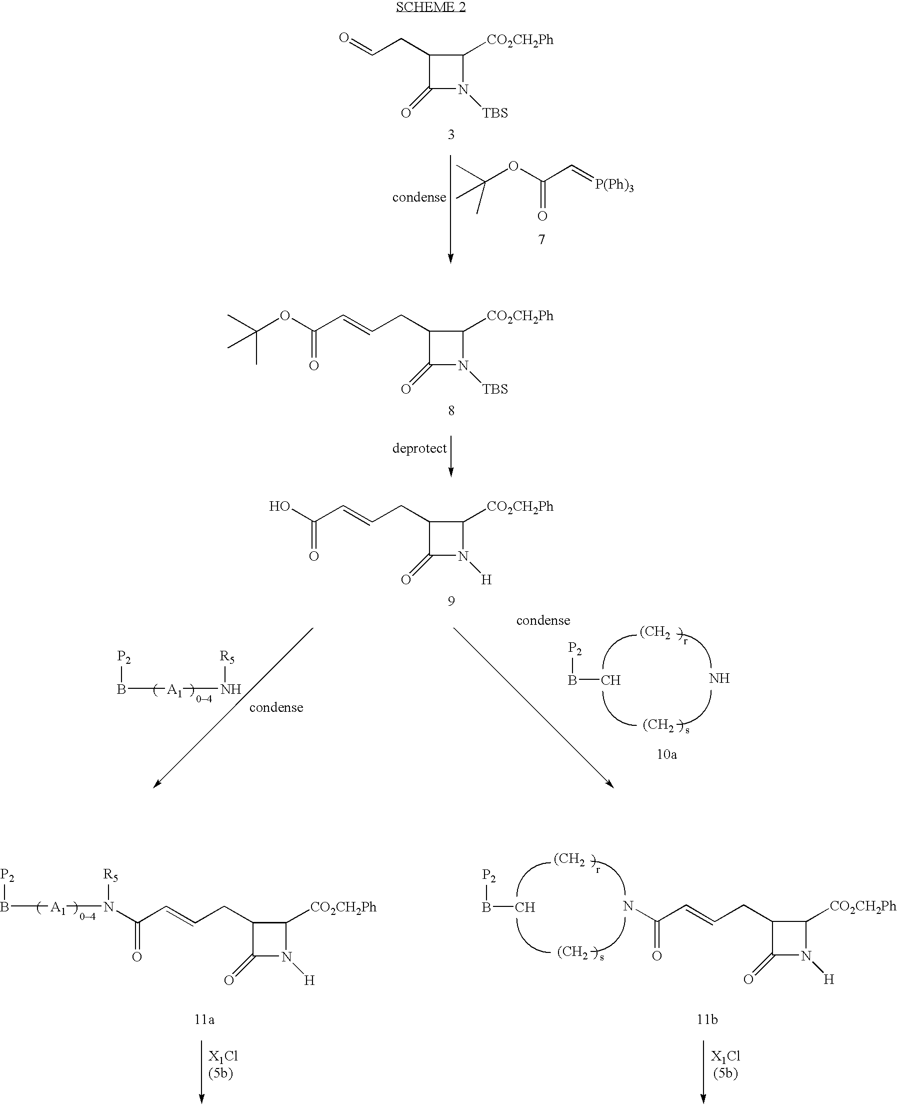

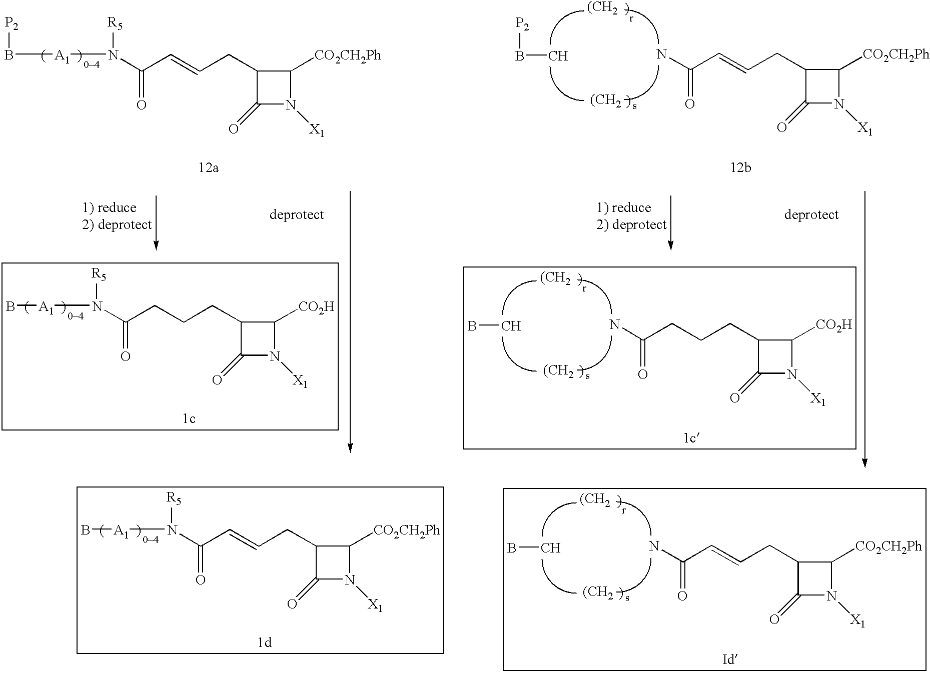

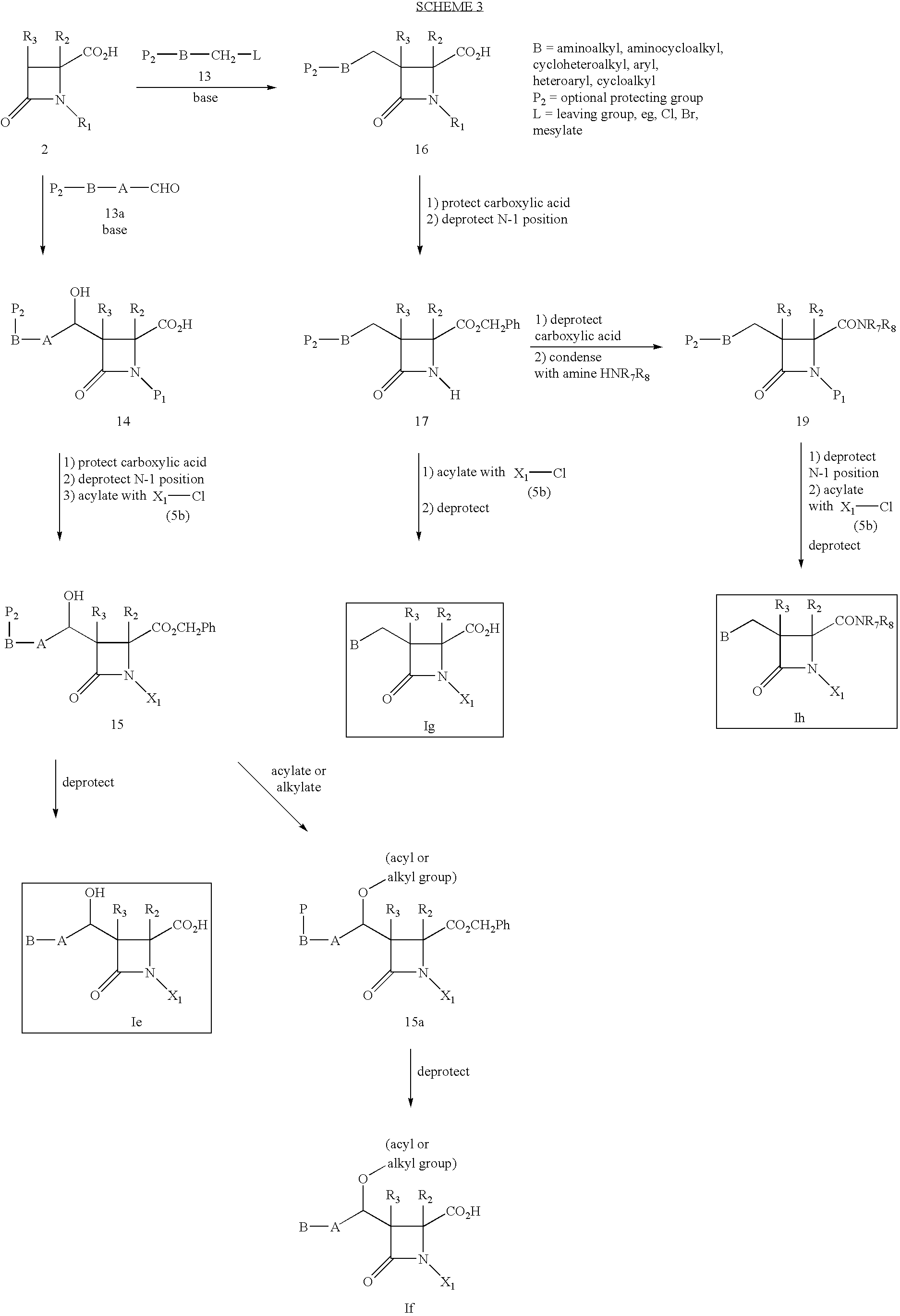

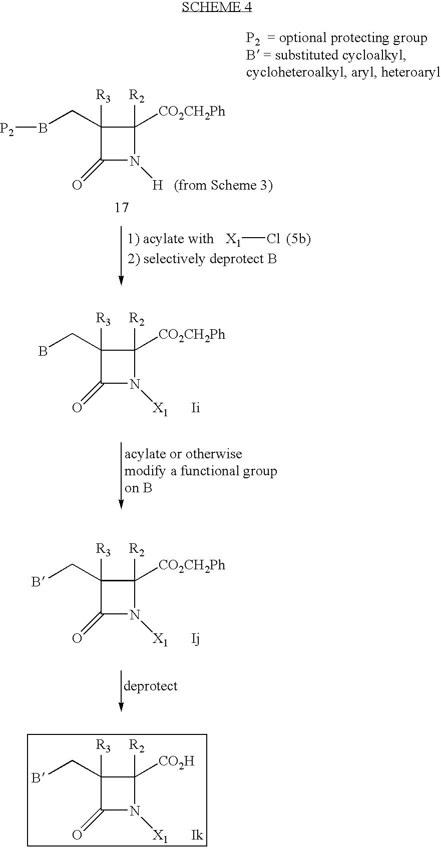

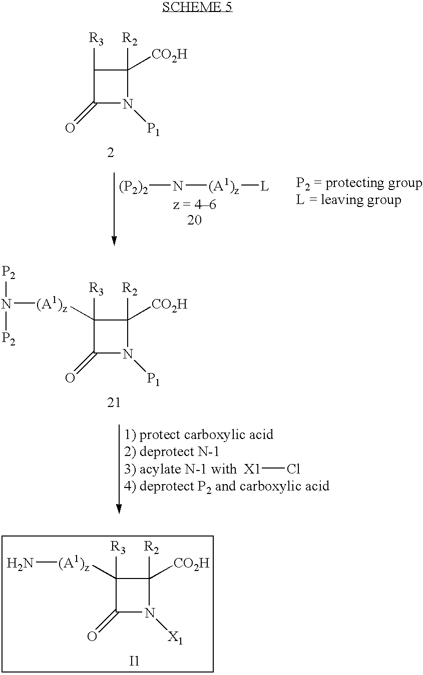

Compounds of the invention of formula I wherein A includes an aminocarbonyl function (that is A³) are prepared as shown in Scheme 1 and as described below.

The carboxy substituted azetidinone of the formula 2

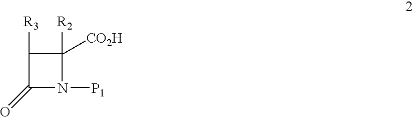

wherein P₁ is a silyl protecting group such as tert-butyldimethylsilyl (TBS) is treated with a propen-1-yl halide of the formula 2a

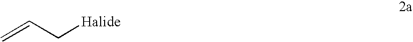

(preferably 3-bromo-1-propene) in the presence of base to give the carboxy substituted azetidinone of the formula 2b

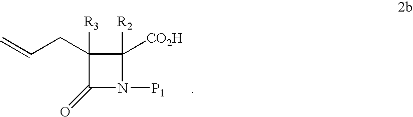

The carboxy substituted azetidinone of formula 2b is then treated esterified by treating 2b with benzyl alcohol and DCC or benzyl bromide and sodium bicarbonate to form ester 2c

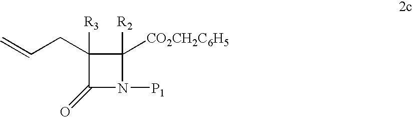

which is then oxidized by treating 2c with ozone and triphenylphospine to form azetidinone 3.

The azetidinone 3 is oxidized by treatment with potassium permanganate to form azetidinone 4. Coupling the azetidinone 4 with an amine selected from

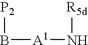

(where P₂ is an optional protecting group such as BOC or Cbz or coupling 4 with 4a such as

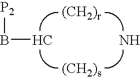

gives the compound of the formula 5 or 5a

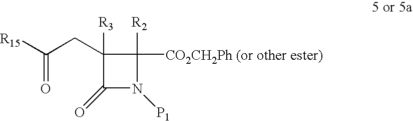

wherein R₁₅ is

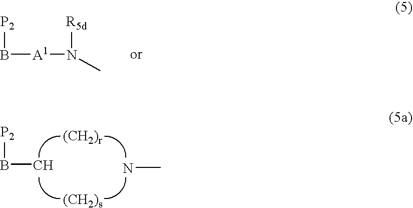

Reacting the intermediate of formula 5 or 5a with ammonium fluoride removes the P₁ protecting group and condensing the deprotected intermediate with X₁—Cl (5b) compound selected from

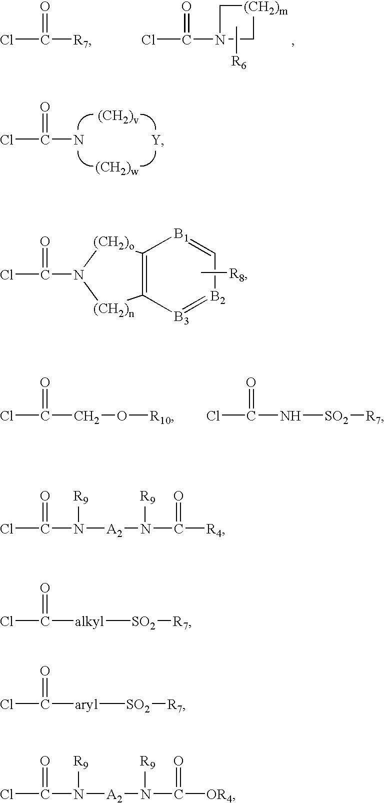

or Cl—SO₂—R₇ or reacting with OCN—SO₂—R₉ gives the compound of the formula 6 or 6a.

Removal of the B protecting group P₂ and the carboxylic acid protecting group gives the compounds of formula Ix

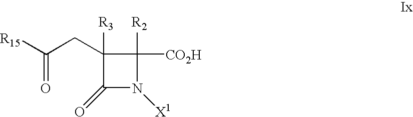

where R₁₅ is

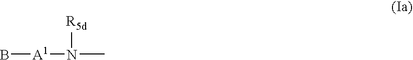

or where R₁₅ is

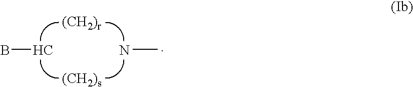

Compounds of formula I of the invention wherein A is -A³-A¹- and B is amino, or A is -A²-A¹- and B is amino may be prepared as shown in Scheme 2 by condensing 3 with an ester of the structure 7

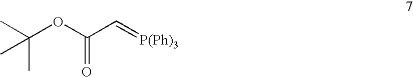

gives the azetidinone acid ester 8 which is deprotected by treating with ammonium fluoride followed by TFA to form azetidinone acid 9.

Coupling 9 with amine 10 or with 10a

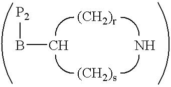

gives protected intermediates 11a and 11b, respectively.

Reacting the intermediate 11a and 11b with X₁—Cl compound of the formula 12a and 12b, respectively.

Compounds 12a and 12b are reduced by reaction with hydrogen and palladium as carbon and then deprotected by reaction with TFA to give compounds of the invention Ic and Ic′ respectively.

Where compounds 12a and 12 b are deprotected by reaction with TFA, compound of the invention Id and Id′ are produced respectively.

Compounds of formula I of the invention where D is OR^{a} may be prepared as shown in Scheme 3.

Azetidinone 2 is treated with aldehyde compound 13a in the presence of a base such as NaHMDS or LDA to form the intermediate 14. The carboxylic acid portion of 14 is protected by treating 14 with benzyl alcohol and DDC or benzyl bromide and NaHCO₃ to form an ester. The P₁ nitrogen protecting group is removed from the ester by treating the ester with ammonium fluoride. The resulting intermediate is treated with an X₁CI compound 5b (as employed to react with intermediate 11 in Scheme 2) to form compound 15 which is deprotected by reaction with hydrogen with palladium or carbon and treatment with TFA or HCl in dioxane to form compound Ie of the invention.

Alternatively compound 15 may first be acylated or alkylated to form compound 15a which is deprotected by treating with hydrogen with palladium or carbon and treatment with TFA or HCl in dioxane to form compound If of the invention.

Compounds of formula I of the invention where A is a bond may be prepared as shown in Scheme 3 as follows: Azetidinone 2 is treated with reactant 13 in the presence of a base such as NaHMDS or LDA to form intermediate 16. Protection of the carboxylic acid of 16 by reacting 16 with benzyl alcohol and DCC or benzyl bromide and NaHCO₃ and deprotection of the N-1 position by reacting acid protected 16 with ammonium fluoride or tetrabutyl ammonium fluoride produces intermediate 17. Intermediate 17 is acylated with an X₁Cl compound 5b (as employed to react with intermediate 11 in Scheme 2) and the P₂ protecting group and carboxylic acid protecting group are removed by reaction by either catalytic hydrogenation or sequential catalytic hydrogenation and treatment with TFA to form the compound of the invention Ig.

Compounds of the invention wherein A is a bond and R₁ is an aminocarbonyl may be prepared as shown in Scheme 3 as follows. The carboxylic acid protecting group in intermediate 17 is removed by catalytic hydrogenation and the deprotected compound is condensed with amine HNR₇R₈ and subsequently acylated by reaction with X₁—Cl (5b) to give the azetidinone 19. Removal of the P₂ protecting group by reacting with TFA or HCl in dioxane gives the compound of the invention Ih.

Compounds of the invention wherein A is a bond and B is as defined hereinbefore or B has a modified functional group (such as formed by acylation or reductive amination) is prepared as described in Scheme 4 as follows.

Azetidinone 17 is acylated with X₁—Cl (5b) as described hereinbefore and B is selectively deprotected by reacting with TFA or HCl in dioxane to form compound of the invention Ii. Compound Ii may then be acylated or a functional group or B may be modified (such as by acylation with an activated carboxylic acid or an isocyanate or by reductive amination with an aldehyde and palladium or carbon with hydrogen.) Acylation forms a compound of the invention Ij which is deprotected by reacting with catalytic hydrogenation to form compound of the invention Ik. Reductive amination in the presence of palladium on carbon and hydrogen results in concomitant deprotection of the carboxylic acid protecting group to give compound Ik.

Compounds of the invention of formula I wherein A is (A)_{z} where z is 4 to 6 and B is amino may be prepared as shown in Scheme 5 as follows. Azetidinone 2 is treated with a protected amine alkylating agent 20 to form protected amine 21. The carboxylic acid of 21 is protected by reacting 21 with benzyl bromide and sodium bicarbonate and N-1 is deprotected by reacting with ammonium fluoride or tetrabutyl ammonium fluoride and N-1 is acylated by reacting with X₁—Cl (5b). The amine and carboxylic acid protecting groups are removed together by catalytic hydrogenation or sequential treatment with TFA and catalytic hydrogenation to form amine compound of the invention Il.

The azetidinone compounds of formula I to Il and various intermediates and starting materials employed in their synthesis contain one or two asymmetric carbons as denoted below at ring positions 3 and 4

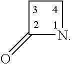

Of course, the compounds of formula I where R₁ and R₂ are the same and the compounds of formula I where R₁ is hydrogen contain only one asymmetric ring carbon. Additional asymmetric carbons may be present in the compounds of formula I to Il depending upon the definitions of the substituents R₁, A, X₁, R₂ and R₃. As is well known in the art, see for example J. March. Advanced Organic Chemistry, Fourth Edition, John Wiley & Sons, New York, N.Y. (1991), pages 94-164, such asymmetric carbon atoms give rise to enantiomers and diastereomers, and all such stereoisomers, either in pure form or in the form of mixtures, are included within the scope of this invention. In addition, when alkenes are present in the compounds of formula I to Il, they may, when appropriately substituted exist as cis or trans isomers, or as mixtures thereof. Again, all such forms are within the scope of this invention.

The compounds of formula I to Il can be obtained as a pharmaceutically acceptable salt, as a physiologically hydrolyzable ester, or as a solvate. The compounds of formulas I to Il wherein R₁ is carboxy can exist in the form of an inner salt or zwitterion. All such forms are within the scope of this invention. Pharmaceutically acceptable salts include salts with mineral acids such as hydrochloric, hydrobromic, phosphoric and sulfuric as well as salts with organic carboxylic acids or sulfonic acids such as acetic, trifluoroacetic, citric, maleic, oxalic, succinic, benzoic, tartaric, fumaric, mandelic, ascorbic, malic, methanesulfonic, p-toluensulfonic and the like. Preparation of these acid addition salts is carried out by conventional techniques.

The novel compounds of formulas I to Il of the invention possess tryptase inhibition activity. This activity was confirmed using either isolated human skin tryptase or recombinant human tryptase; prepared from the human recombinant beta-protryptase expressed by baculovirus in insect cells. The expressed beta-protryptase was purified using sequential immobilized heparin affinity resin followed by an immunoaffinity column using an anti-tryptase monoclonoal antibody. The protryptase was activated by auto-catalytic removal of the N-terminal in the presence of dextran sulfate followed by dipeptidyl peptidase I (DPPI) removal of the two N-terminal amino acids to give the mature active enzyme (Sakai et al., J. Clin. Invest., 97, pages 988-995, 1996). Essentially equivalent results were obtained using isolated native enzyme or the activated expressed enzyme. The tryptase enzyme was maintained in 2M sodium chloride, 10 nM 4-morpholinepropanesulfonic acid, pH 6.8.

The assay procedure employed a 96 well microplate. To each well of the microplate (Nunc MaxiSorp), 250 μl of assay buffer [containing low molecular weight heparin and tris (hydroxymethyl)aminomethane] was added followed by 2.0 μl of the test compound in dimethylsulfoxide. The substrate (10 μl) was then added to each well to give a final concentration of either 370 μM benzoyl-arginine-p-nitroaniline (BAPNA) or 100 μM benzyloxycarbonyl-glycine-proline-arginine-p-nitroaniline (CBz-Gly-Pro-Arg-pNA). Similar data was obtained using either substrate. The microplate was then shaken on a platform vortex mixer at a setting of 800 (Sarstedt TPM-2). After a total of three minutes incubation, 10 μl of the working stock solution of tryptase (6.1 mM final tryptase concentration for use with BAPNA or 0.74 nM for use with CBz-Gly-Pro-Arg-pNA) was added to each well. The microplate was vortexed again for one minute and then incubated without shaking at room temperature for an additional 2 minutes. After this time the microplate was read on a microplate reader (Molecular Devices WV max) in the kinetic mode (405 nm wavelength) over twenty minutes at room temperature. To determine the compound concentration that inhibited half of the enzyme activity (IC₅₀), the fraction of control activity (FCA) was plotted as a function of the inhibitor concentration (I) and curve to fit FCA/(1+[I]/IC₅₀). The IC₅₀ for each compound was determined 2-4 times and the obtained values were averaged.

As a result of this tryptase activity, the compounds of formula I to Il as well as an inner salt thereof, a pharmaceutically acceptable salt thereof, a hydrolyzable ester thereof, or a solvate thereof, are useful as antiinflammatory agents particularly in the treatment of chronic asthma and may also be useful in treating or preventing allergic rhinitis, inflammatory bowel disease, psoriasis, conjunctivitis, atopic dermatitis, rheumatoid arthritis, osteoarthritis, and other chronic inflammatory joint diseases, or diseases of joint cartilage destruction. Additionally, these compounds may be useful in treating or preventing myocardial infarction, stroke, angina and other consequences of atherosclerotic plaque rupture. Additionally, these compounds may be useful for treating or preventing diabetic retinopathy, tumor growth and other consequences of angiogenosis. Additionally, these compounds may be useful for treating or preventing fibrotic conditions, for example, fibrosis, scleroderma, pulmonary fibrosis, liver cirrhosis, myocardial fibrosis, neurofibromas and hypertrophic scars.

The compounds of formula I to Il are also inhibitors of Factor Xa and/or Factor Vila. As a result, the compounds of formula I to VI as well as an inner salt or a pharmaceutically acceptable salt thereof, a hydrolyzable ester thereof, or a solvate thereof may also be useful in the treatment or prevention of thrombotic events associated with coronary artery and cerebrovascular disease which include the formation and/or rupture of atherosclerotic plaques, venous or arterial thrombosis, coagulation syndromes, ischemia and angina (stable and unstable), deep vein thrombosis (DVT), disseminated intravascular coagulopathy, Kasacach-Merritt syndrome, pulmonary embolism, myocardial infarction, cerebral infarction, cerebral thrombosis, transient ischemic attacks, atrial fibrillation, cerebral embolism, thromboembolic complications of surgery (such as hip or knee replacement, introduction of artificial heart valves and endarterectomy) and peripheral arterial occulsion and may also be useful in treating or preventing myocardial infarction, stroke, angina and other consequences of atherosclerotic plaque rupture. The compounds of formula I to Il possessing Factor Xa and/or Factor VIIa inhibtion activity may also be useful as inhibitors of blood coagulation such as during the preparation, storage and fractionation of whole blood.

The compounds of formula I to Il are also inhibitors of urokinase-type plasminogen activator. As a result, the compounds of formula I to Il as well as an inner salt or a pharmaceutically acceptable salt thereof, a hydrolyzable ester thereof, or a solvate thereof may be useful in the treatment or prevention of restenosis and aneurysms, in the treatment or prevention of myocardial infarction, stroke, angina and other consequences of atherosclerotic plaque rupture, and may also be useful in the treatment of malignancies, prevention of metastases, prevention of prothrombotic complications of cancer, and as an adjunct to chemotherapy.

The compounds of formulas I to Il also possess thrombin and trypsin inhibitory activity similar to that reported by Han in the U.S. patents noted previously for the compounds of formula I to Il. As a result, the compounds of formula I to Il as well as an inner salt or a pharmaceutically acceptable salt thereof, a hydrolyzable ester thereof, or a solvate thereof may be useful in treating or preventing pancreatitis, in the treatment or prevention of thrombotic events associated with coronary artery and cerebrovascular disease as described above, and may also be useful as inhibitors of blood coagulation such as during the preparation, storage, and fractionation of whole blood.

Certain compounds of formulas I to Il are also useful due to their selective tryptase inhibition activity. These compounds while having potent tryptase inhibition activity are much less active against other enzyme systems including trypsin, thrombin and Factor Xa. For example, this selective tryptase activity is seen with the compounds of formulas I to Il where X₁ is the group

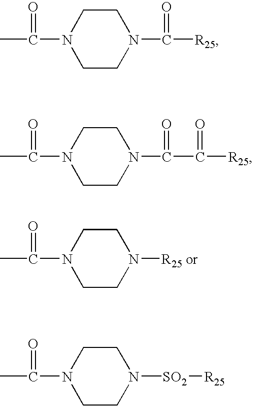

and R₂₅ is a spacer terminating in a lipophilic group. Suitable spacers include groups of 3 or more atoms such as

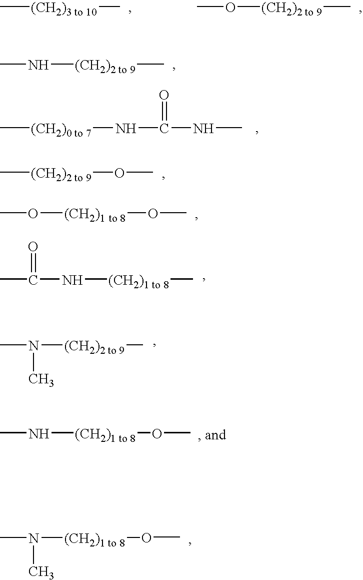

etc., as well as groups containing 2 or more atoms and a phenyl, substituted phenyl, cycloalkyl, heteroaryl, or heterocycloalkyl ring such as

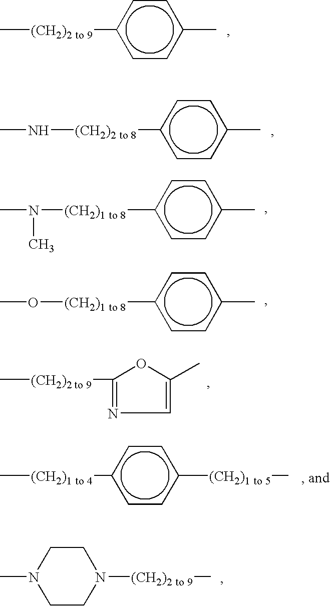

etc. Suitable lipophilic terminal groups include aryl, substituted aryl, cycloalkyl, heteroaryl, heterocycloalkyl, etc. These compounds of formulas I to Il as well as an inner salt, a pharmaceutically acceptable salt thereof, a hydrolyzable ester thereof, or a solvate thereof, are useful as anti-inflammatory agents particularly in the treatment of chronic asthma and may also be useful in treating or preventing allergic rhinitis as well as some of the other diseases described above for the non-selective tryptase inhibitors. It is believed that as a result of their selective tryptase inhibition activity that these compounds will have less tendency to produce unwanted side-effects.

The compounds of formula I to Il as well as an inner salt or a pharmaceutically acceptable salt thereof, a hydrolyzable ester thereof, or a solvate thereof may be administered orally, topically, rectally or parenterally or may be administered by inhalation into the bronchioles or nasal passages. The method of administration will, or course, vary upon the type of disease being treated. The amount of active compound administered will also vary according to the method of administration and the disease being treated. An effective amount will be within the dosage range of about 0.1 to about 100 mg/kg, preferably about 0.2 to about 50 mg/kg and more preferably about 0.5 to about 25 mg/kg per day in a single or multiple doses administered at appropriate intervals throughout the day.

The composition used in these therapies can be in a variety of forms. These include, for example, solid, semi-solid and liquid dosage forms such as tablets, pills, powders, liquid solutions or suspensions, liposomes, injectable and infusible solutions. Such compositions can include pharmaceutically acceptable carriers, preservatives, stabilizers, and other agents conventionally employed in the pharmaceutical industry.

When the compounds of formula I to Il as well as an inner salt or a pharmaceutically acceptable salt thereof, a hydrolyzable ester thereof, or a solvate thereof are employed to treat asthma or allergic rhinitis they will preferably be formulated as aerosols. The term “aerosol” includes any gas-borne suspended phase of the active compound which is capable of being inhaled into the bronchioles or nasal passage. Aerosol formulations include a gas-borne suspension of droplets of the active compound as produced in a metered dose inhaler or nebulizer or in a mist sprayer. Aerosol formulations also include a dry powder composition suspended in air or other carrier gas. The solutions of the active compounds of formulas I to Il used to make the aerosol formulation will be in a concentration of from about 0.1 to about 100 mg/ml, more preferably 0.1 to about 30 mg/ml, and most preferably from about 1 to about 10 mg/ml. The solution will usually include a pharmaceutically acceptable buffer such as a phosphate or bicarbonate to give a pH of from about 5 to 9, preferably 6.5 to 7.8, and more preferably 7.0 to 7.6. Preservatives and other agents can be included according to conventional pharmaceutical practice.

Other pharmaceutically active agents can be employed in combination with the compounds of formula I to Il depending upon the disease being treated. For example, in the treatment of asthma, β-adrenergic agonists such as albuterol, terbutaline, formoterol, fenoterol or prenaline can be included as can anticholinergics such as ipratropium bromide, anti-inflammatory cortiocosteroids such as beclomethasone, triamcinolone, flurisolide or dexamethasone, and anti-inflammatory agents such as cromolyn and nedocromil.

The following abbreviations are employed herein.

PVP=polyvinylpyrrolidone

Ph=phenyl

Bn=benzyl

BnOH=benzyl alcohol

t-Bu=tertiary butyl

Me=methyl

Et=ethyl

TMS=trimethylsilyl

TMS-NCO=trimethylsilylisocyanate

TMSN₃=trimethylsilyl azide

TBS=tert-butyldimethylsilyl

FMOC=fluorenylmethoxycarbonyl

Boc=tert-butoxycarbonyl

Cbz=carbobenzyloxy or carbobenzoxy or benzyloxycarbonyl

THF=tetrahydrofuran

OSu=N-oxysuccinimide

Et₂O=diethyl ether (ether)

hex=hexanes

EtOAc=ethyl acetate

DMF=dimethyl formamide

MeOH=methanol

EtOH=ethanol

i-PrOH=isopropanol

DMSO=dimethyl sulfoxide

DME=1,2 dimethoxyethane

DCE=1,2 dichloroethane

HMPA=hexamethyl phosphoric triamide

HOAc or AcOH=acetic acid

TFA=trifluoroacetic acid

TFAA=trifluoroacetic anhydride

i-Pr₂NEt=diisopropylethylamine

Et₃N=triethylamine

NMM=N-methyl morpholine

DMAP=4-dimethylaminopyridine

NaBH₄=sodium borohydride

NaBH(OAc)₃=sodium triacetoxyborohydride

DIBALH=diisobutyl aluminum hydride

DIPEA=diisopropylethyl amine

TEA=triethylamine

DCM=4-(dicyanomethylene)-2-methyl-6-(4-dimethylaminostyryl)-4H-pyran

NBS=N-bromosuccinimide

NaHMDS=sodium hexamethyldisilazide

DCC=1,3-dicyclohexylcarbodiimide

LAH or LiAlH₄=lithium aluminum hydride

n-BuLi=n-butyllithium

LDA=lithium diisopropylamide

Pd/C=palladium on carbon

PtO₂=platinum oxide

KOH=potassium hydroxide

NaOH=sodium hydroxide

LiOH=lithium hydroxide

K₂CO₃=potassium carbonate

NaHCO₃=sodium bicarbonate

DBU=1,8-diazabicyclo[5.4.0]undec-7-ene

EDC (or EDC.HCl) or EDCI (or EDCI.HCl) or EDAC=3-ethyl-3′-(dimethylamino)propyl-carbodiimide hydrochloride (or 1-(3-dimethylaminopropyl)-3-ethylcarbodiimide hydrochloride)

HOAt=1-Hydroxy-7-azabenzotriazole

Ph₃P=triphenylphosphine

Pd(OAc)₂=Palladium acetate

Cbz-Cl=benzyl chloroformate

Ar=argon

N₂=nitrogen

min=minute(s)

h or hr=hour(s)

L=liter

mL=milliliter

μL=microliter

g=gram(s)

mg=milligram(s)

mol=moles

mmol=millimole(s)

meq=milliequivalent

RT=room temperature

sat or sat'd=saturated

aq.=aqueous

TLC=thin layer chromatography

HPLC=high performance liquid chromatography

LC/MS=high performance liquid chromatography/mass spectrometry

MS or Mass Spec=mass spectrometry

NMR=nuclear magnetic resonance

NMR spectral data: s=singlet; d=doublet; m=multiplet; br=broad; t=triplet

mp=melting point

### EXAMPLES

The following Examples represent preferred embodiments of the invention.

### Example 1

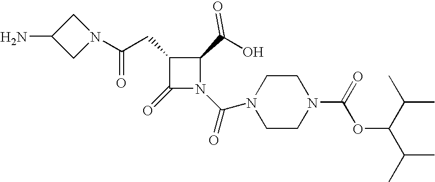

The title compound was prepared according to the following reaction sequence and as described below.

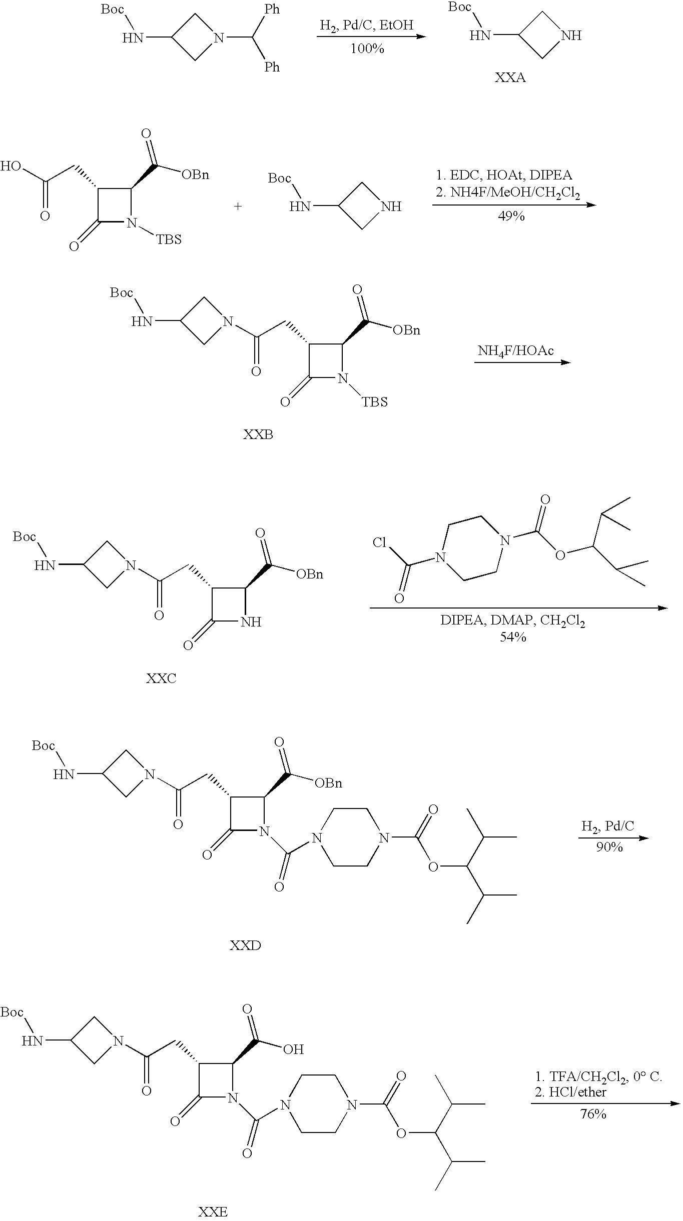

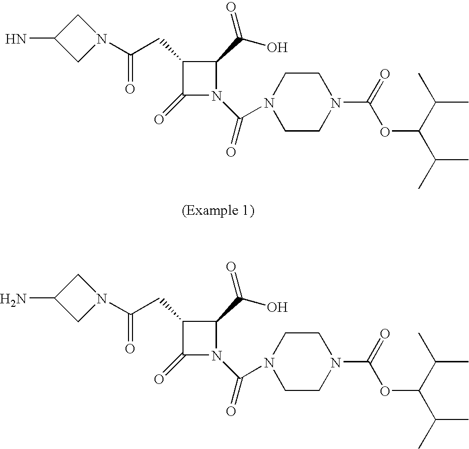

### (Example 1)

### Synthesis of Example 1 Compound

1. Step A

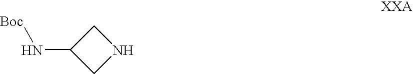

A mixture of

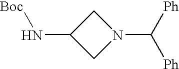

(113 mg, 0.33 mmol) and 10% Pd/C (88 mg) in ethanol (3 ml) was stirred under hydrogen atmosphere at rt for 2 h. The reaction mixture was filtered and evaporated to give XXA (63 mg) as a white solid.

2. Step B

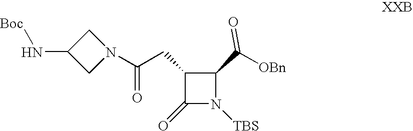

A mixture of

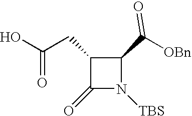

(101 mg, 0.27 mmol), XXA (57 mg), EDC (51 mg, 0.27 mmol) and (37 mg, 0.27 mmol) in DCM (2 ml) was stirred at rt for 3 h. The reaction was quenched with the addition of brine (10 ml). The mixture was then extracted with DCM (30 ml). The extract was dried (MgSO₄) and evaporated to give the crude product which was purified by flash chromatography (silica, 0-4% MeOH/DCM) to give XXB (88 mg) as a colorless oil.

3. Step C

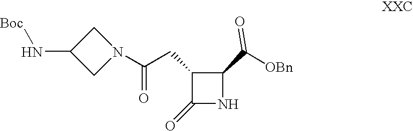

A solution of XXB (88 mg) was dissolved in DCM (1 ml) and the solution was treated with HOAc (20 μl) and NH₄F (0.5 M in MeOH, 340 μl) for 5 min. The mixture was concentrated and the residue was purified by flash chromatography (silica, 2-5% MeOH/DCM) to give XXC (55 mg) as a yellow foam.

4. Step D

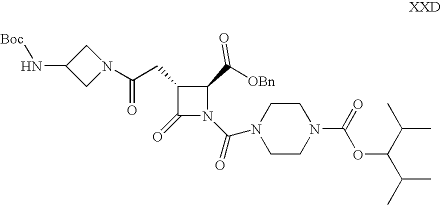

A mixture of XXC (52 mg, 0.12 mmol),

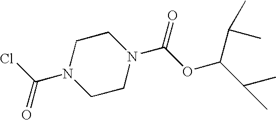

(42 mg, 0.14 mmol), DIPEA (24 μl, 0.14 mmol) and a few crystals of DMAP in DCM (1.5 ml) was stirred at rt for 4 h. The reaction was quenched with the addition of brine and extracted with DCM. The extract was dried (MgSO₄) and evaporated to give the crude product which was purified by flash chromatography (silica, 0-4% MeOH/DCM) to give XXD (44 mg) as a colorless solid.

5. Step E

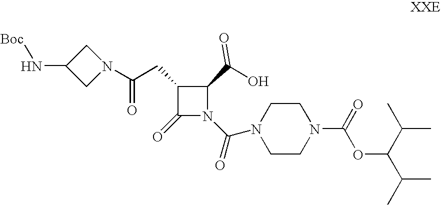

A mixture of XXD (41 mg, 0.061 mmol) and 10% Pd/C (15 mg) in ethanol (1 ml) was stirred under hydrogen atmosphere at rt for 1 h. The reaction mixture was filtered and evaporated to give XXE (32 mg).

6. Step F (Example 1)

A solution of XXE (31 mg, 0.053 mmol) in DCM (0.75 ml) was cooled to −5° C. and treated with TFA (250 μl). The mixture was stirred at 0° C. for 5 min and then treated with HCl (53 μl, 1 N in Et₂O). The reaction was stirred at 0° C. until completion. The mixture was evaporated and the residue was dissolved in water to give Example 1 compound (27 mg) as a white solid.

### Example 2

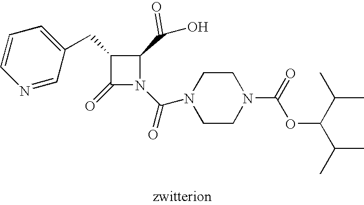

The title compound was prepared according to the following reaction scheme and as described below.

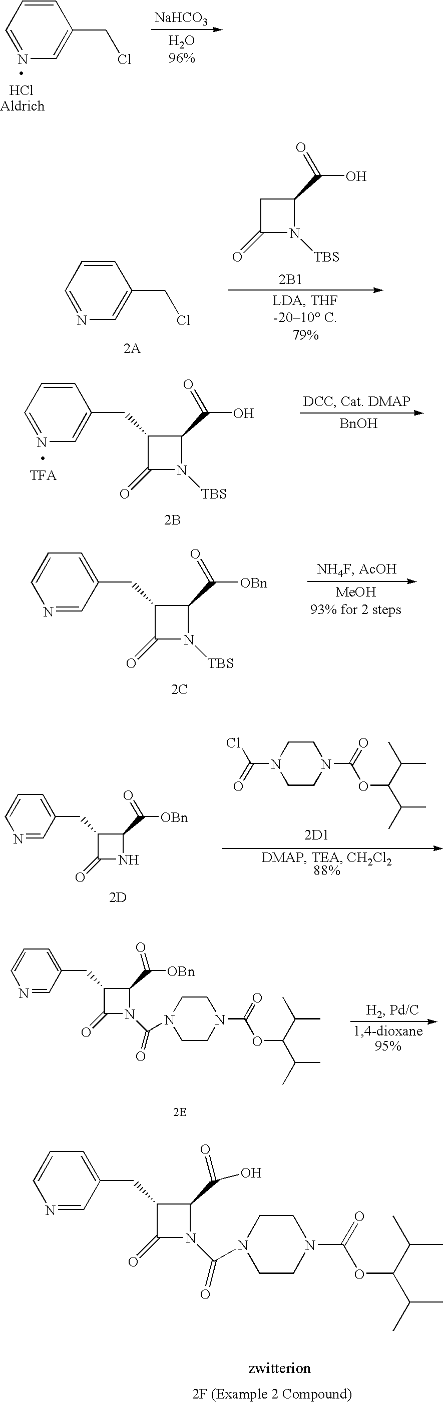

### Synthesis of Example 2 Compound

1. Preparation of 2A

To a solution of 3-picolyl chloride hydrochloride (1.00 g, 6.10 mmol) in 10 mL of distilled water was added NaHCO₃ (0.77 g, 9.14 mmol) with stirring. The mixture was extracted with Et₂O (20 mL×3). The extracts were combined, washed with brine, dried over MgSO₄, and concentrated to give the free amine 2A (0.76 g) as a colorless oil.

2. Preparation of 2B To a solution of diisopropylamine (0.68 mL, 4.82 mmol) in 5 mL of THF was added BuLi (1.6 M, 2.88 mL, 4.62 mmol) at −15° C. The solution was cooled to −78° C. and stirred for 20 minutes. A solution of 2β-lactam B1 (500 mg, 2.20 mmol) in 2 mL of THF was added dropwise. The solution was warmed to −20° C. to −10° C. and stirred at this temperature for 30 minutes. Then a solution of 3-picolyl chloride (2A) (420 mg, 3.30 mmol) in 3 mL of THF was added. The reaction mixture was stirred for 2 hours and then quenched with TFA (0.17 mL, 2.20 mmol). THF was removed and the residue was purified by preparative HPLC (reverse phase, methanol, water, TFA) to provide (after lyophilization) alkylation product 2B (568 mg) as a white solid. MS 321.4 (M+H)⁺.

3. Preparation of 2C

To a mixture of acid 2B (290 mg 0.67 mmol) and benzyl alcohol (0.21 mL, 2.00 mmol) was added DCC (206 mg, 1.00 mmol) and DMAP (16 mg, 0.13 mmol) at 0° C. and stirred for 5 minutes. Then ice bath was removed. The mixture was stirred at rt for 3 hours. 15 mL of CH₂Cl₂ was added. The precipitate was filtered and washed with CH₂Cl₂ (10 mL×2). The CH₂Cl₂ solution was concentrated to crude 2C.

4. Preparation of 2D

The crude 2C was dissolved in 5 mL of methanol. Acetic acid (0.12 mL, 2.14 mmol) and a solution of NH₄F (0.5 M, 1.3 mL, 0.67 mmol) in methanol was added. The solution was stirred for 1 hour. The solvents were replaced with 30 mL of CH₂Cl₂, washed with sat. NaHCO₃ solution (5 mL) and brine (5 mL), dried over MgSO₄, and concentrated. The residue was purified with silica gel chromatography (ethyl acetate:hexane=4:1, Rf=0.25) to afford 2D (200 mg) as a white solid. MS 297.3 (M+H)⁺.

5. Preparation of 2E

To a solution of ester 2D (150 mg, 0.51 mmol) in 4 mL of CH₂Cl₂ was added triethylamine (0.085 mL, 0.61 mmol), acid chloride 2D1 (176 mg, 0.61 mmol), and DMAP (4.0 mg, 0.031 mmol). The solution was stirred for 3 hour. The solvent was removed. The residue was purified with silica gel chromatography (ethyl acetate:hexane=2:1, Rf=0.22) to afford 2E (245 mg) as a white solid. MS 551.2 (M+H)⁺.

6. Preparation of 2F (Example 2 Compound)

A mixture of 2E (100 mg, 0.18 mmol), Pd/C (10%, 60 mg) in dioxane (2 mL) was stirred under hydrogen atmosphere (hydrogen balloon) at room temperature for 3 hours. Analytical HPLC indicated the completion of the reaction. The reaction mixture was filtered through a celite pad, lyophilized to give Example 2 compound (79 mg, zwitterion) as a white foam. Purity by anal HPLC: 100%; MS: (M+H)⁺ 461.2, (M−H)⁻ 459.2; IR (KBr pellet) ν 1785 cm⁻¹.

### Example 3

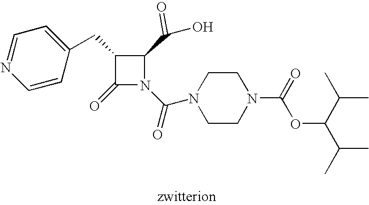

The title compound was prepared according to the following reaction sequence and as described below.

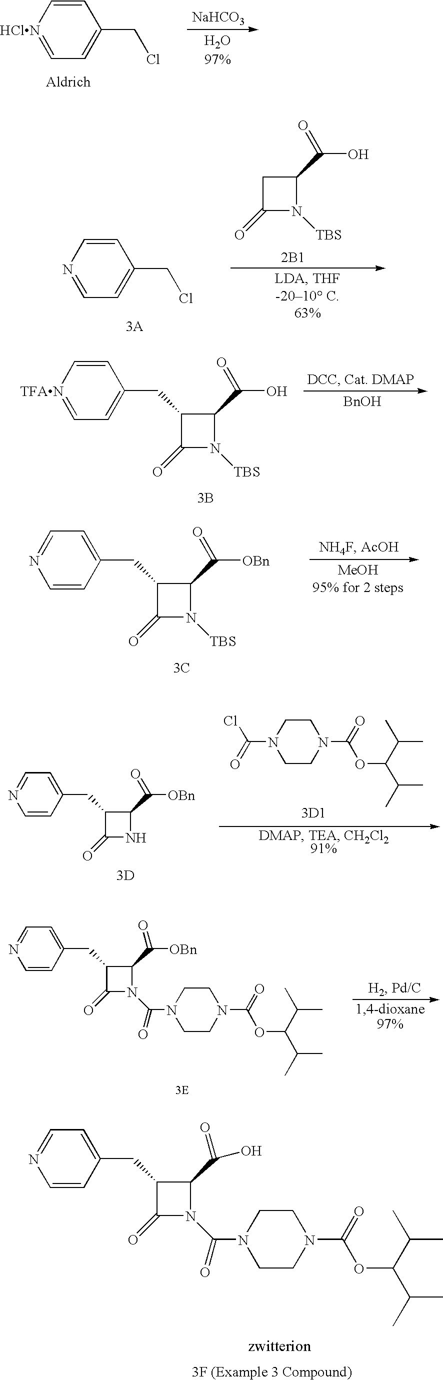

### Synthesis of Example 3 Compound

1. Preparation of 3A

To a solution of 4-picolyl chloride hydrochloride (1.00 g, 6.10 mmol) in 10 mL of distilled water was added NaHCO₃ (0.77 g, 9.14 mmol) with stirring. The mixture was extracted with Et₂O (20 mL×3). The extracts were combined, washed with brine, dried over MgSO₄, and concentrated to give the free amine 3A (0.75 g) as a colorless oil.

2. Preparation of 3B

To a solution of diisopropylamine (0.68 mL, 4.82 mmol) in 5 mL of THF was added BuLi (1.6 M, 2.88 mL, 4.62 mmol) at −15° C. The solution was cooled to −78° C. and stirred for 20 minutes. A solution of β-lactam 2B1 (500 mg, 2.20 mmol) in 2 mL of THF was added dropwise. The solution was warmed to −20° C. to −10° C. and stirred at this temperature for 30 minutes. Then a solution of 4-picolyl chloride (3A) (420 mg, 3.30 mmol) in 3 mL of THF was added. The reaction mixture was stirred for 2 hours and then quenched with TFA (0.17 mL, 2.20 mmol). THF was removed and the residue was purified by preparative HPLC (reverse phase, methanol, water, TFA) to provide (after lyophilization) alkylation product 3B (453 mg) as white solid. MS 321.4 (M+H)⁺.

3. Preparation of 3C

To a mixture of acid 3B (310 mg 0.71 mmol) and benzyl alcohol (0.23 mL, 2.14 mmol) was added DCC (309 mg, 1.50 mmol) and DMAP (17 mg, 0.14 mmol) at 0° C. and stirred for 5 minutes. Then ice bath was removed. The mixture was stirred at rt for 3 hours. 15 mL of CH₂Cl₂ was added. The precipitate was filtered and washed with CH₂Cl₂ (10 mL×2). The CH₂Cl₂ solution was concentrated to crude 3C.

4. Preparation of 3D

The crude 3C was dissolved in 5 mL of methanol. Acetic acid (0.13 mL, 2.14 mmol) and a solution of NH₄F (0.5 M, 1.4 mL, 0.71 mmol) in methanol was added. The solution was stirred for 1 hour. The solvents were replaced with 30 mL of CH₂Cl₂, washed with sat. NaHCO₃ solution (5 mL) and brine (5 mL), dried over MgSO₄, and concentrated. The residue was purified with silica gel chromatography (ethyl acetate:hexane=4:1, Rf=0.25) to afford 3D (200 mg) as a white solid. MS 297.3 (M+H)⁺.

5. Preparation of 3E

To a solution of ester 3D (120 mg, 0.41 mmol) in 4 mL of CH₂Cl₂ was added triethyl amine (0.068 mL, 0.49 mmol), acid chloride 3D1 (143 mg, 0.49 mmol), and DMAP (2.5 mg, 0.021 mmol). The solution was stirred for 3 hour 3 hour. The solvent was removed. The residue was purified with silica gel chromatography (ethyl acetate:hexane=2:1, Rf=0.22) to afford 3E (205 mg) as a white solid. MS 551.2 (M+H)⁺.

6. Preparation of 3F (Example 3 Compound)

A mixture of 3E (100 mg, 0.18 mmol), Pd/C (10%, 60 mg) in dioxane (2 mL) was stirred under hydrogen atmosphere (hydrogen balloon) at room temperature for 3 hours. Analytical HPLC indicated the completion of the reaction. The reaction mixture was filtered through a celite pad, lyophilized to give Example 3 compound (80 mg, zwitterion) as a white foam. Purity by anal HPLC: 96%; MS: (M+H)⁺ 461.2, (M−H)⁻ 459.2; IR (KBr pellet) ν 1785 cm⁻¹.

### Example 4

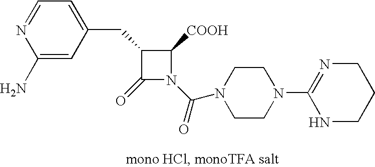

The title compound was prepared according to the following reaction scheme and as described below.

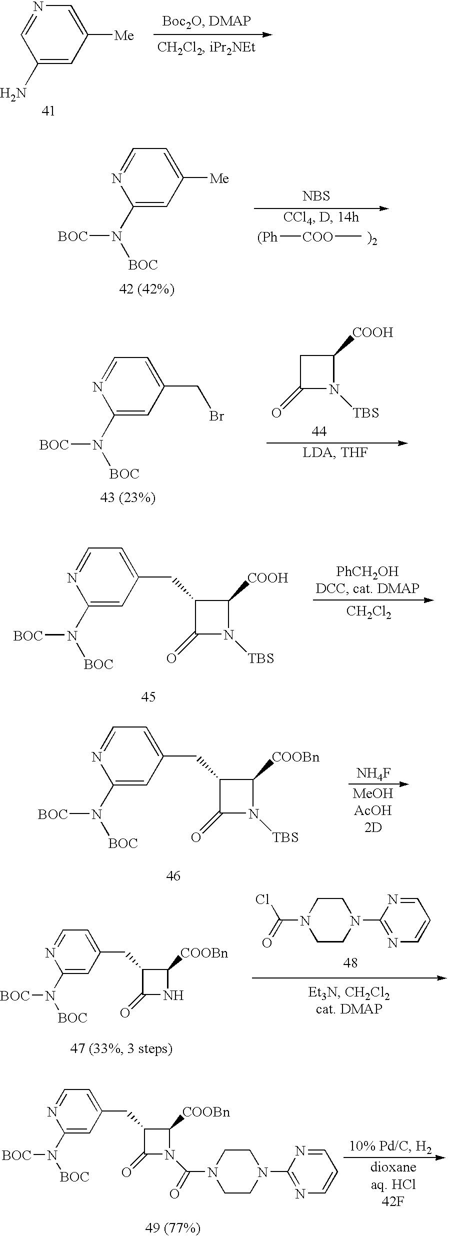

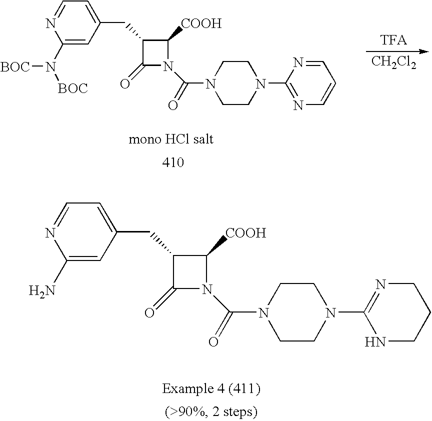

### Synthesis of Example 4 Compound

1. Preparation of 42

To a solution of 2-amino-4-picoline (41) (5.41 g, 50 mmol) in dichloromethane (100 mL) at 0° C. was added N,N-diisopropylethylamine (17.4 mL, 100 mmol), di-t-butyldicarbonate (27.3 g, 125 mmol), and DMAP (6.1 g, 50 mmol). After the addition, the reaction was stirred at room temperature for 16 hours and diluted with ethyl acetate. The organics were washed with saturated aqueous ammonium chloride (3×), brine (1×), saturated sodium bicarbonate (2×) and brine, dried (magnesium sulfate) and concentrated to a viscous oil (14.3 g). Purification of a 7.5 g portion of this oil over silica gel using dichloromethane-ethyl acetate (19:1) afforded 3.32 g of compound 42 as a solid.

2. Preparation of 43

A mixture of compound 42 (1.85 g, 6 mmol), carbon tetrachloride (30 mL), N-bromosuccinimide (NBS) (1.07 g, 6 mmol), and benzoyl peroxide (145 mg, 0.6 mmol) was refluxed for 14 hours, cooled, and filtered. Evaporation of the filtrate and concentration of the residue from dichloromethane (4×) gave an oil (2.37 g), which was flash chromatographed over silica gel using dichloromethane-ethyl acetate (19:1) to provide 534 mg of compound 43 as an oily residue.

3. Preparation of 45

To 0.32 mL (2.28 mmol) of diisopropylamine in 2 mL of tetrahydrofuran at −20° C. under argon was added 0.8 mL of 2.5 M n-butyl lithium in hexane (2 mmol). The mixture was stirred for 10 minutes and cooled to −70° C. A solution of 229 mg (1.0 mmol) of compound 44 in 2 mL of tetrahydrofuran was added over 3 minutes and the reaction was warmed to −20° C. over 15 minutes. A solution of 526 mg (1.36 mmol) of compound 3 in 3 mL of tetrahydrofuran was added and the reaction was stirred between −20° C. and −30° C. for 5 hours and then stored at −40° C. for 16 hours. The reaction was warmed to 0° C. and quenched by addition of 10% potassium hydrogensulfate (5.5 mL) and then water and ethyl acetate. After a total of 3 extractions with ethyl acetate, the ethyl acetate was washed with brine, dried (sodium sulfate), and concentrated to give 730 mg of crude compound 45 as an oily residue.

4. Preparation of 46

A mixture of 540 mg of crude compound 45 above, dichloromethane (1.4 mL), benzyl alcohol (168 μL, 1.62 mmol), N,N′-dicyclohexylcarbodiimide (223 mg, 1.08 mmol), and DMAP (18 mg, 0.15 mmol) was stirred at room temperature for 5 hours and stored overnight at 5° C. The solids were filtered off and the filtrate was concentrated to a residue, which was taken up in ethyl acetate. After 20 minutes, solids were removed by filtration and the filtrate was concentrated to give 732 mg of crude compound 46 as an oil.

5. Preparation of 47

A solution of 2.2 mL of 0.5 M ammonium fluoride in methanol (1.1 mmol) was added to a mixture of 732 mg of crude compound 46 above, methanol (5 mL), and acetic acid (190 μL, 3.3 mmol) and the mixture was stirred at room temperature for 1.5 hours and then concentrated to a residue. The residue was taken up in ethyl acetate and water and the pH was adjusted to 8.7 with aqueous 5% sodium bicarbonate. The layers were separated and the aqueous layer was extracted with ethyl acetate. The combined organic layers were washed with brine (2×), dried (sodium sulfate) and concentrated to an oily solid. Ethyl acetate was added and after filtration the filtrate was concentrated to an oily residue (558 mg). Chromatography of the residue over silica gel using dichloromethane-ethyl acetate (75:25) afforded 125 mg of compound 47 as a residue.

6. Preparation of 49

A mixture of compound 47 (58 mg, 0.11 mmol), dichloromethane (1.5 mL), compound 48 (30 mg, 0.13 mmol), triethylamine (35 μL, 0.25 mmol), and DMAP (3 mg, 0.025 mmol) was stirred at room temperature for 16 hours. The reaction was concentrated and the residue was taken up in ethyl acetate, water, and several drops of 10% potassium hydrogensulfate. The pH was adjusted to 8.0 (aqueous sodium bicarbonate) and the ethyl acetate layer was separated and washed with water (2×), dried (sodium sulfate), and concentrated to an oil (84 mg). Chromatography of the oil over silica gel using dichloromethane-ethyl acetate (60:40) gave 59 mg of compound 49 as a residue.

7. Preparation of 410

Compound 49 (57 mg, 0.081 mmol) was hydrogenated at 1 atmosphere in dioxane (4 mL) and 82 μL of aq. 1.0 N HCl (0.082 mmol), in the presence of 19 mg of 10% palladium on carbon for 2 hours. The reaction was filtered with aqueous dioxane and the filtrate was concentrated and lyophilized to give 49 mg of compound 410 as a white powder.

8. Preparation of 411 (Example 4 Compound)

Trifluoroacetic acid (0.15 mL) was added to a stirred solution of compound 410 (45 mg) in dichloromethane (2 mL) at 0-5° C. The reaction was stirred at ambient temperature for 1.5 hours and 0.5 mL additional trifluoroacetic acid was added. After 1.5 hours, dioxane (5 mL) was added and the solution was concentrated and lyophilized from aqueous dioxane to give 44 mg of compound 411 (Example 4 compound) as a white solid; IR (KBr) 1789 cm⁻¹; mass spectrum (M+H)⁺ 416.

### Example 5

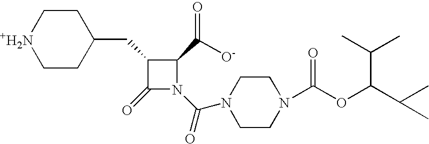

The title compound was prepared according to the following reaction scheme and as described below.

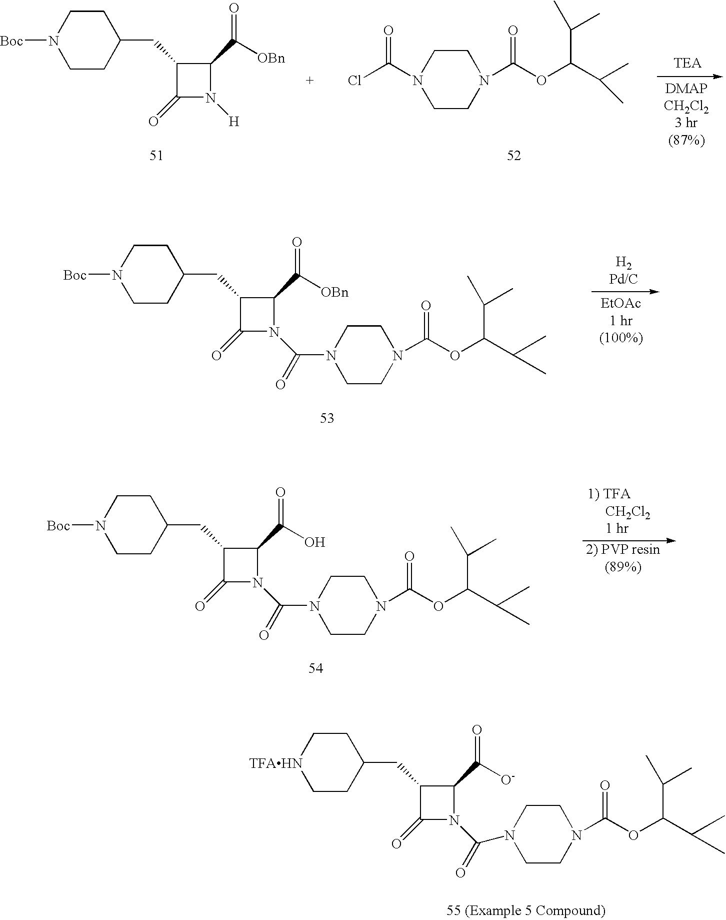

### Preparation of Example 5 Compound

1. Preparation of 51

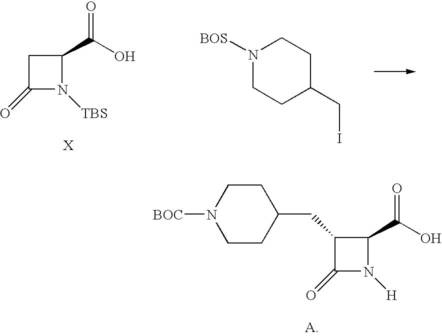

A. To a solution of iPr₂NH (6.42 ml, 45.8 mmol) in THF (15 ml) at 0° C. was added nBuLi (1.6 M, 28 ml, 45 mmol). After stirring at room temperature for 1 hour, the reaction mixture was cooled to −78° C. and treated dropwise over 5 minutes with a solution of X (5 g, 21.8 mmol in 15 ml THF). The reaction mixture was then warmed to −20° C. and a slurry formed. THF (15 ml) was added to loosen the slurry and the slurry was stirred vigorously for 2.5 hour at −20° C. A solution of N-Boc-4-(iodomethyl)piperidine (10.63 g, 32.7 mmol in 15 ml THF) was added dropwise over 3 minutes and the resulting solution was stirred at −20° C. for 18 hours. The reaction mixture was partitioned between EtOAc (200 ml) and water (200 ml). After mixing and separation, the organic phase was extracted once more with water (150 ml). The combined water layers were then washed with EtOAc (2×200 ml). The aqueous phase was diluted with EtOAc (200 ml) and with rapid stirring brought to pH 2-3 with portionwise addition of solid KHSO₄. The EtOAc layer was removed and the aqueous layer was extracted with EtOAc (3×200 ml). The EtOAc extracts from the acidified aqueous layer were combined, dried over Na₂SO₄, filtered and concentrated.

The residue was taken up in THF (30 ml), treated with TBAF (1.0 M THF, 22 ml) and stirred at room temperature for 2 hours. The reaction mixture was partitioned between EtOAc and 0.5N KHSO4. The aqueous phase was then extracted with EtOAc (4×). The combined EtOAc extracts were dried over MgSO4, filtered and concentrated to give Part A compound (2.8 g, 42%).

1H-NMR (400 MHz, CDCl3) δ 8-9 (bs, 1H), 6.82 (s, 1H), 4.1 (m, 2H), 3.88 (d, 1H, j=2.47), 3.36 (m, 1H), 2.67 (m, 2H), 1.6-1.9 (m, 6H), 1.43 (s, 9H), 1.1 (m, 1H).

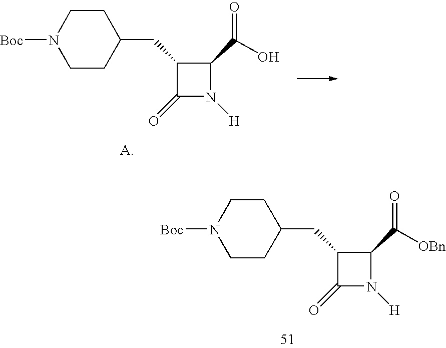

B. NaHCO₃ (0.58 g, 6.92 mmol) was added to a stirred solution of crude Part A compound (1.08 g, 3.46 mmol) and benzylbromide (2.06 ml, 17.30 mmol) in DMF (10 ml) at room temperature. After 20 h the reaction mixture was partitioned between ethylacetate and water. The organic phase was isolated, washed with saturated NaCl, dried over MgSO₄, filtered and concentrated. The residue was purified by silica gel chromatography to afford 0.77 g of compound 51.

2. Preparation of 52

H₂O (2.0 mL) was added to compound 51 (20 mg, 0.043 mmol). MeOH was added until all of 51 dissolved. 1N HCl (45 μL, 0.045 mmol) was added. After 5 min of stirring the solution was conc. to remove MeOH and lyophilized. 18 mg (84%) of 52 was obtained. IR (KBr): 1788 cm⁻¹

3. Preparation of 53

Compound 51 (244 mg, 0.606 mmol) and Compound 52 (212 mg, 0.727 mmol) were dissolved in CH₂Cl₂ (3.0 mL). TEA (127 uL, 0.909 mmol) was added followed by DMAP (15 mg, 0.121 mmol). After 24 hr the reaction mixture was conc. and the residue was partitioned between EtOAc and H₂O. The organic phase was isolated, washed with 1N HCl, sat. NaCl, dried (MgSO₄), and conc. The residue was purified by silica gel chromatography to afford 348 mg (87%) of 53.

4. Preparation of 54

Compound 53 (138 mg, 0.210 mmol) was dissolved in EtOAc (0.40 mL). 10% Pd/C (15 mg) was added and a H₂ atmosphere was introduced via balloon. After 1 hr the reaction mixture was diluted with EtOAc and filtered. The filtrate was conc. to afford 122 mg (100%) of 54.

5. Preparation of 55 (Example 55 Compound)

TFA (0.40 mL) was added dropwise to a stirred solution of compound 54 (86 mg, 0.152 mmol) in CH₂Cl₂ (1.20 mL) at 0° C. The reaction mixture was then stirred at room temp. After 1 hr the reaction mixture was conc. and placed under vacuum. The crude product was dissolved in H₂O and placed on top of a column of cleaned PVP resin. The column was eluted with H₂O. Fractions containing product were combined and lyophilized. 63 mg (89%) of 55 (Example 5 compound) was obtained. LR-MS (ESI); (M+H) calc'd=467, found=467.

### Example 6

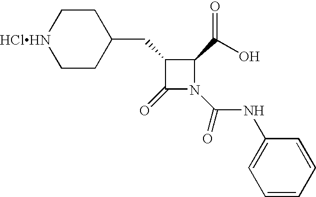

The title compound was prepared according to the following reaction scheme as described below.

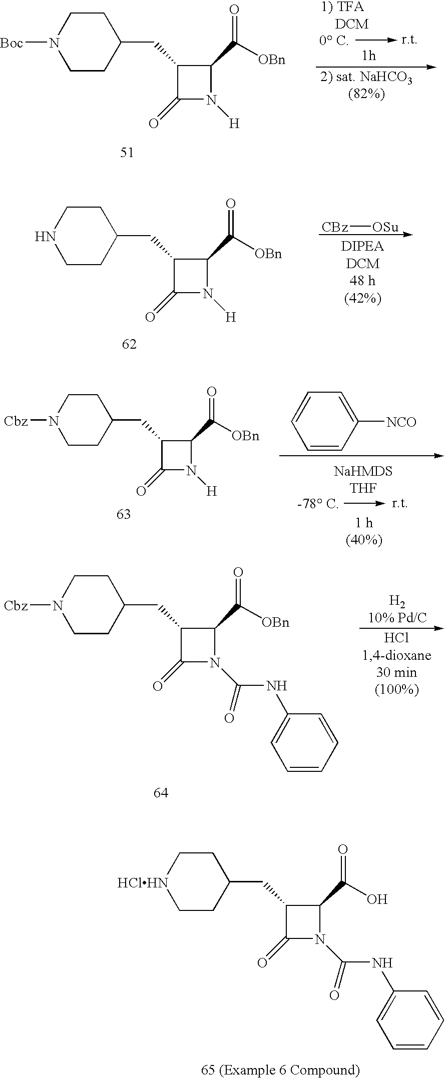

### Preparation of Example 6 Compound

1. Preparation of 62

TFA (0.40 mL) was added dropwise to a stirred solution of compound 51 prepared in Example 5 (130 mg, 0.323 mmol) in CH₂Cl₂ (1.20 mL) at 0° C. The reaction mixture was then stirred at room temp. After 1 hr the reaction mixture was conc. and placed under vacuum. The crude product was stirred with CHCl₃ and sat. NaHCO₃. The organic phase was isolated, dried (MgSO₄), and conc. 80 mg (82%) of 62 was obtained.

2. Preparation of 63

N-(Benzyloxycarbonyloxy)succinimide (61 mg, 0.243 mmol) was added to a stirred solution of compound 62 (70 mg, 0.171 mmol) in CH₂Cl₂ (1.00 mL). After 24 hr DIPEA (40 μL, 0.232 mmol) was added. After 24 hr the reaction mixture was diluted with EtOAc, washed with 1N HCl, sat. NaHCO₃ and sat. NaCl. dried (MgSO₄), and conc. The crude product was purified by silica gel chromatography to give 42 mg (42%) of compound 63.

3. Preparation of 64

A 1.0M THF solution of NaHMDS (115 μL, 0.115 mmol) was added dropwise to a stirred solution of compound 63 (42 mg, 0.096 mmol) in THF (0.70 mL) at −78° C. After 25 min of stirring phenyl isocyanate (12 μL, 0.106 mmol) was added dropwise. The temp. was slowly raised to room temp. After 1 h the reaction mixture was cooled to 0° C. quenched by addition of 5% KHSO₄. The solution was partitioned between EtOAc and water. The organic phase was isolated, washed with sat. NaCl, dried (MgSO₄), and conc. The residue was purified by silica gel chromatography to afford 21 mg (40%) of compound 64.

4. Preparation of 65 (Example 6 Compound)

Compound 64 (21 mg, 0.038 mmol) was dissolved in 1,4-dioxane (0.40 mL) and water (0.04 mL). 1N HCl (40 μL, 0.040 mmol) was added followed by 10% Pd/C (5 mg). A H₂ atmosphere was introduced via balloon. After 40 min of stirring at room temp. the reaction mixture was diluted with H₂O: 1,4-dioxane; 1:1 and filtered. The filtrate was lyophilized to afford 15 mg (100%) of 65 (Example 6 compound). IR (KBr): 1784 cm⁻¹

### Example 7

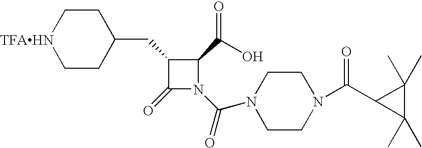

The title compound was prepared according to the following reaction scheme and as described below.

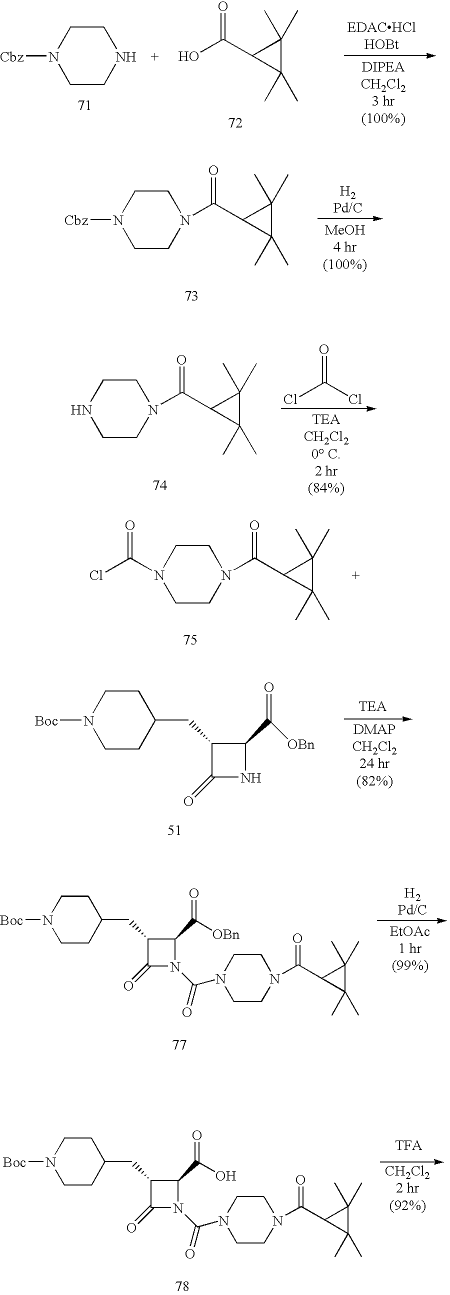

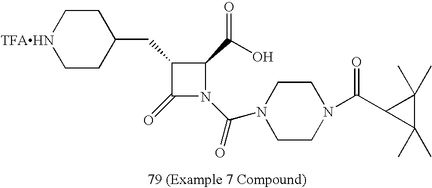

### Preparation of Example 7 Compound

1. Preparation of 73

EDAC.HCl (0.65 g, 3.37 mmol) was added to a stirred solution of compound 71 (0.62 g, 2.81 mmol), compound 72 (0.40 g, 2.81 mmol), HOBt (0.46 g, 3.37 mmol), and DIPEA (0.69 mL, 3.93 mmol) in CH₂Cl₂ (12 mL) at room temp. After 3 hr the reaction mixture was conc. and the residue was partitioned between EtOAc and 1N HCl. The organic phase was isolated, washed with 1N HCl, H₂O, sat. NaHCO₃, sat. NaCl, dried (MgSO₄), and conc. to give 0.97 g (100%) of compound 73.

2. Preparation of 74

Compound 73 (0.97 g, 2.82 mmol) was dissolved in MeOH (10 mL). 10% Pd/C (100 mg) was added and a H₂ atmosphere was introduced via balloon. After 4 hr the reaction mixture was filtered. The filtrate was conc. to afford 0.59 g (100%) of compound 74.

3. Preparation of 75

Phosgene (20% in toluene) (5.23 mL, 9.84 mmol) was added to a stirred solution of compound 74 (0.59 g, 2.81 mmol) and TEA (0.47 mL, 3.37 mmol) in CH₂Cl₂ (10 mL) at 0° C. After 2 hr the reaction mixture was conc. and the residue was triturated with Et₂O. The solution was filtered and the filtrate was conc. The crude product was purified by silica gel chromatography to give 0.64 g (84%) of compound 75.

4. Preparation of 77

Compound 75 (50 mg, 0.185 mmol) and compound 51 (prepared as described in Example 5) (62 mg, 0.154 mmol) were dissolved in CH₂Cl₂ (0.77 mL). TEA (30 uL, 0.216 mmol) was added followed by DMAP (4.0 mg, 0.031 mmol). After 24 hr the reaction mixture was conc. and the residue was partitioned between EtOAc and 1N HCl. The organic phase was isolated, washed with 1N HCl, dried (MgSO₄), and conc. The crude product was purified by silica gel chromatography to give 80 mg (82%) of compound 77.

5. Preparation of 78

Compound 77 (80 mg, 0.125 mmol) was dissolved in EtOAc (1 mL). 10% Pd/C (10 mg) was added and a H₂ atmosphere was introduced via balloon. After 1 hr the reaction mixture was diluted with EtOAc and filtered. The filtrate was conc. to afford 68 mg (99%) of compound 78.

6. Preparation of 79 (Example 7 Compound)

TFA (0.20 mL) was added dropwise to a stirred solution of compound 78 (68 mg, 0.124 mmol) in CH₂Cl₂ (0.60 mL) at 0° C. The reaction mixture was then stirred at room temp. After 2 hr the reaction mixture was conc. and placed under vacuum. The crude product was dissolved in H₂O and lyophilized. 64 mg (92%) of 79 (Example 7 compound) was obtained. LR-MS (ESI); (M+H) calc'd=449, found=449.

### Example 8

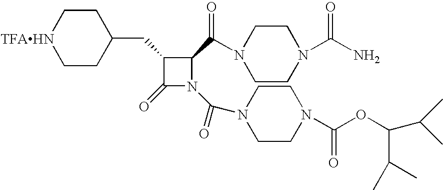

The title compound was prepared according to the following reaction sequence and as described below.

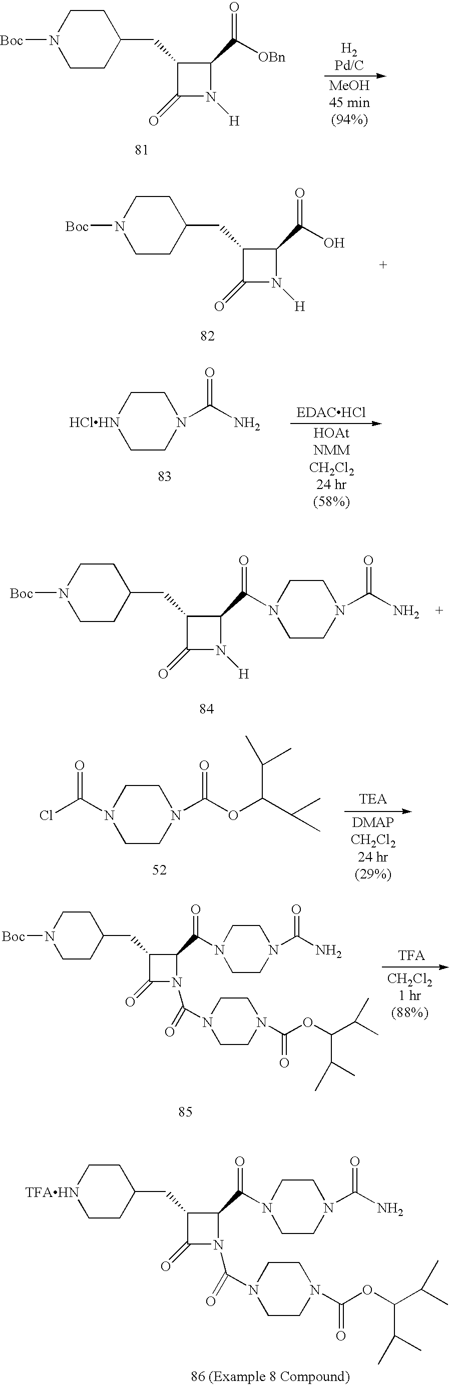

### Preparation of Example 8 Compound

51. Preparation of Compound 82

Example 5 compound 51 (210 mg, 0.522 mmol) was dissolved in MeOH (2.0 mL). 10% Pd/C (21 mg) was added and a H₂ atmosphere was introduced via balloon. After 45 min the reaction mixture was filtered. The filtrate was conc. to afford 154 mg (94%) of compound 82.

2. Preparation of Compound 83

This compound is prepared in Example 74 of U.S. Pat. No. 6,335,324.

3. Preparation of Compound 84

EDAC.HCl (44 mg, 0.229 mmol) was added to a stirred solution of compound 82 (55 mg, 0.176 mmol), compound 83 (35 mg, 0.211 mmol), HOAt (31 mg, 0.229 mmol), and NMM (48 μL, 0.440 mmol) in CH₂Cl₂ (0.70 mL) at room temp. After 24 hr the reaction mixture was conc. and the residue was partitioned between EtOAc and 5% KHSO₄. The organic phase was isolated, washed with sat. NaHCO₃, sat. NaCl, dried (MgSO₄), and conc. to give 43 mg (58%) of compound 84.

4. Preparation of Compound 85

Compound 84 (41 mg, 0.097 mmol) and Example 5 compound 52 (37 mg, 0.126 mmol) were dissolved in CH₂Cl₂ (0.50 mL). TEA (20 uL, 0.145 mmol) was added followed by DMAP (2.0 mg, 0.019 mmol). After 24 hr the reaction mixture was conc. and the residue was partitioned between EtOAc and 5% KHSO₄. The organic phase was isolated, washed with sat. NaCl, dried (MgSO₄), and conc. The crude product was purified by silica gel chromatography to afford 19 mg (29%) of compound 85.

5. Preparation of Compound 86 (Example 8 Compound)

TFA (0.125 mL) was added dropwise to a stirred solution of compound 86 (19 mg, 0.028 mmol) in CH₂Cl₂ (0.375 mL) at 0° C. The reaction mixture was then stirred at room temp. After 1 hr the reaction mixture was conc. and placed under vacuum. The crude product was dissolved in H₂O and lyophilized. 17 mg (88%) of 86 (Example 8 compound) was obtained. IR (KBr): 1784 cm⁻¹

### Example 9

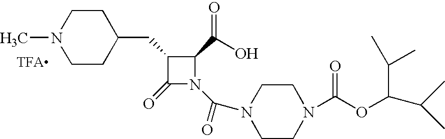

The title compound was prepared according to the following reaction scheme and as described below.

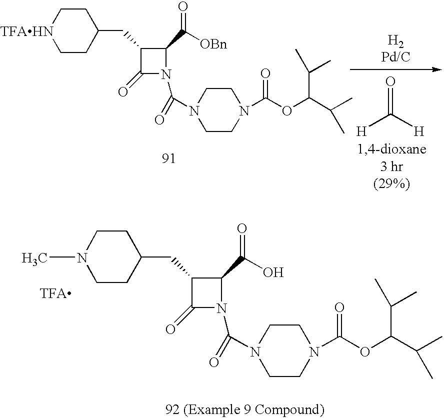

### Preparation of Example 9 Compound

1. Preparation of Compound 91

See Example 11 Part 1 for preparation.

2. Preparation of Compound 92 (Example 9 Compound)

Compound 91 (86 mg, 0.128 mmol) was dissolved in 1,4-dioxane (0.8 mL). Formaldehyde (37 wt % in H₂O) (44 uL) was added followed by 10% Pd/C (15 mg) A H₂ atmosphere was introduced via balloon. After 3 hr the reaction mixture was diluted with H₂O: 1,4-dioxane; 1:1, filtered, and the filtrate was lyophilized. The crude product was purified by PREP HPLC to afford 22 mg (29%) of compound 92 (Example 9 compound). IR (KBr): 1787 cm⁻¹

### Example 10

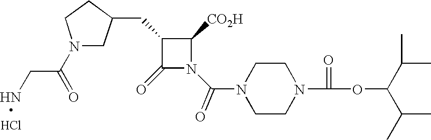

The title compound was prepared according to the following reaction sequence and as described below.

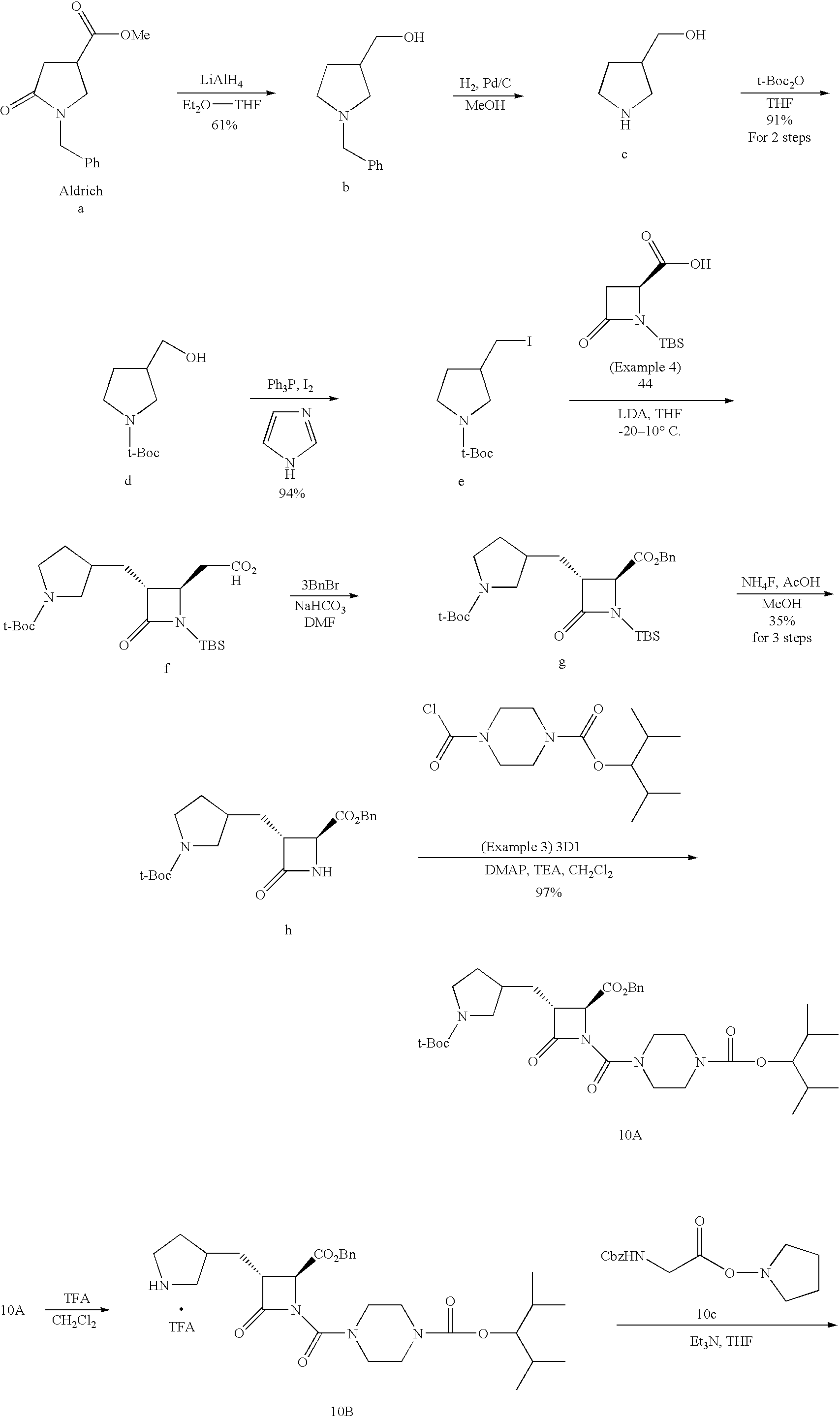

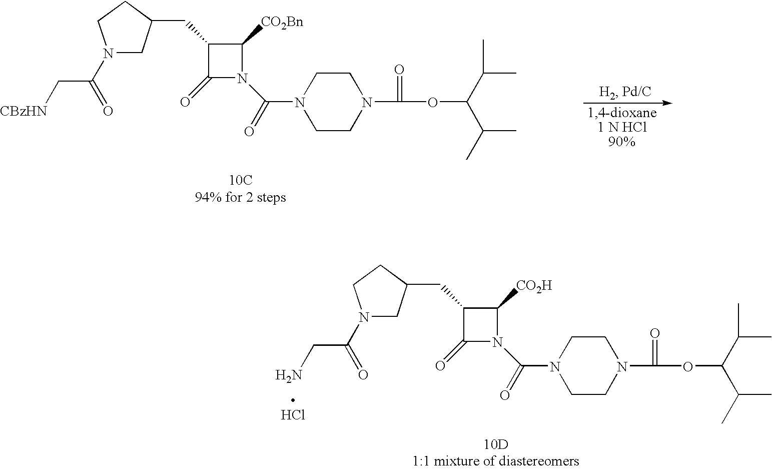

### Preparation of Example 10 Compound

1. Preparation of Compound 10A

A. Preparation of Compound b

Compound b was prepared by a similar procedure to Wu's (J. Org. Chem. 1961, 1519.) except using THF to dissolve methyl l-benzyl-5-oxo-3pyrrolidinecarboxylate:

A solution of methyl 1-benzyl-5-oxo-3pyrrolidinecarboxylate (a) (8.00 g, 34.30 mmol) in 10 mL of anhydrous THF was slowly added to a slurry of powdered lithium aluminum hydride (1.82 g, 48.01 mmol) in 30 mL of absolute ether. The addition was made over a period of 0.5 hour with efficient stirring so as to maintain a moderate reflux rate. When the addition was complete, refluxing and stirring was continued for 2 hours, after which the reaction mixture was left at room temperature. The mixture was quenched with 3 mL of water, and stirred for 2 hours. The white precipitate was filtered and washed with 2×30 mL of ether. The solid was extracted with Soxhlet type apparatus in EtOH for 8 hours. Th EtOH was removed. The residue was washed with Et₂O (3×20 mL). The Et₂O solutions were combined, dried over MgSO₄, and concentrated. The residue was distilled under vacuum. The fraction at 137-145° C./1 mmHg was collected to give compound b (4.0 g) as a colorless oil.

B. Preparation of Compound c and Compound d

To a solution of the amino alcohol b (1.50 g, 7.84 mmol) in 100 mL of MeOH was added Pd/C (400 mg). The mixture was stirred for 8 hours under H₂ atomphere. MeOH was removed to give residue c as colorless oil. The residue c was dissolved in 50 mL of THF. t-Boc₂O (2.50 g, 11.76 mmol) was added The solution was stirred for 8 hours and THF was removed. The residue was partitioned between ether (200 mL) and 0.25 M KHSO4 (100 mL). The ether layer was washed with sat. NaHCO₃ (20 mL) and brine, dried over MgSO₄, and concentrated. The residue was purified with silica gel chromatography (CH₂Cl₂: MeOH=10:1, Rf=0.35) to provide d (1.40 g) as a colorless oil. MS 202.4 (M+H)⁺.

C. Preparation of Compound e

Triphenyl phosphine (2.89 g, 11.00 mmol) was dissolved in 25 mL of CH₂Cl₂. Imidazole (0.75 g, 11.00 mmol) was added followed by iodine (2.79 g, 11.00 mmol). The solution of compound d in 5 mL of CH₂Cl₂ was slowly added. The reaction mixture was stirred for at room temperature for 2 hours, and then filtered. The filtrate was concentrated. The residue was triturated with EtOAc, The EtOAc solution was washed with 5% NaS₂O₃ and brine, dried over MgSO₄, and concentrated. The residue was purified by silica gel chromatography (ethyl acetate:hexane=6:1, Rf=0.25) to afford compound e (2.30 g) as a colorless oil. MS: 312.1 (M+H)⁺.

D. Preparation of Compound f

To a solution of diisopropylamine (0.68 mL, 4.82 mmol) in 5 mL of THF was added BuLi (1.6 M, 2.88 mL, 4.62 mmol) at −15° C. The solution was cooled to −78° C. and stirred for 20 minutes. A solution of β-lactam 44 (from Example 4) (500 mg, 2.20 mmol) in 2 mL of THF was added dropwise. The solution was warmed to −20° C. to −10° C. and stirred at this temperature for 30 minutes. Then a solution of compound e (1.03 g, 3.30 mmol) in 3 mL of THF was added. The reaction mixture was stirred for 8 hours and then quenched with 5% KHSO4 until PH=3-4. The aqueous solution was extracted with EtOAc (3×20 mL). Organic extracts were combined, washed with brine, and concentrated. The residue was taken up with 60 mL of ether and the solution was extracted with sat. NaHCO₃ solution (3×20 mL). The basic aqueous layers were washed with ether (20 mL), cooled to 0° C., acidified with 10% KHSO4 until PH=3-4, extracted with EtOAc (3×20 mL). The extracts were washed with brine, dried over MgSO₄, and concentrated to provide compound f as a colorless oil (0.60 g). MS 413.3 (M+H)⁺.

E. Preparation of Compound g

Compound f (0.60 g 1.45 mmol) was dissolved in 10 mL of DMF. NaHCO₃ (244 mg, 2.90 mmol) followed by benzyl bromide. The reaction mixture was stirred overnight at room temperature, then partitioned between water (20 mL) and EtOAc (60 mL). The organic layer was washed with brine, dried over MgSO4, and concentrated to give crude compound g as colorless oil.

F. Preparation of Compound h

The crude compound g was dissolved in 10 mL of methanol. Acetic acid (0.25 mL, 4.35 mmol) and a solution of NH₄F (0.5 M, 2.90 mL, 1.45 mmol) in methanol was added. The solution was stirred for 1 hour. The solvents were replaced with 60 mL of CH₂Cl₂, washed with sat. NaHCO₃ solution (10 mL) and brine (10 mL), dried over MgSO₄, and concentrated. The residue was purified with silica gel chromatography (ethyl acetate:hexane=1:1, Rf=0.12) to afford compound h (300 mg) as a white solid.

G. Preparation of Compound 10A

To a solution of ester h (260 mg, 0.67 mmol) in 5 mL of CH₂Cl₂ was added triethyl amine (0.11 mL, 0.81 mmol), acid chloride (compound 3D1 in Example 3) (233 mg, 0.81 mmol), and DMAP (4.0 mg, 0.034 mmol). The solution was stirred for 3 hour. The solvent was removed. The residue was purified with silica gel chromatography (ethyl acetate:hexane=2:1, Rf=0.26) to afford compound 10A (417 mg) as a colorless oil.

2. Preparation of Compound 10B

Compound 10A (85 mg, 0.13 mmol) was dissolved in 0.8 mL of CH₂Cl₂. The solution was cooled to 0° C. and TFA (0.2 mL) was added dropwise. The ice-bath was removed The mixture was stirred at room temperature for 1 hour. The solvents were removed under vacuum to generate crude compound 10B as colorless oil.

3. Preparation of Compound 10C

Compound 10B (0.13 mmol) was dissolved in 1 mL of THF. Cbz-Gly-OSu (10 c) (50 mg, 0.16 mmol) was added followed by triethyl amine. The reaction mixture was stirred overnight at room temperature, diluted with ethyl acetate (50 mL), washed with 5% KHSO4, Sat. NaHCO₃ solution, and brine, dried with MgSO₄ and concentrated. The residue was purified with silica gel chromatography (ethyl acetate:hexane=2:3 to 1:2) to afford compound 10C (90 mg) as a colorless oil.

4. Preparation of Compound 10D

A mixture of compound 10C (68 mg, 0.092 mmol), Pd/C (10%, 15 mg) and 1 N HCl (92 μL, 0.092 mmol) in 1,4-dioxane (1 mL) was stirred under hydrogen atmosphere (hydrogen balloon) at room temperature for 8 hours. Analytical HPLC indicated the completion of the reaction. The reaction mixture was diluted 2 mL of water, filtered through a celite pad, and lyophilized to give title compound 10D (Example 10 compound) (45 mg) as a white powder. Purity by anal HPLC: 98%; MS: 510.3 (M+H)⁺ , 508.2 (M−H)⁻; IR: (KBr pellet) u 1787 cm⁻¹.

### Example 11

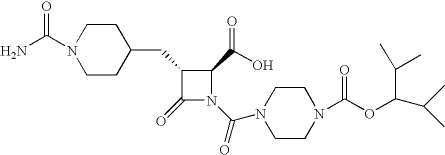

The title compound was prepared according to the following reaction sequence and as described below.

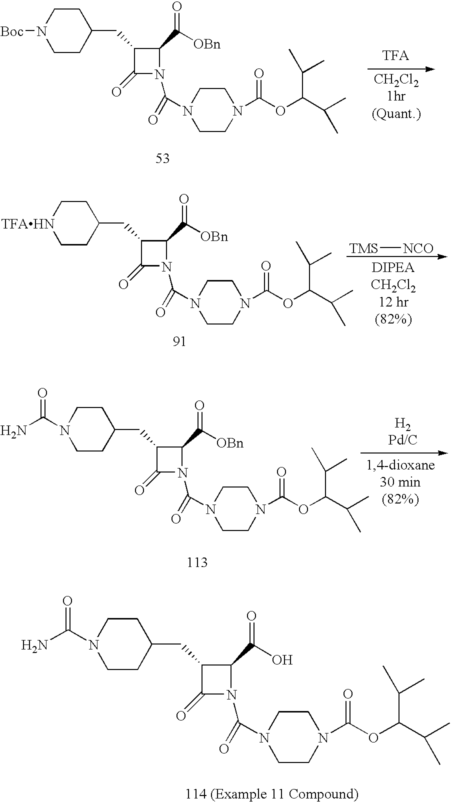

### Preparation of Example 11 Compound

1. Preparation of Compound 91

TFA (0.70 mL) was added dropwise to a stirred solution of compound 53 (prepared in Example 5) (172 mg, 0.262 mmol) in CH₂Cl₂ (2.1 mL) at 0° C. The reaction mixture was then stirred at room temp. After 1 hr the reaction mixture was conc. and placed under vacuum. 176 mg (100%) of compound 112 was obtained.

2. Preparation of Compound 113

TMS-NCO (25 uL, 0.188 mmol) was added to a stirred solution of compound 91 (67 mg, 0.100 mmol) and DIPEA (44 uL, 0.250 mmol) in CH₂Cl₂ (1 mL) at 0° C. The reaction mixture was then stirred at room temp. After 12 hr the reaction mixture was partitioned between CH₂Cl₂ and 5% KHSO₄. The organic phase was isolated, dried (MgSO₄), and conc. The crude product was purified by silica gel chromatography to give 49 mg (82%) of compound 113.

3. Preparation of Compound 114

Compound 113 (49 mg, 0.082 mmol) was dissolved in 1,4-dioxane (0.5 mL). 10% Pd/C (10 mg) was added and a H₂ atmosphere was introduced via balloon. After 30 min of stirring at room temp. the reaction mixture was diluted with 1,4-dioxane and filtered. The filtrate was lyophilized to afford 34 mg (82%) of compound 114 (Example 11 compound). IR (KBr): 1786 cm⁻¹

### Example 12

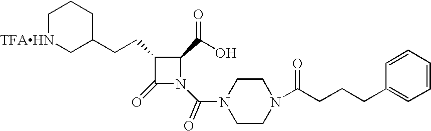

The title compound was prepared according to the following reaction sequence and as described below.

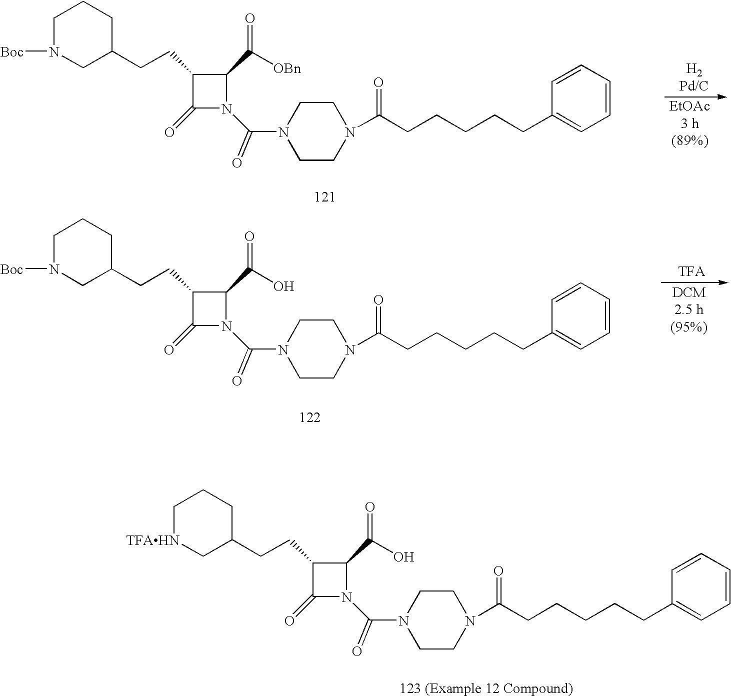

### Preparation of Example 12 Compound

1. Preparation of Compound 121

This compound is prepared as described in U.S. Pat. No. 6,335,324, Example 202 Part h.

2. Preparation of Compound 122

Compound 121 (84 mg, 0.120 mmol) was dissolved in EtOAc (1.50 mL). 10% Pd/C (12 mg) was added and a H₂ atmosphere was introduced via balloon. After 3 hr the reaction mixture was diluted with EtOAc and filtered. The filtrate was conc. to afford 65 mg (89%) of compound 122.

3. Preparation of Compound 123 (Example 12 Compound)

TFA (0.25 mL) was added dropwise to a stirred solution of compound 122 (65 mg, 0.106 mmol) in CH₂Cl₂ (0.75 mL) at 0° C. The reaction mixture was then stirred at room temp. After 2.5 hr the reaction mixture was conc. and placed under vacuum. The crude product was dissolved in H₂O (1.0 mL) and 1,4-dioxane (3.0 mL) and lyophilized. 63 mg (95%) of compound 123 (Example 12 compound) was obtained. LR-MS (ESI); (M+H) calc'd=513, found=513.

Following the procedures set out in Examples 1 to 12 and in the reaction schemes, the following compounds were prepared.

### Examples 13 to 17

| Example No. | Structure |
|---|---|
| 13 | 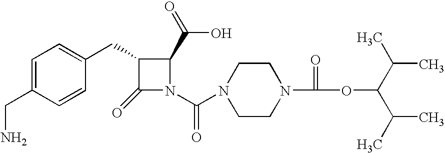 |
| 14 | 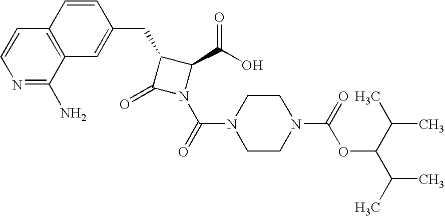 |
| 15 | 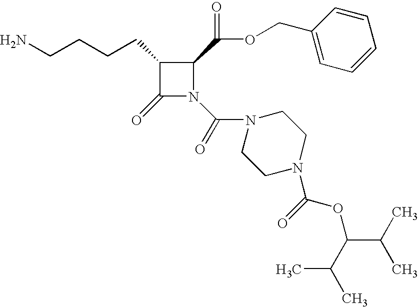 |
| 16 | 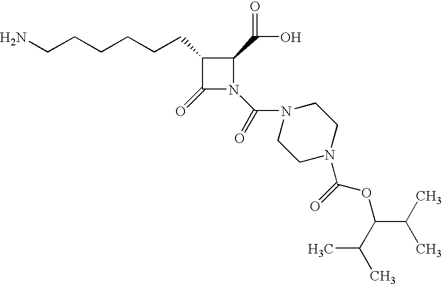 |
| 17 | 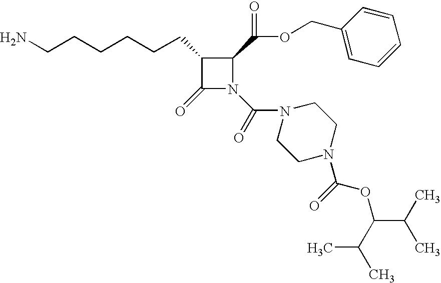 |

### Example 18

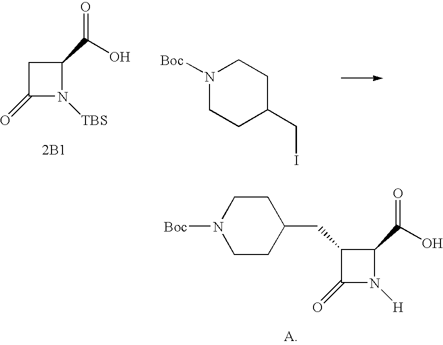

A. To a solution of iPr₂NH (6.42 ml, 45.8 mmol) in THF (15 ml) at 0° C. was added nBuLi (1.6 M, 28 ml, 45 mmol). After stirring at room temperature for 1 hour, the reaction mixture was cooled to −78° C. and treated dropwise over 5 minutes with a solution of 2B1 (5 g, 21.8 mmol in 15 ml THF). The reaction mixture was then warmed to −20° C. and a slurry formed. THF (15 ml) was added to loosen the slurry and the slurry was stirred vigorously for 2.5 hour at −20° C. A solution of N-Boc-4-(iodomethyl)piperidine (10.63 g, 32.7 mmol in 15 ml THF) was added dropwise over 3 minutes and the resulting solution was stirred at −20° C. for 18 hours. The reaction mixture was partitioned between EtOAc (200 ml) and water (200 ml). After mixing and separation, the organic phase was extracted once more with water (150 ml). The combined water layers were then washed with EtOAc (2×200 ml). The aqueous phase was diluted with EtOAc (200 ml) and with rapid stirring brought to pH 2-3 with portionwise addition of solid KHSO₄. The EtOAc layer was removed and the aqueous layer was extracted with EtOAc (3×200 ml). The EtOAc extracts from the acidified aqueous layer were combined, dried over Na₂SO₄, filtered and concentrated.

The residue was taken up in THF (30 ml), treated with TBAF (1.0 M THF, 22 ml) and stirred at room temperature for 2 hours. The reaction mixture was partitioned between EtOAc and 0.5N KHSO4. The aqueous phase was then extracted with EtOAc (4×). The combined EtOAc extracts were dried over MgSO4, filtered and concentrated to give Part A compound (2.8 g, 42%).

1H-NMR (400 MHz, CDCl3) δ 8-9 (bs, 1H), 6.82 (s, 1H), 4.1 (m, 2H), 3.88 (d, 1H, j 2.47), 3.36 (m, 1H), 2.67 (m, 2H), 1.6-1.9 (m, 6H), 1.43 (s, 9H), 1.1 (m, 1H).

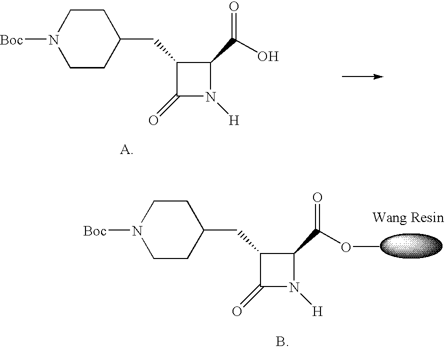

B. To a suspension of Part A compound (4.1 g, 13.14 mmol) in DCM:THF (1:1, 100 ml) at room temperature was added MSNT (3.89 g, 13.14 mmol) followed by N-methylimidazole (2.7 ml). The reaction mixture was stirred for 3 to 5 minutes until all materials dissolved. The reaction mixture was then added to DCM rinsed Wang Resin (12 g dry weight, 0.7 mmol/g theoretical load capacity) and the reaction was aggitated for 48 hours at RT. The resin was washed with THF (100 ml) and the eluent was collected and saved. The resin was then washed with DMF (4×100 ml), THF (4×100 ml), DCM (4×100 ml) and Et₂O (4×100 ml). The resin was dried under vacuum for 24 hours to give resin bound Part B compound (14.8 g).

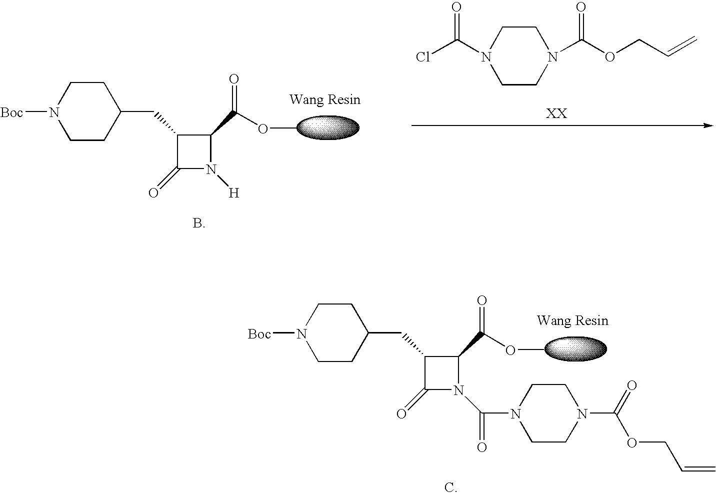

C. To a suspension of Part B compound resin (14.8 g, ˜8.4 mmol load) in DCM (65 ml) was added carbamoyl chloride XX (3.9 g, 16.8 mmol), Et₃N (11 ml) and DMAP (3.8 g). The reaction was aggitated at room temperature for 24 hours. The resin was drained and then washed with DMF (4×75 ml), THF (4×75 ml), DCM (4×75 ml) and Et₂O (4×75 ml). The resin was then dried under vacuum overnight to give Part C compound resin (15.25 g, 96% purity determined by HPLC of a 15 mg sample treated with 20% TFA/DCM for 1.5 hours. HPLC conditions—Phenomenex-Prime S5 C18 4.6×50 mm, 4 min gradient, 0 to 100% B solvent, 1 minute hold, 4 ml/min, A=10% MeOH/Water+0.2% H₃PO₄, B=90% MeOH/Water+0.2% H₃PO₄. Retention time 2.15 minutes).

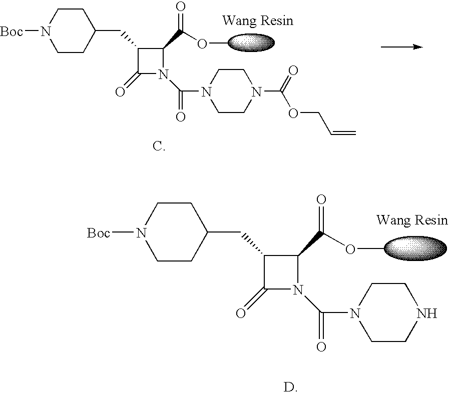

D. To a suspension of Part C compound resin (15.25 g, ˜8.4 mmol load) in DCM (75 ml) at room temperature was added PhSiH₃ (15.5 ml, 126 mmol) followed by Pd(PPh₃)₄ (485 mg, 0.42 mmol). The reaction was aggitated for 6 hours at room temperature. The resin was then washed with DMF (4×100 ml), 30% MeOH/DCM (4×100 ml), DCM (4×100 ml) and Et₂O (3×100 ml). The resin was vacuum dried overnight to give Part D compound resin (14.2 g, 95% purity determined by HPLC of a 42 mg sample treated with 20% TFA/DCM for 1.5 hours which gave 0.29 mmole of product. HPLC conditions—Phenomenex-Prime S5 C18 4.6×50 mm, 4 min gradient, 0 to 100% B solvent, 1 minute hold, 4 ml/min, A=10% MeOH/Water+0.2% H₃PO₄, B=90% MeOH/Water+0.2% H₃PO₄. Retention time 0.243 minutes).

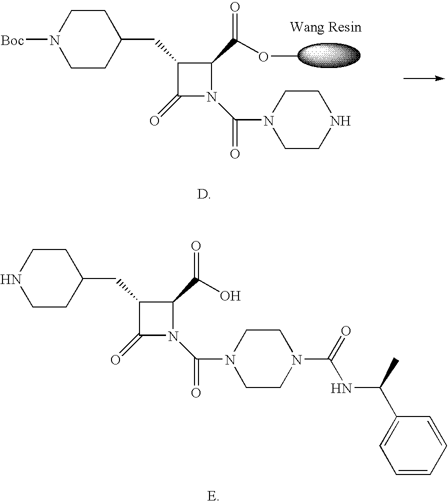

E. To S-(-)-α-methylbenzylisocycante (0.072 mmol) was added 1 ml of a solution comprised of DCM (30 ml), Et3N (401 μl) and DMAP (87 mg). After aggitating to dissolve all materials, Part D compound resin (35 mg) was added and the reaction was aggitated for 36 hours. The resin was washed with DMF:DMSO (1:1, 4×5 ml) and DCM (4×5 ml). The resin was then treated with 20% TFA/DCM (1.2 ml) for 2 hours. Eluent was collected and solvent reduced to provide product E (12.2 mg); 87% purity by LCMS, (M+H)+=472.36.

### Example 19

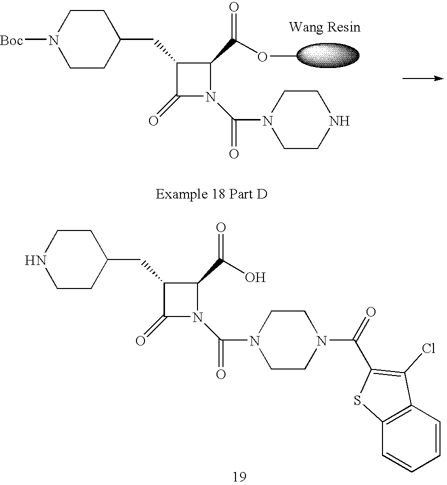

To 3-chlorobenzo[B]thiophene-2-carboxylic acid (0.072 mmol) was added a solution of HOAT in DMA (0.5 ml, 0.12 mmol HOAT in 0.5 ml DMA). After sonicating to dissolve all materials, a solution of DIC in DCM (0.5 ml, 0.12 mmol DIC in 0.5 ml DCM). After agitating the reaction mixture for 1 minute, the reaction mixture was then transferred to a reacting vessel containing Part D compound resin (35 mg). The reaction was aggitated for 36 hours at room temperature. The resin was washed with DMF:DMSO (1:1, 4×5 ml) and DCM (4×5 ml). The resin was then treated with 20% TFA/DCM (1.2 ml) for 2 hours. Eluent was collected and solvent reduced to provide product (14.1 mg); 90% purity by LCMS, (M+H)+=519.22.

Following the procedures set out in Examples 18 and 19 and in the reaction schemes, the following compounds were prepared:

The following additional compounds were prepared following the procedures set out in the Examples and reaction schemes.

### Examples 20 to 131

| Example | | | Molecular | (M + H)+ |
|---|---|---|---|---|
| No. | Structre | | Weight | observed |
| 20 | 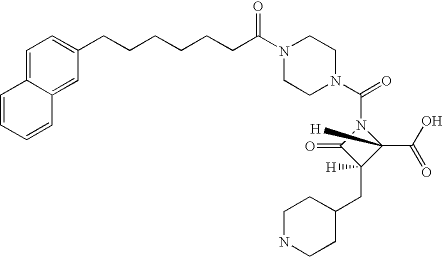 | | 562.72 | 563.43 |
| 21 | 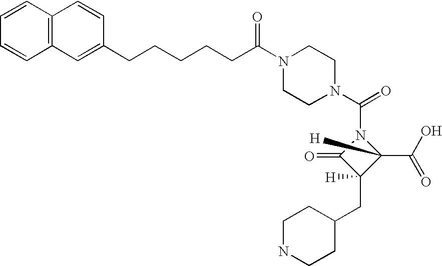 | | 548.69 | 549.40 |
| 22 | 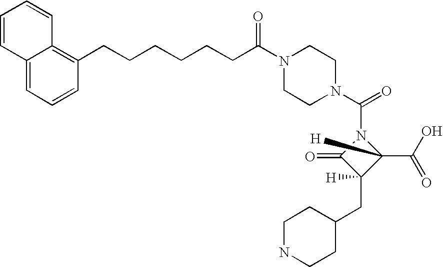 | | 562.72 | 563.43 |
| 23 | 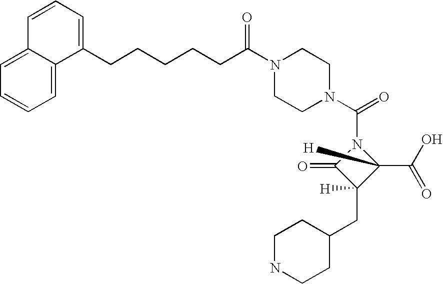 | | 548.69 | 549.39 |
| 24 | 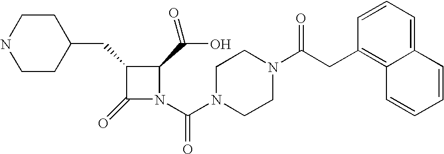 | | 492.58 | 493.32 |
| 25 | 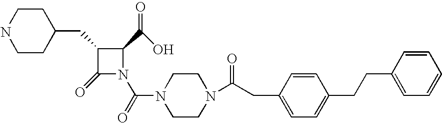 | | 546.67 | 547.43 |
| 26 | 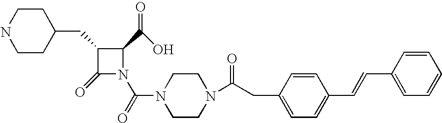 | | 544.86 | 545.29 |
| 27 | 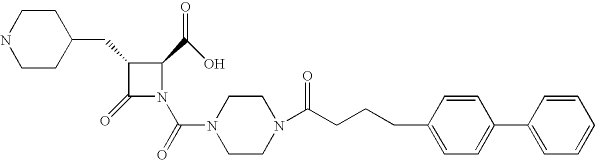 | | 546.67 | 547.42 |
| 28 | 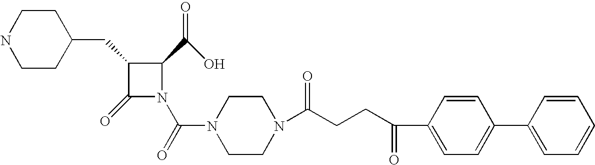 | | 560.66 | 561.44 |
| 28A | 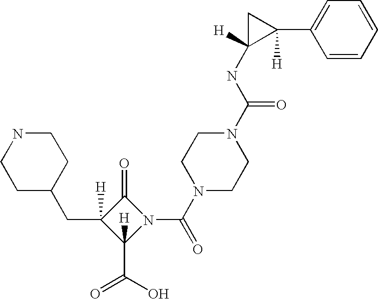 | Chiral | 483.566 | 484.3 |
| 29 | 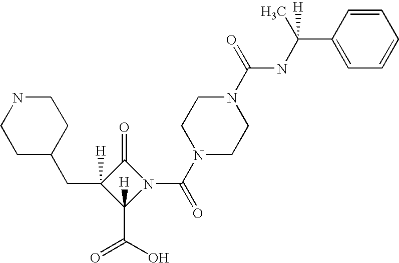 | Chiral | 471.555 | 472.36 |
| 30 | 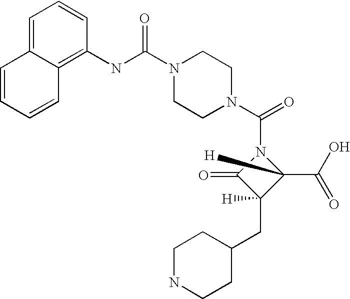 | Chiral | 493.561 | 494.34 |
| 31 | 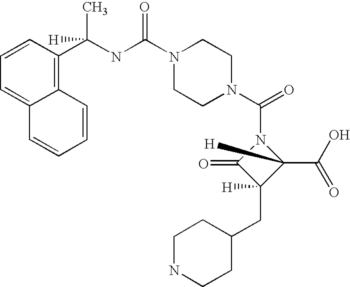 | Chiral | 521.615 | 522.37 |
| 32 | 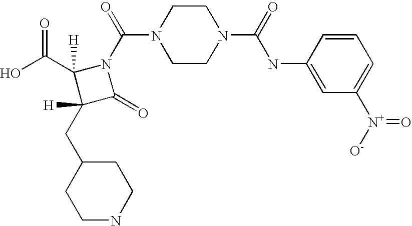 | Chiral | 488.498 | 489.35 |
| 33 | 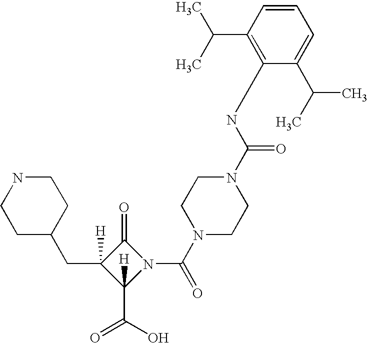 | Chiral | 527.662 | 528.4 |
| 34 | 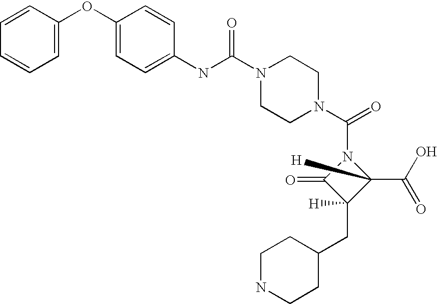 | Chiral | 535.598 | 536.32 |
| 35 | 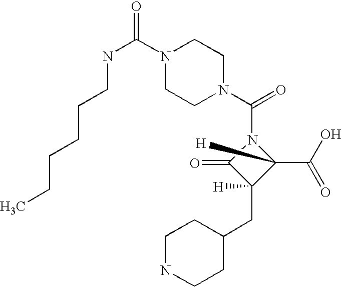 | Chiral | 451.564 | 452.38 |
| 36 | 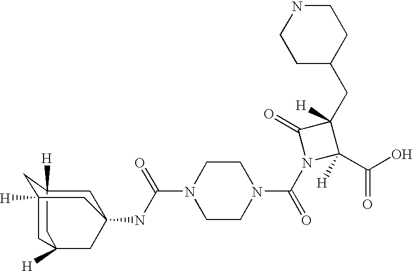 | Chiral | 501.624 | 502.37 |
| 37 | 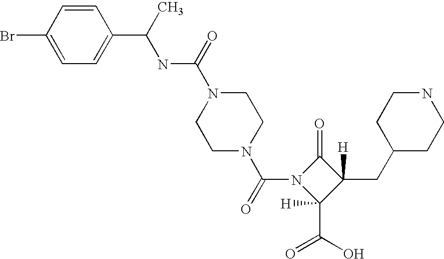 | Chiral | 550.451 | 550.25, 552.24 |
| 38 | 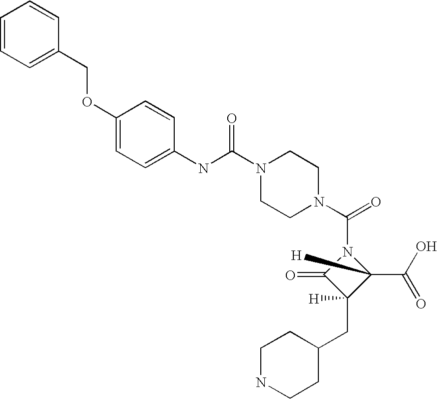 | Chiral | 549.625 | 550.37 |
| 39 | 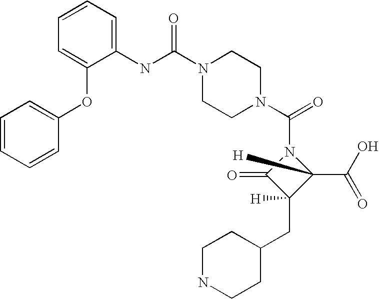 | Chiral | 535.598 | 536.33 |
| 40 | 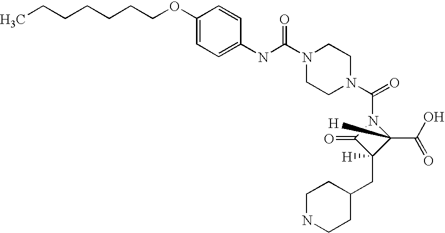 | Chiral | 557.688 | 558.42 |
| 41 | 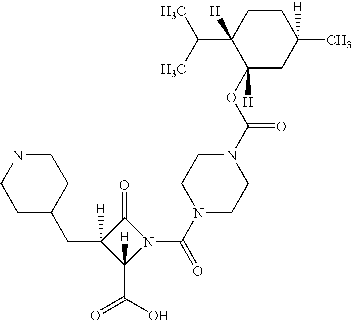 | Chiral | 506.64 | 507.4 |
| 42 | 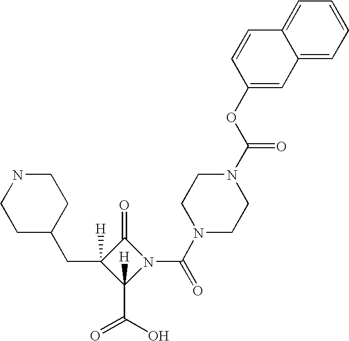 | Chiral | 494.545 | 495.26 |
| 43 | 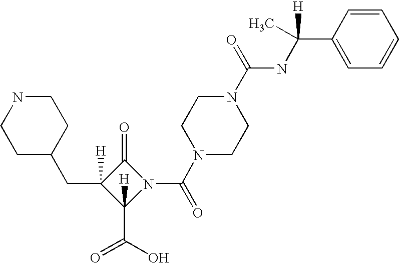 | Chiral | 471.555 | 472.36 |
| 44 | 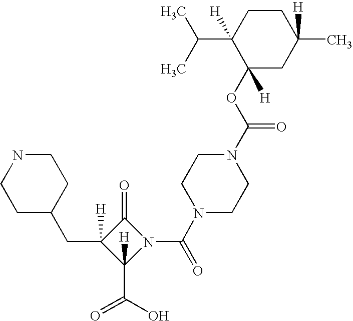 | Chiral | 506.64 | 507.35 |
| 45 | 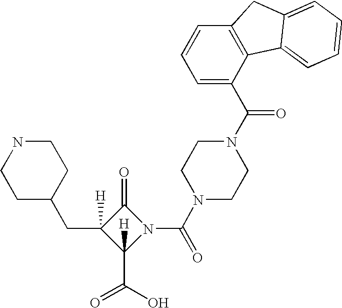 | Chiral | 516.595 | 517.26 |
| 46 | 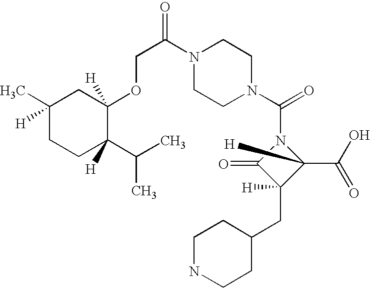 | Chiral | 520.667 | 521.35 |
| 47 | 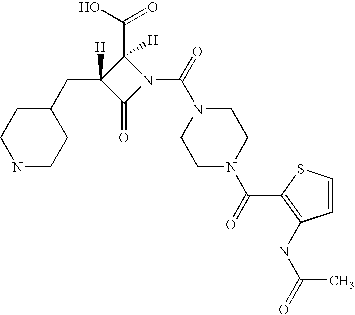 | Chiral | 491.566 | 492.25 |
| 48 | 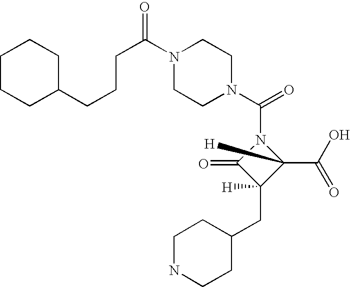 | Chiral | 476.614 | 477.37 |
| 49 | 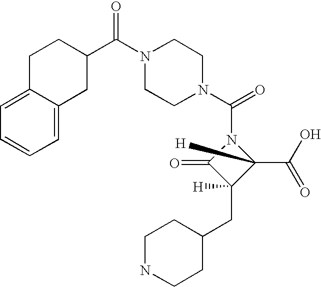 | Chiral | 482.578 | 483.32 |
| 50 | 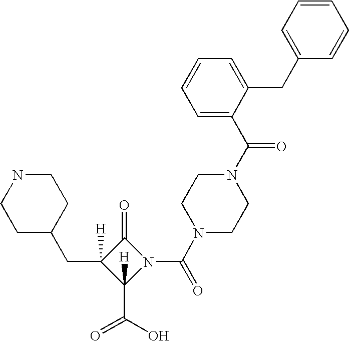 | Chiral | 518.611 | 519.27 |
| 51 | 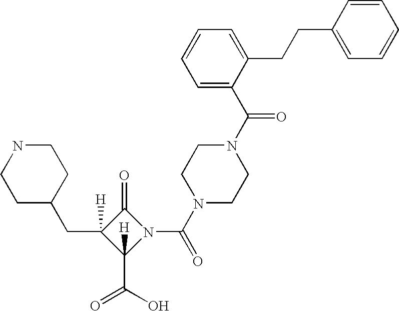 | Chiral | 532.637 | 533.31 |
| 52 | 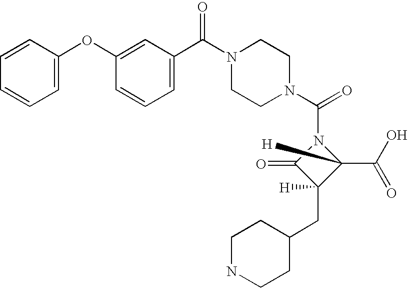 | Chiral | 520.583 | 521.25 |
| 53 | 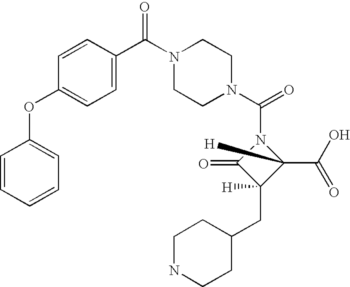 | Chiral | 520.583 | 521.25 |
| 54 | 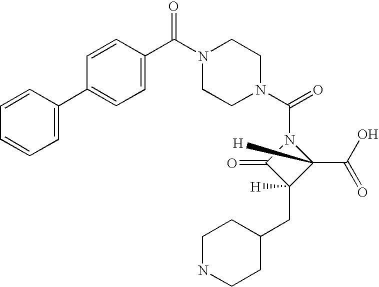 | Chiral | 504.584 | 505.27 |
| 55 | 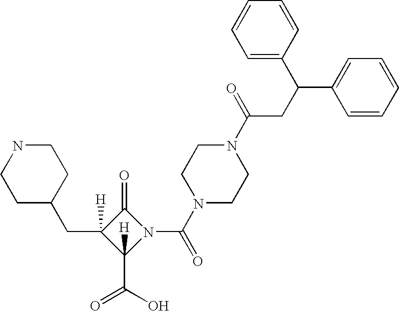 | Chiral | 532.637 | 533.31 |
| 56 | 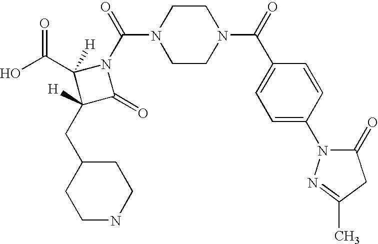 | Chiral | 524.575 | 525.32 |
| 57 | 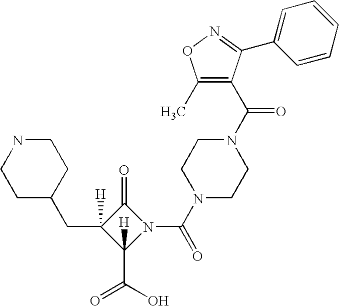 | Chiral | 509.56 | 510.25 |
| 58 | 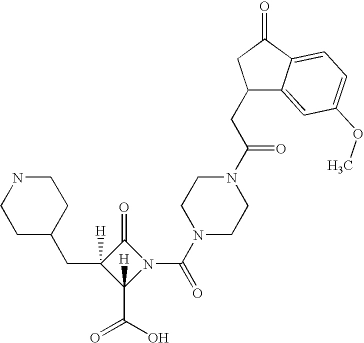 | Chiral | 526.587 | 527.27 |
| 59 | 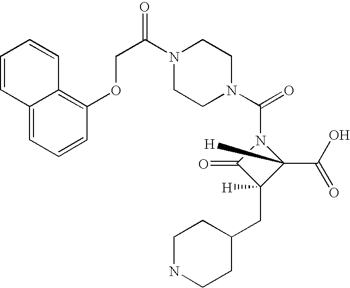 | Chiral | 580.572 | 509.24 |
| 60 | 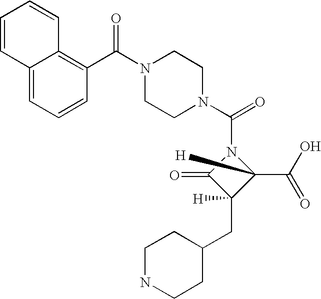 | Chiral | 478.546 | 479.29 |
| 61 | 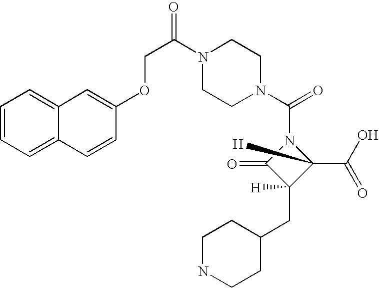 | Chiral | 508.572 | 509.25 |
| 62 | 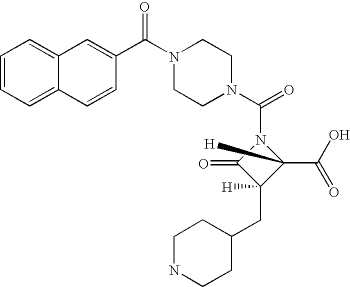 | Chiral | 478.546 | 479.31 |
| 63 | 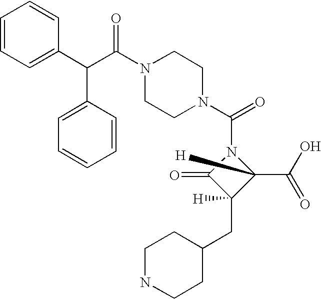 | Chiral | 518.611 | 519.27 |
| 64 | 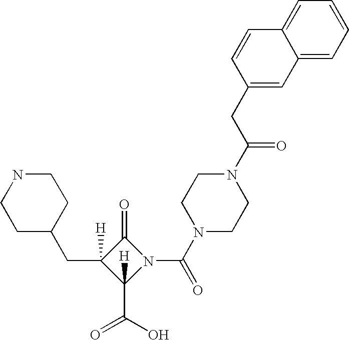 | Chiral | 492.573 | 493.28 |
| 65 | 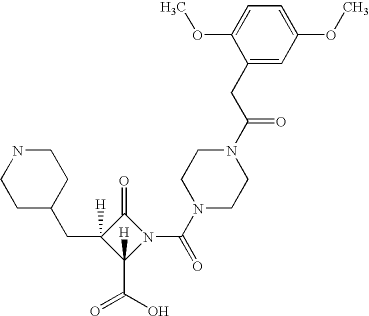 | Chiral | 502.565 | 503.28 |
| 66 | 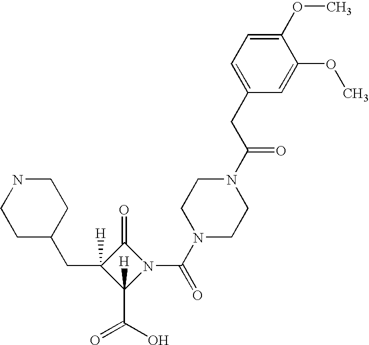 | Chiral | 502.565 | 50.328 |
| 67 | 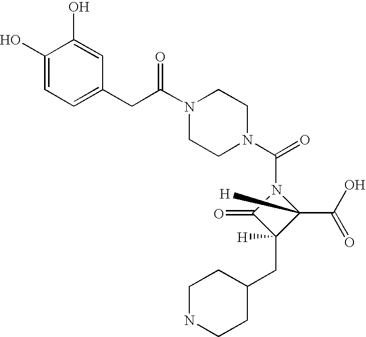 | Chiral | 474.511 | 475.3 |
| 68 | 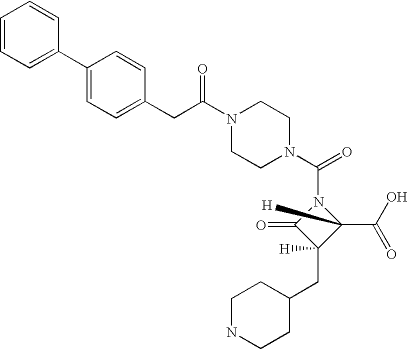 | Chiral | 518.611 | 519.27 |
| 69 | 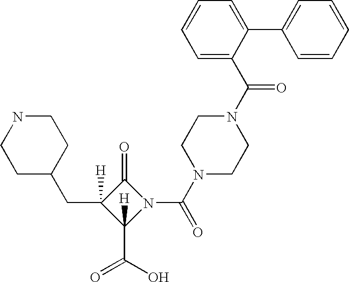 | Chiral | 504.584 | 505.27 |
| 70 | 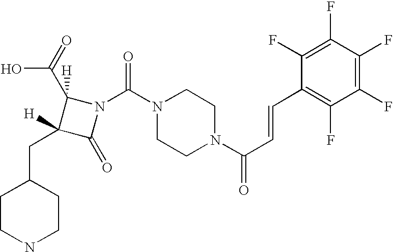 | Chiral | 544.474 | 545.25 |
| 71 | 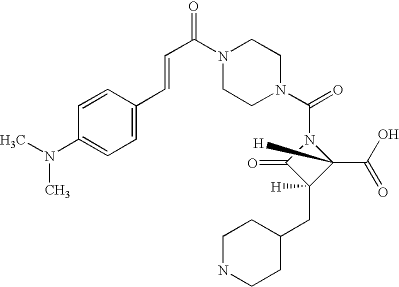 | Chiral | 497.592 | 498.33 |
| 72 | 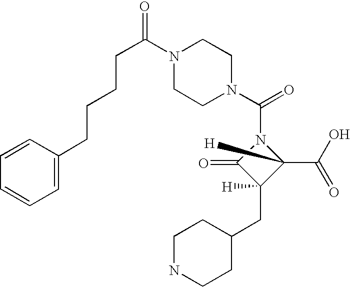 | Chiral | 484.593 | 485.33 |
| 73 | 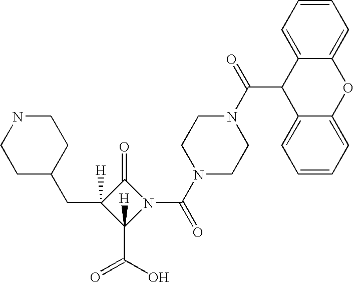 | Chiral | 532.594 | 533.27 |
| 74 | 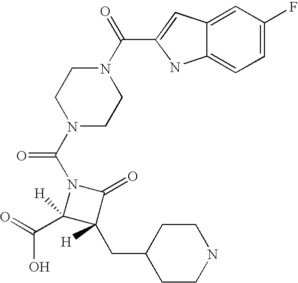 | Chiral | 485.513 | 486.29 |
| 75 | 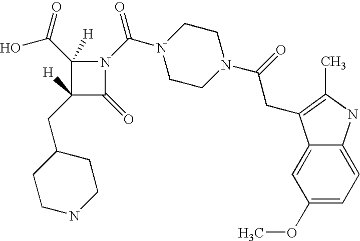 | Chiral | 525.602 | 526.33 |
| 76 | 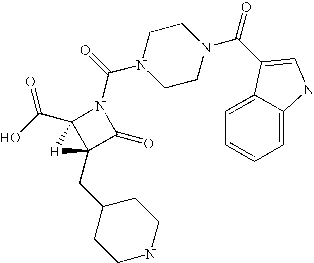 | Chiral | 467.523 | 468.3 |
| 77 | 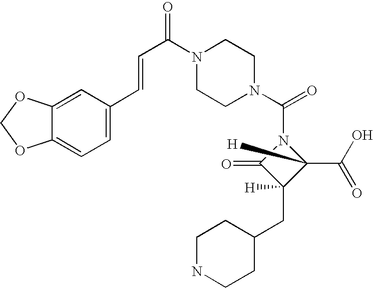 | Chiral | 498.533 | 499.25 |
| 78 | 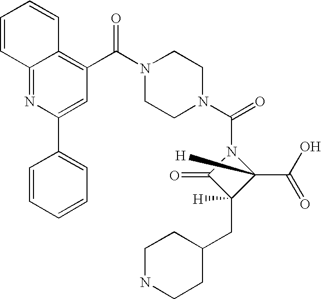 | Chiral | 555.632 | 556.33 |
| 79 | 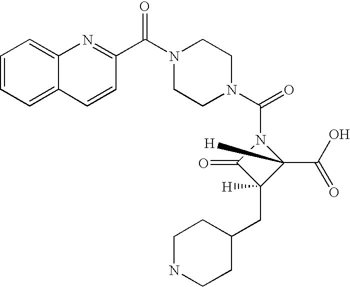 | Chiral | 479.534 | 480.29 |
| 80 | 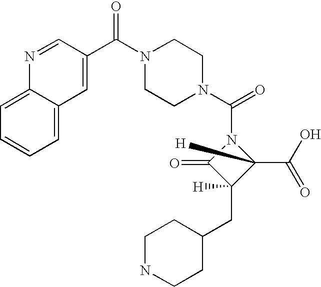 | Chiral | 479.534 | 480.31 |
| 81 | 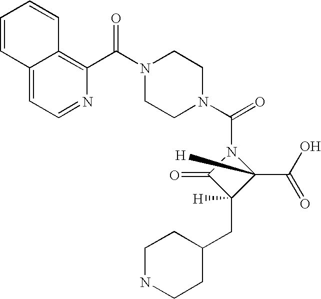 | Chiral | 479.534 | 480.28 |
| 82 | 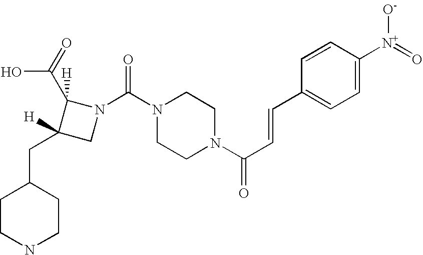 | Chiral | 499.521 | 500.3 |
| 83 | 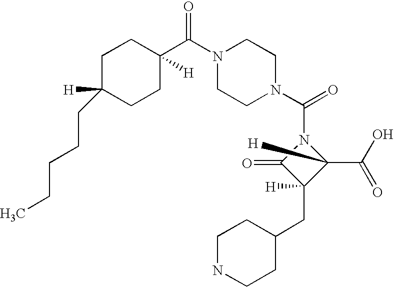 | Chiral | 504.668 | 505.37 |
| 84 | 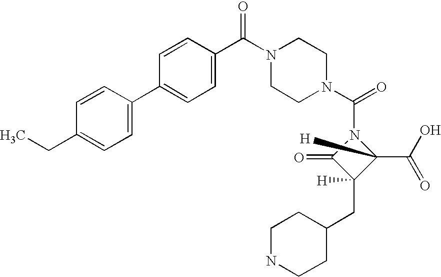 | Chiral | 532.637 | 533.31 |
| 85 | 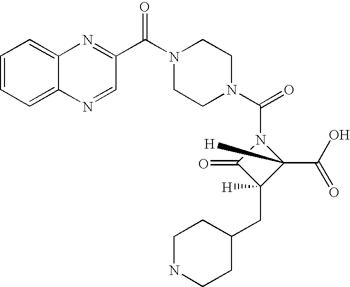 | Chiral | 480.522 | 481.27 |
| 86 | 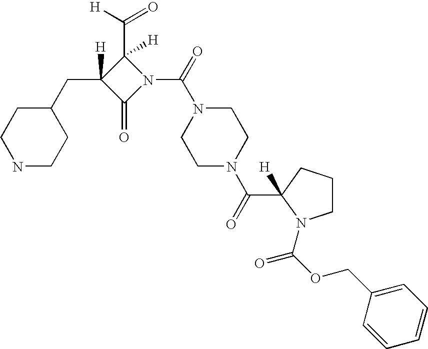 | Chiral | 555.628 | 556.34 |
| 87 | 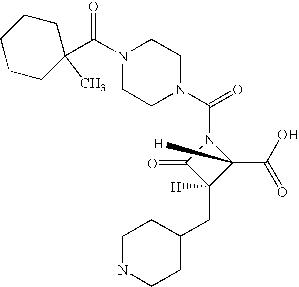 | Chiral | 448.56 | 449.33 |
| 88 | 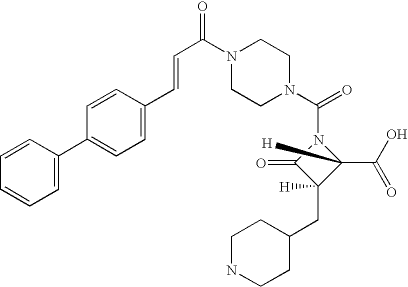 | Chiral | 530.622 | 531.33 |
| 89 | 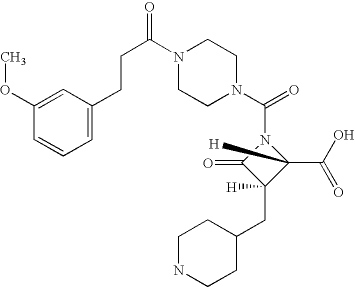 | Chiral | 486.566 | 487.33 |
| 90 | 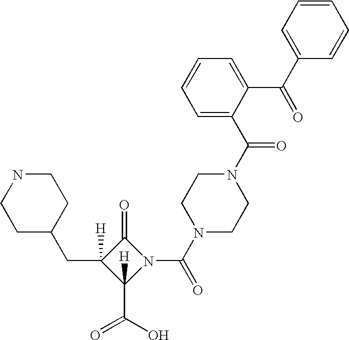 | Chiral | 532.594 | 533.32 |
| 91 | 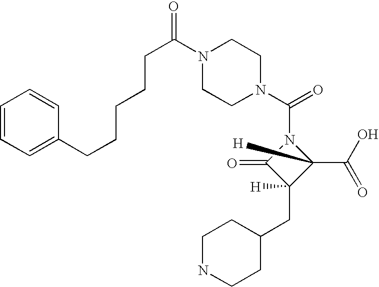 | Chiral | 498.62 | 499.38 |
| 92 | 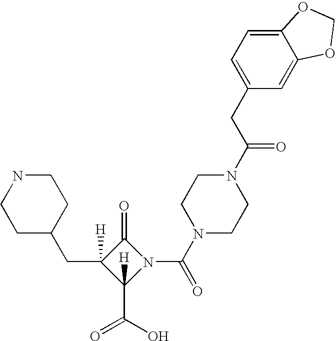 | Chiral | 486.522 | 487.3 |
| 93 | 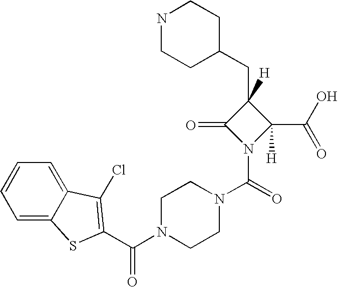 | Chiral | 519.019 | 519.22 |
| 94 | 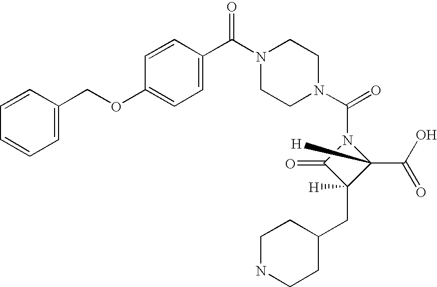 | Chiral | 534.61 | 535.33 |
| 95 | 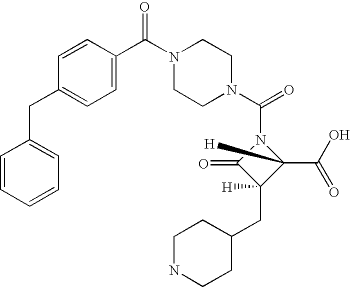 | Chiral | 518.611 | 519.33 |
| 96 | 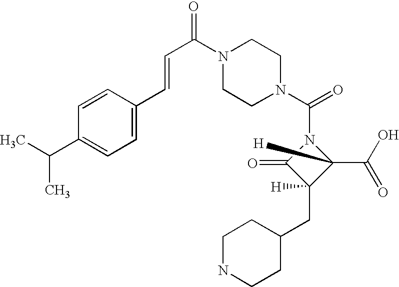 | Chiral | 496.604 | 497.36 |
| 97 | 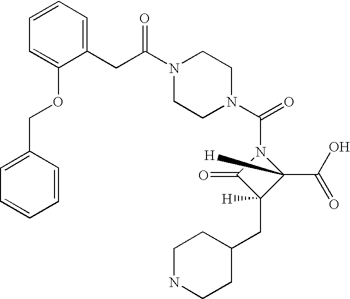 | Chiral | 548.636 | 549.37 |
| 98 | 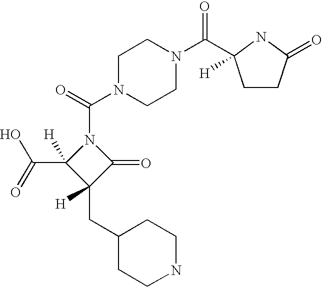 | Chiral | 435.478 | 436.3 |
| 99 | 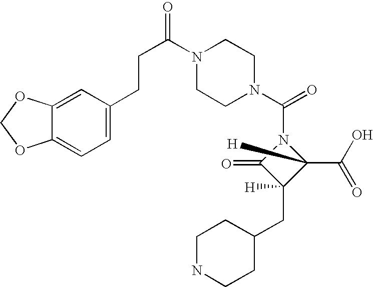 | Chiral | 500.549 | 501.32 |
| 100 | 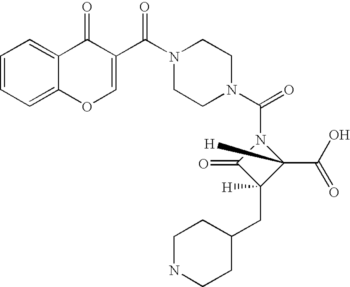 | Chiral | 496.517 | 497.29 |
| 101 | 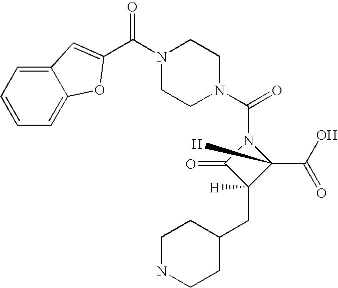 | Chiral | 468.507 | 469.33 |
| 102 | 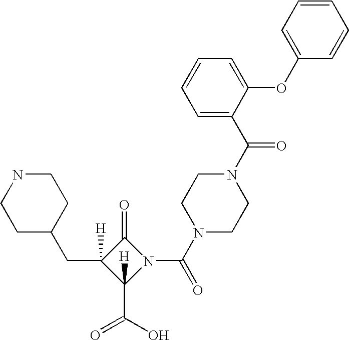 | Chiral | 520.583 | 521.32 |
| 103 | 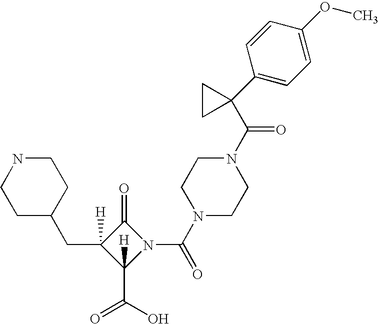 | Chiral | 498.577 | 499.33 |
| 104 | 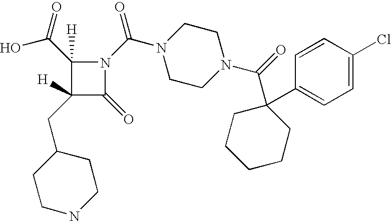 | Chiral | 545.076 | 545.34 |
| 105 | 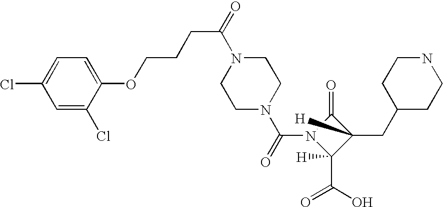 | Chiral | 555.456 | 555.27 |
| 106 | 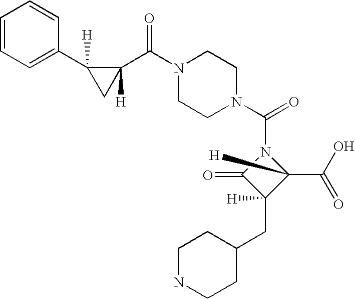 | Chiral | 468.551 | 469.37 |
| 107 | 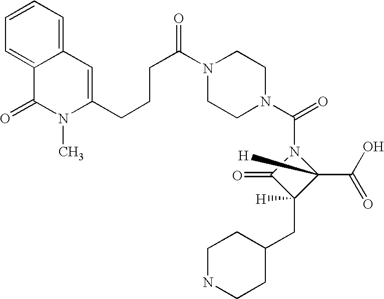 | Chiral | 551.64 | 552.38 |
| 108 | 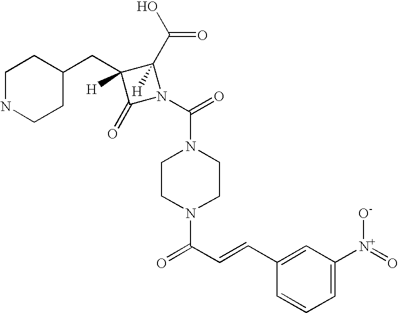 | Chiral | 499.521 | 500.29 |
| 109 | 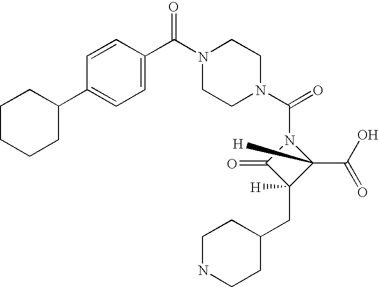 | Chiral | 510.631 | 511.36 |
| 110 | 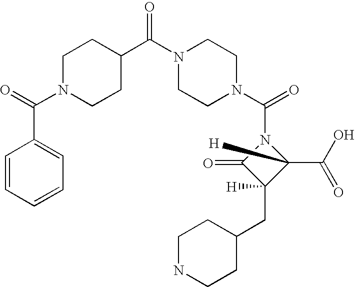 | Chiral | 539.629 | 540.37 |
| 111 | 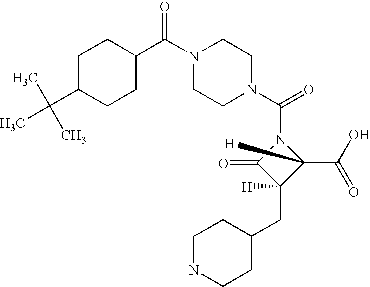 | Chiral | 490.641 | 491.42 |
| 112 | 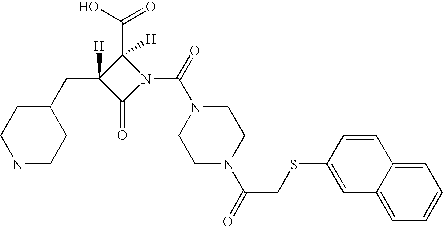 | Chiral | 524.639 | 525.29 |
| 113 | 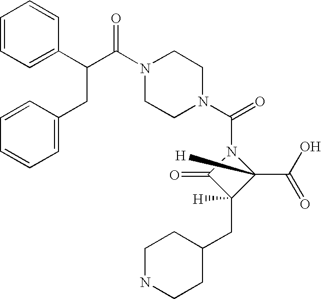 | Chiral | 532.637 | 533.36 |
| 114 | 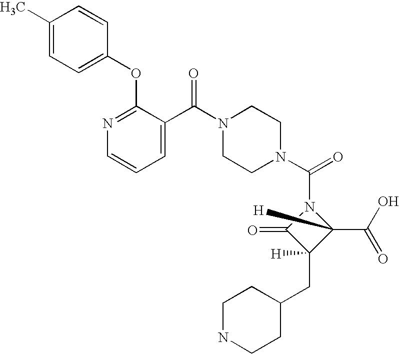 | Chiral | 535.598 | 536.34 |
| 115 | 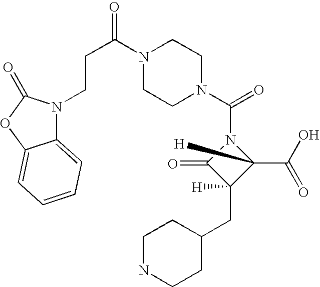 | Chiral | 513.548 | 514.3 |
| 116 | 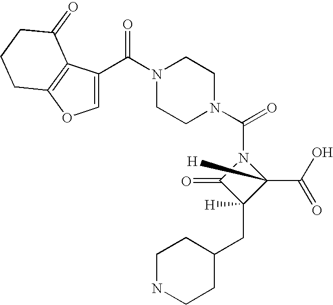 | Chiral | 486.522 | 487.3 |
| 117 | 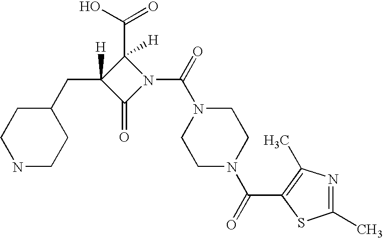 | Chiral | 463.556 | 464.32 |
| 118 | 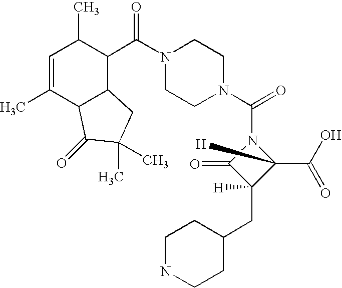 | Chiral | 538.641 | 539.37 |
| 119 | 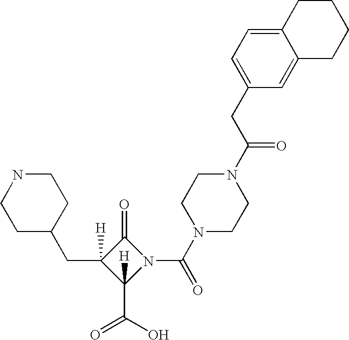 | Chiral | 496.604 | 497.36 |
| 120 | 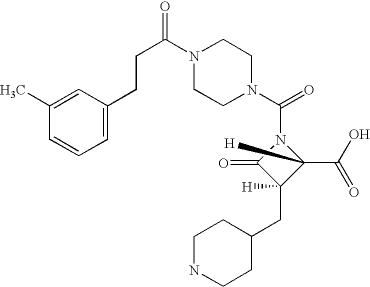 | Chiral | 470.567 | 471.36 |
| 121 | 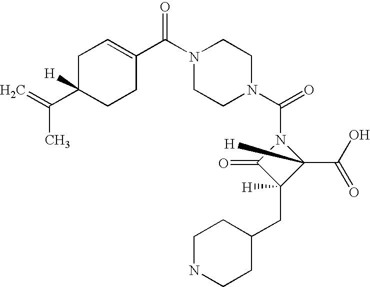 | Chiral | 472.582 | 473.34 |
| 122 | 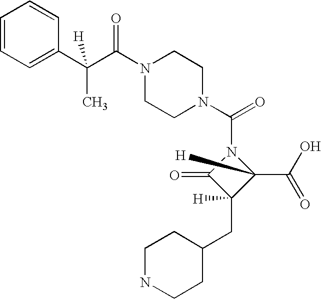 | Chiral | 456.54 | 457.34 |
| 123 | 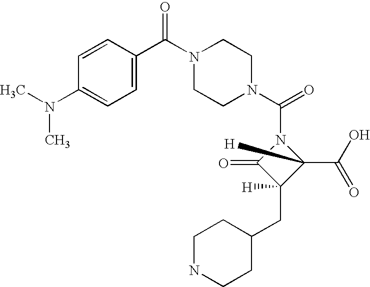 | Chiral | 471.555 | 473.35 |
| 124 | 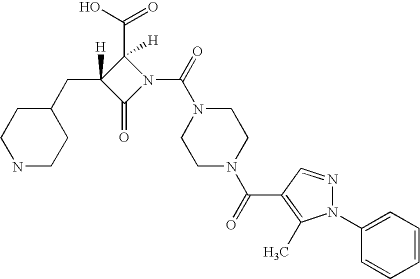 | Chiral | 508.576 | 509.32 |
| 125 | 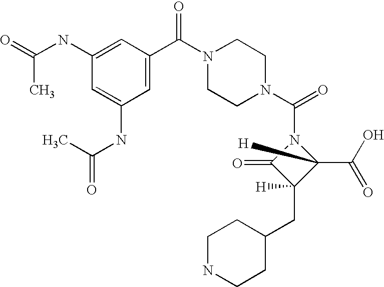 | Chiral | 542.59 | 543.35 |
| 126 | 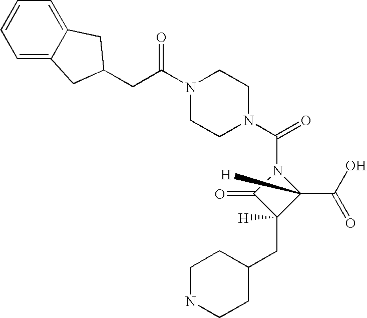 | Chiral | 482.578 | 483.37 |
| 127 | 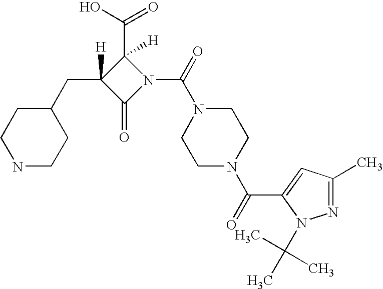 | Chiral | 488.585 | 489.38 |
| 128 | 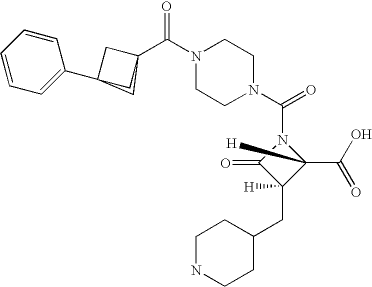 | Chiral | 494.589 | 495.35 |
| 129 | 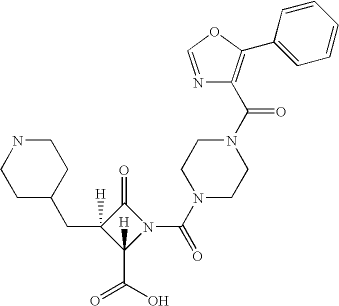 | Chiral | 495.533 | 496.32 |
| 130 | 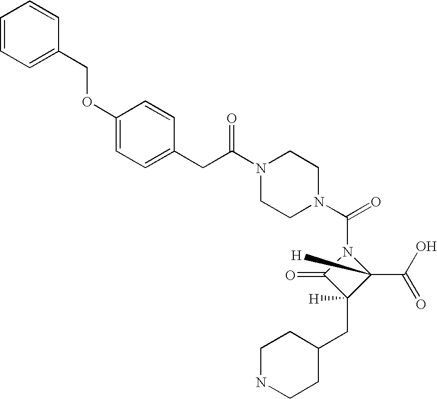 | Chiral | 548.636 | 549.38 |
| 131 | 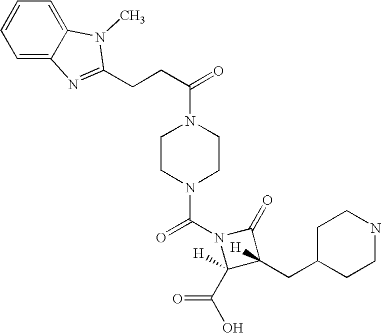 | Chiral | 510.592 | 511.35 |

### Examples 132 to 168

| Example No. | Structure | |
|---|---|---|
| 132 | 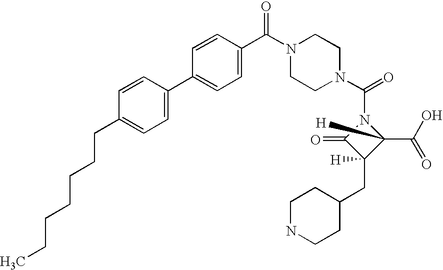 | Chiral |
| 133 | 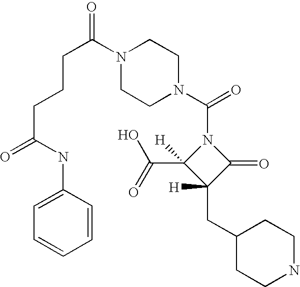 | Chiral |
| 134 | 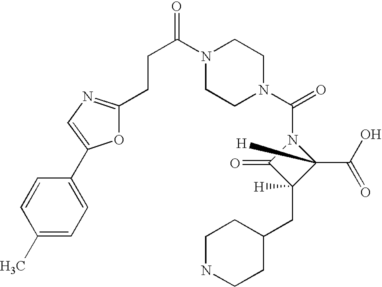 | Chiral |
| 135 | 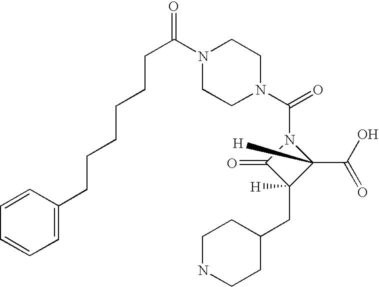 | Chiral |
| 136 | 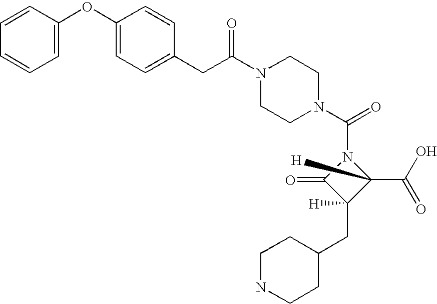 | Chiral |
| 137 | 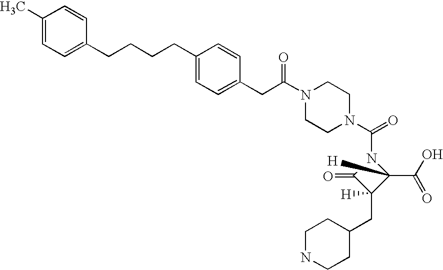 | Chiral |
| 138 | 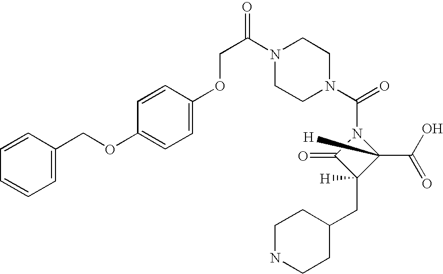 | Chiral |
| 139 | 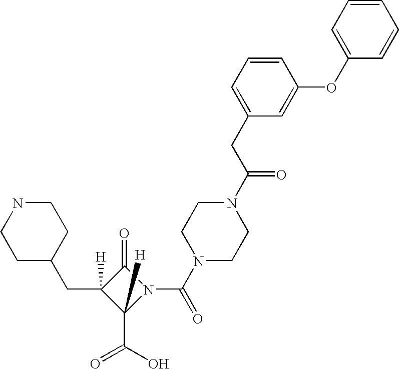 | Chiral |
| 140 | 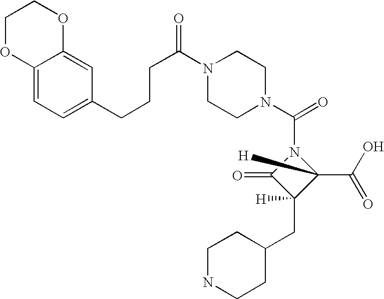 | Chiral |
| 141 | 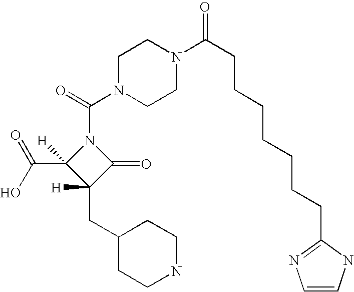 | Chiral |
| 142 | 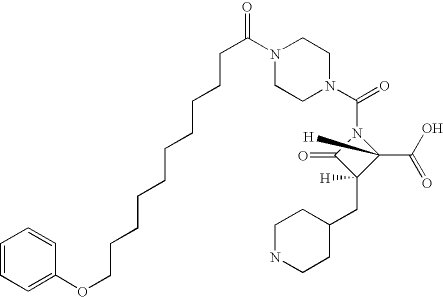 | Chiral |
| 143 | 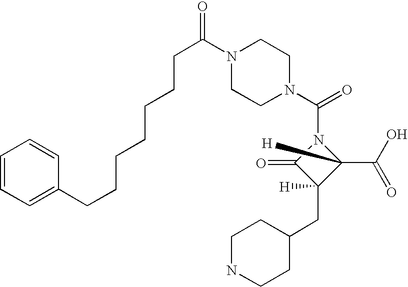 | Chiral |
| 144 | 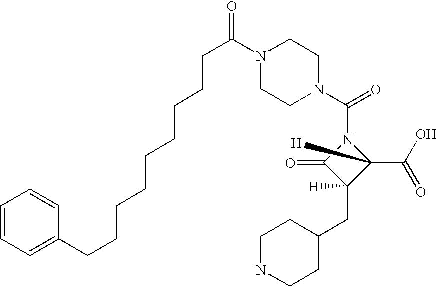 | Chiral |
| 145 | 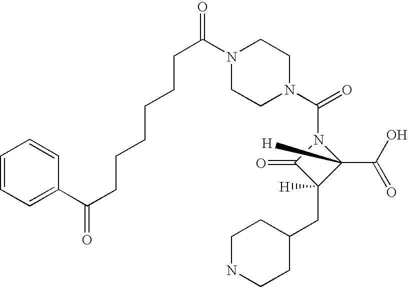 | Chiral |
| 146 | 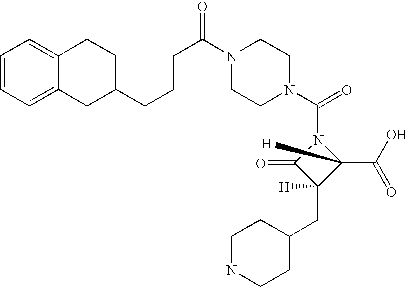 | Chiral |
| 147 | 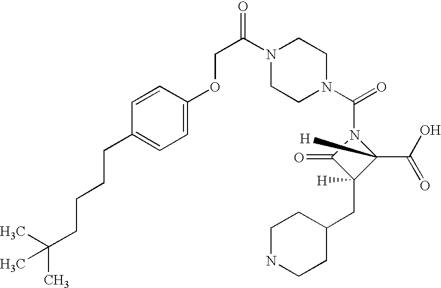 | Chiral |
| 148 | 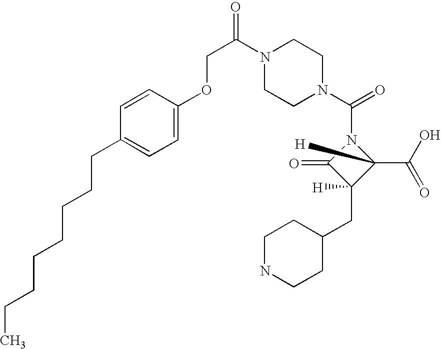 | Chiral |
| 149 | 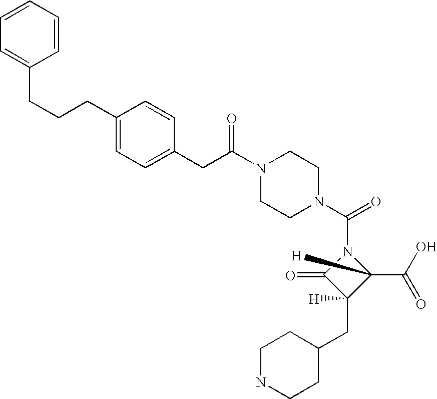 | Chiral |
| 150 | 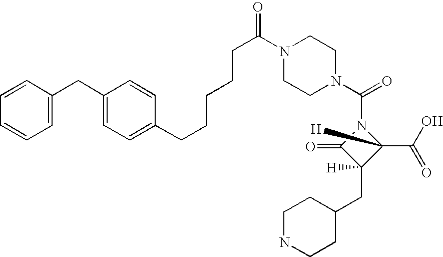 | Chiral |
| 151 | 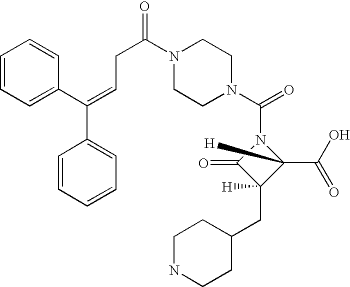 | Chiral |
| 152 | 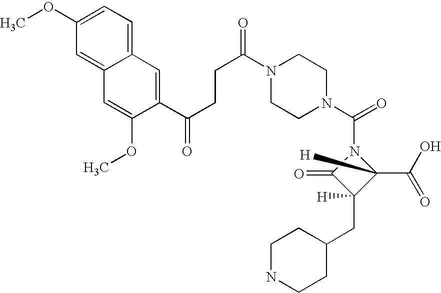 | Chiral |
| 153 | 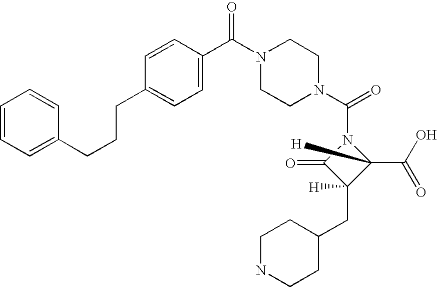 | Chiral |
| 154 | 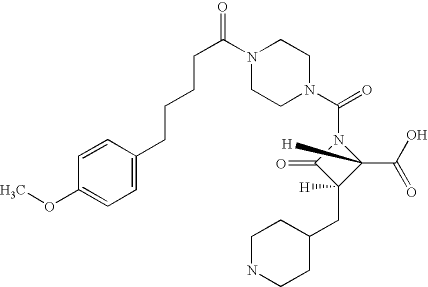 | Chiral |
| 155 | 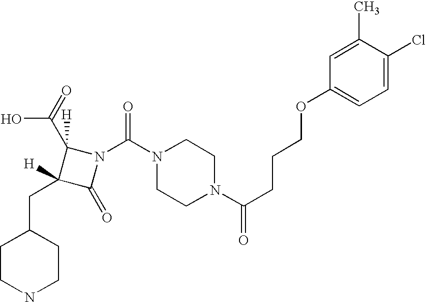 | Chiral |
| 156 | 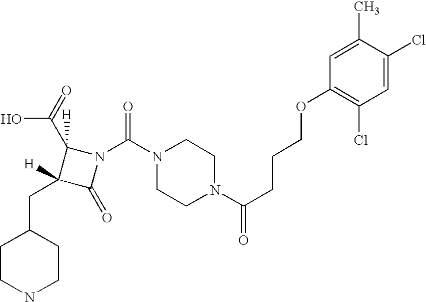 | Chiral |
| 157 | 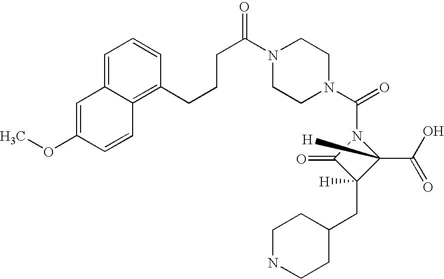 | Chiral |
| 158 | 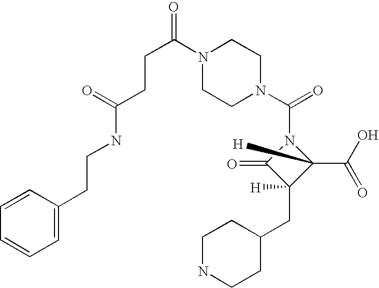 | Chiral |
| 159 | 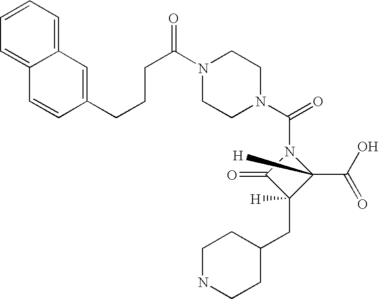 | Chiral |
| 160 | 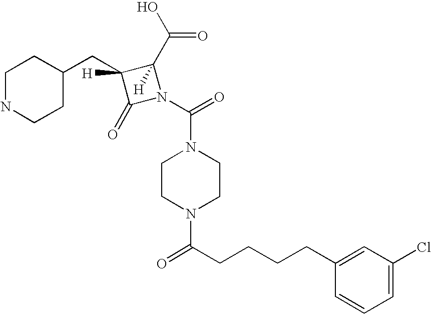 | Chiral |
| 161 | 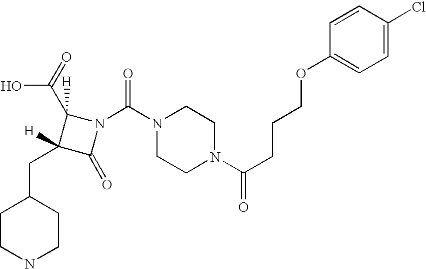 | Chiral |
| 162 | 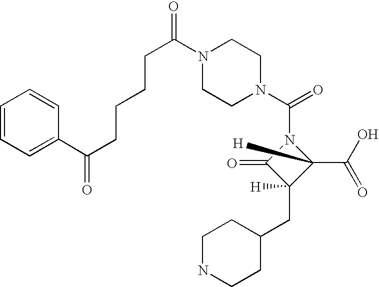 | Chiral |
| 163 | 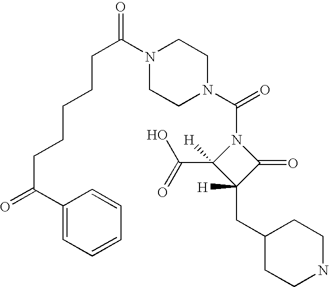 | Chiral |
| 164 | 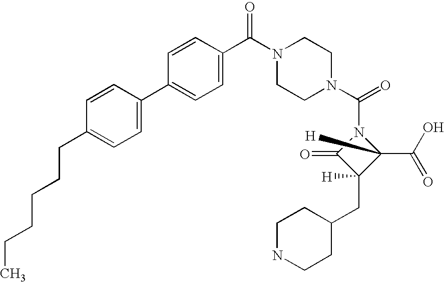 | Chiral |
| 165 | 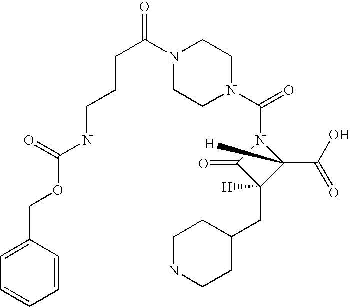 | Chiral |
| 166 | 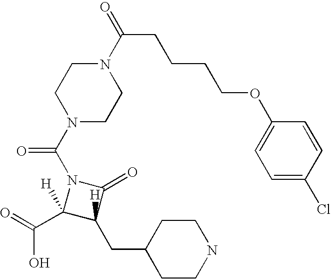 | Chiral |
| 167 | 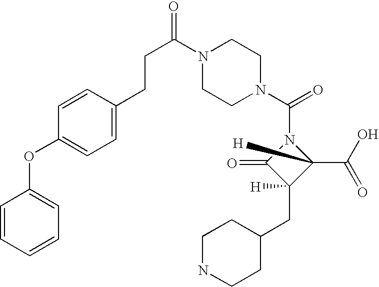 | Chiral |
| 168 | 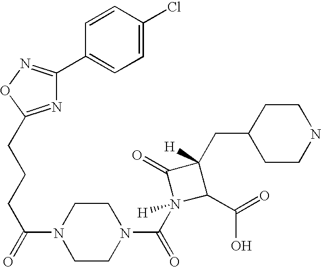 | Chiral |

The following additional Examples were prepared employing procedures set out hereinbefore in the reaction schemes and working Examples.

### Examples 169 to 191

| Example No. | Structure | |
|---|---|---|
| 169 | 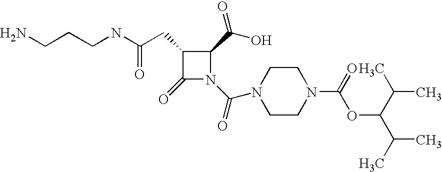 | Chiral |
| 170 | 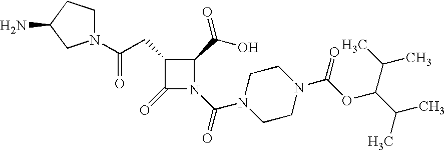 | Chiral |
| 171 | 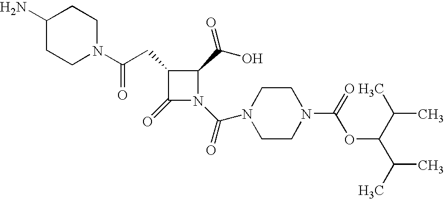 | Chiral |
| 172 | 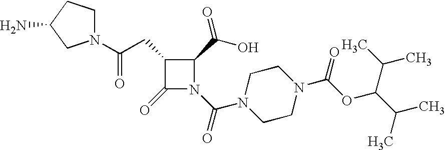 | Chiral |
| 173 | 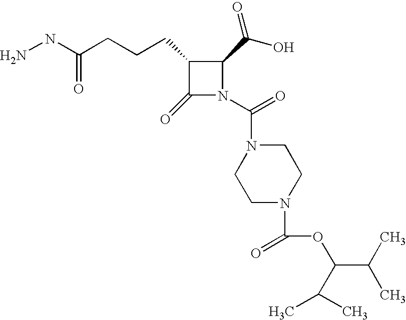 | Chiral |
| 174 | 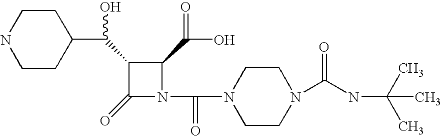 | |
| 175 | 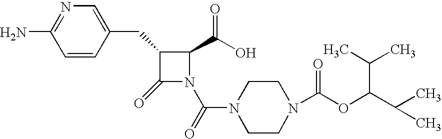 | Chiral |
| 176 | 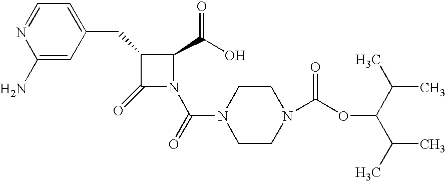 | Chiral |
| 177 | 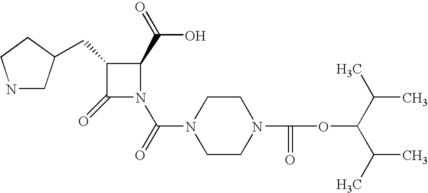 | |
| 178 | 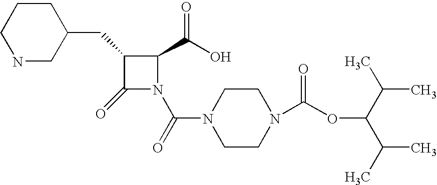 | |
| 179 | 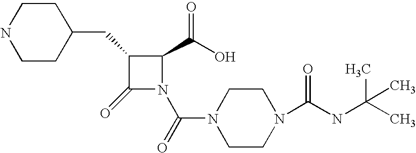 | Chiral |
| 180 | 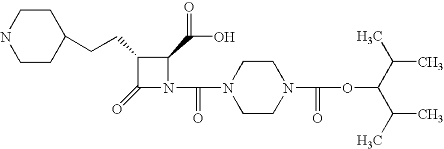 | Chiral |
| 181 | 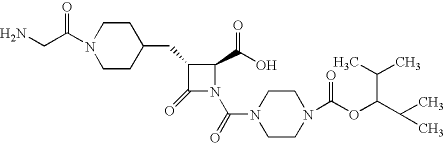 | Chiral |
| 182 | 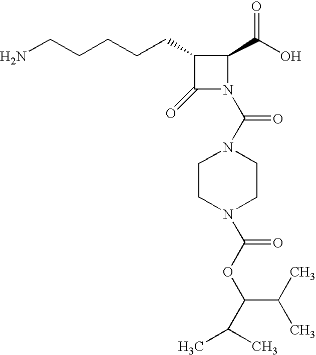 | Chiral |
| 183 | 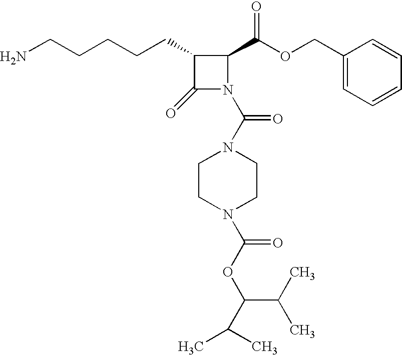 | Chiral |
| 184 | 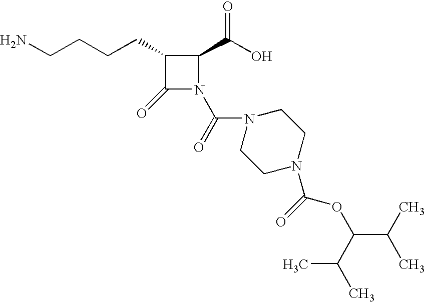 | Chiral |
| 185 | 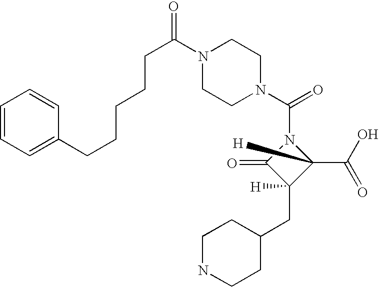 | Chiral |
| 186 | 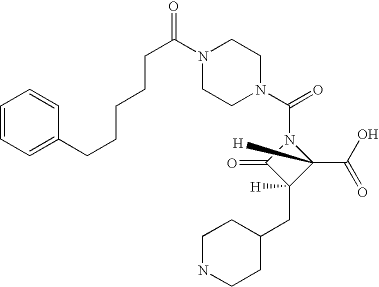 | Chiral |
| 187 | 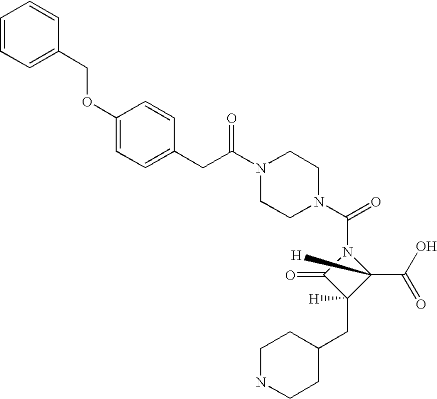 | Chiral |
| 188 | 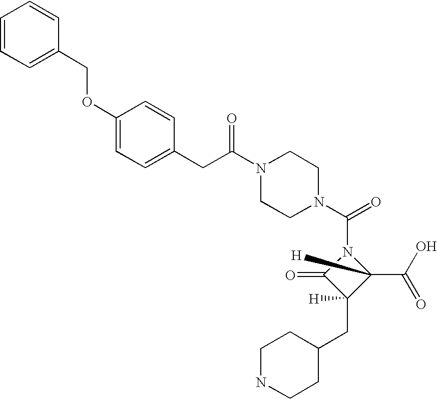 | Chiral |
| 189 | 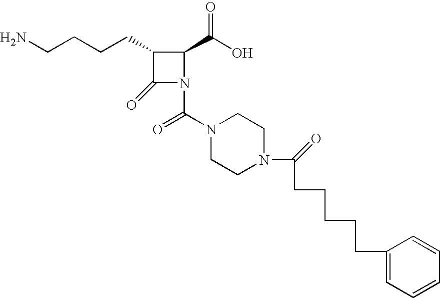 | Chiral |
| 190 | 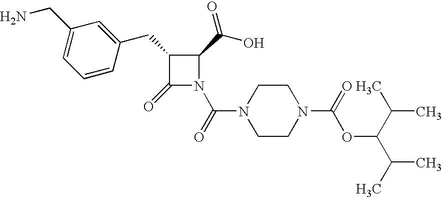 | Chiral |
| 191 | 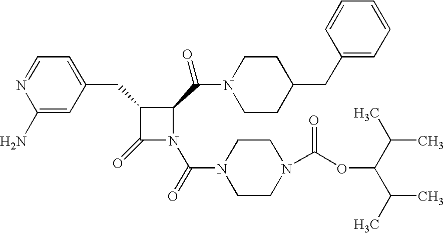 | Chiral |

What is claimed is:

1. A compound of the structure 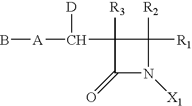
wherein:
D is H or OR^{a}
wherein R^{a} is H or alkyl;
A is a linear string of A¹, A², A³, A⁴, A⁵, A⁶, A⁷ and/or A⁸, in any order, such that A¹ may occur in the string from 0 to 6 times;
A² may occur in the string from 0 to 2 times;
A³, A⁴, A⁵, A⁶, A⁷ and/or A⁸ may each occur in the string 0 or 1 time, such that the total number of linear A groups is 0 to 6; 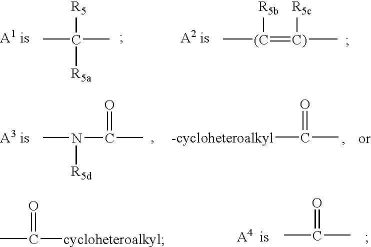
A⁵ is cycloalkyl;
A⁶ is aryl;
A⁷ is heteroaryl; and
A⁸ is cycloheteroalkyl,
wherein R₅ₐ, R₅ₐ¹, R_{5b}, R_{5c}, and R_{5d} are the same or different and are independently selected from H, alkyl, aryl, arylalkyl halo or nitro;
B is amino, aminoalkyl, aminoalkyl, aminocycloalkyl, cycloheteroalkyl, aryl, heteroaryl, alkylamino, carboxamido 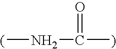
or cycloalkyl;
R₁ is hydrogen, carboxy, alkoxycarbonyl, A₂-aryl, 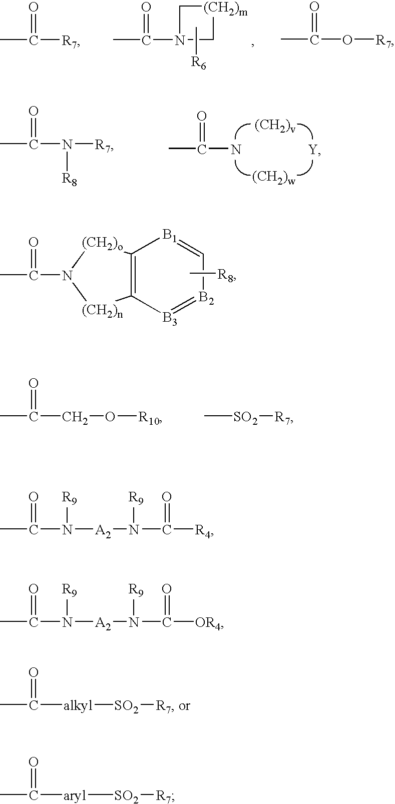
R₂ and R₃ are the same or different and are independently selected from hydrogen, or alkyl;
X₁ is 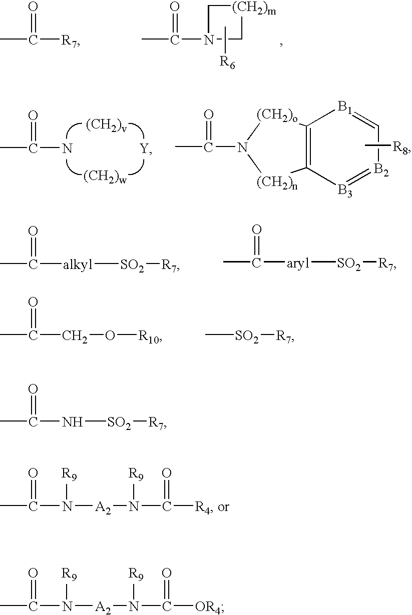
R₄ and R₅ are the same or different and are independently selected from hydrogen, alkyl, substituted alkyl, cycloalkyl, substituted cycloalkyl, A₂-cycloalkyl, A₂-substituted cycloalkyl, aryl, substituted aryl, A₂-aryl, A₂-substituted aryl, heteroaryl, A₂-heteroaryl, heterocycloalkyl, A₂-heterocycloalkyl, aryl-A₃-aryl, A₂-aryl-A₃-aryl, aryl-A₃-cycloalkyl, A₂-aryl-A₃-cycloalkyl, aryl-A₃-heteroaryl, A₂-aryl-A₃-heteroaryl, aryl-A₃-heterocycloalkyl, A₂-aryl-A₃-heterocycloalkyl, aryl-A₃-substituted aryl, A₂-aryl-A₃-substitued aryl, aryl-A₃-substituted cycloalkyl, A₂-aryl-A₃-substituted cycloalkyl, cycloalkyl-A₃-cycloalkyl, A₂-cycloalkyl-A₃-cycloalkyl, cycloalkyl-A₃-aryl, A₂-cycloalkyl-A₃-aryl, cycloalkyl-A₃-heteroaryl, A₂-cycloalkyl-A₃-heteroaryl, cycloalkyl-A₃-heterocycloalkyl, A₂-cycloalkyl-A₃-heterocycloalkyl, cycloalkyl-A₃-substituted cycloalkyl, A₂-cycloalkyl-A₃-substituted cycloalkyl, cycloalkyl-A₃-substituted aryl, A₂-cycloalkyl-A₃-substituted aryl, substituted cycloalkyl-A₃-cycloalkyl, A₂-substituted cycloalkyl-A₃-cycloalkyl, substituted cycloalkyl-A₃-substituted cycloalkyl, A₂-substituted cycloalkyl-A₃-substituted cycloalkyl, substituted cycloalkyl-A₃-aryl, A₂-substituted cycloalkyl-A₃-aryl, substituted cycloalkyl-A₃-heteroaryl, A₂-substituted cycloalkyl-A₃-heteroaryl, substituted cycloalkyl-A₃-heterocycloalkyl, A₂-substituted cycloalkyl-A₃-heterocycloalkyl, substituted cycloalkyl-A₃-substituted aryl, A₂-substituted cycloalkyl-A₃-substituted aryl, heteroaryl-A₃-heteroaryl, A₂-heteroaryl-A₃-heteroaryl, heteroaryl-A₃-cycloalkyl, A₂-heteroaryl-A₃-cycloalkyl, heteroaryl-A₃-substituted cycloalkyl, A₂-heteroaryl-A₃-substituted cycloalkyl, heteroaryl-A₃-aryl, A₂-heteroaryl-A₃-aryl, heteroaryl-A₃-heterocycloalkyl, A₂-heteroaryl-A₃-heterocycloalkyl, heteroaryl-A₃-substituted aryl, A₂-heteroaryl-A₃-substituted aryl, heterocycloalkyl-A₃-heterocycloalkyl, A₂-heterocycloalkyl-A₃-heterocycloalkyl, heterocycloalkyl-A₃-cycloalkyl, A₂-heterocycloalkyl-A₃-cycloalkyl, heterocycloalkyl-A₃-substituted cycloalkyl, A₂-heterocycloalkyl-A₃-substituted cycloalkyl, heterocycloalkyl-A₃-aryl, A₂-heterocycloalkyl-A₃-aryl, heterocycloalkyl-A₃-substituted aryl, A₂-heterocycloalkyl-A₃-substituted aryl, heterocycloalkyl-A₃-heteroaryl, A₂-heterocycloalkyl-A₃-heteroaryl, substituted aryl-A₃-substituted aryl, A₂-substituted aryl-A₃-substituted aryl, substituted aryl-A₃-cycloalkyl, A₂-substituted aryl-A₃-cycloalkyl, substituted aryl-A₃-substituted cycloalkyl, A₂-substituted aryl-A₃-substituted cycloalkyl, substituted aryl-A₃-aryl, A₂-substituted aryl-A₃-aryl, substituted aryl-A₃-heteroaryl, A₂-substituted aryl-A₃-heteroaryl, substituted aryl-A₃-heterocycloalkyl, and A₂-substituted aryl-A₃-heterocycloalkyl;
R₆ is hydrogen, alkyl, substituted alkyl, cycloalkyl, substituted cycloalkyl, A₂-cycloalkyl, A₂-substituted cycloalkyl, aryl, substituted aryl, A₂-aryl, A₂-substituted aryl, aryl-A₃-aryl, A₂-aryl-A₃-aryl, heteroaryl, A₂-heteroaryl, heterocycloalkyl, A₂-heterocycloalkyl, aryl-A₃-cycloalkyl, A₂-aryl-A₃-cycloalkyl, aryl-A₃-heteroaryl, A₂-aryl-A₃-heteroaryl, aryl-A₃-heterocycloalkyl, A₂-aryl-A₃-heterocycloalkyl, carboxy, alkoxycarbonyl, aryloxycarbonyl, 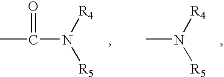
alkoxycarbonylamino, aryloxycarbonylamino, arylcarbonylamino, —N(alkyl)(alkoxycarbonyl), —N(alkyl)(aryloxycarbonyl), alkylcarbonylamino, —N(alkyl)(alkylcarbonyl), or —N(alkyl)(arylcarbonyl);
m is an integer from 1 to 5;
Y is O, S, N-4, N—SO₂—R₇, 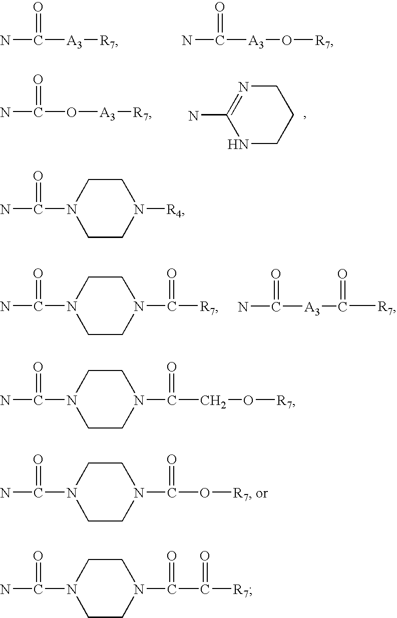
R₇ is hydrogen, alkyl, substituted alkyl, cycloalkyl, substituted cycloalkyl, A₂-cycloalkyl, A₂-substituted cycloalkyl, aryl, substituted aryl, A₂-aryl, A₂-substituted aryl, heteroaryl, A₂-heteroaryl, heterocycloalkyl, A₂-heterocycloalkyl, aryl-A₃-aryl, A₂-aryl-A₃-aryl, aryl-A₃-cycloalkyl, A₂-aryl-A₃-cycloalkyl, aryl-A₃-heteroaryl, A₂-aryl-A₃-heteroaryl, aryl-A₃-heterocycloalkyl, A₂-aryl-A₃-heterocycloalkyl, aryl-A₃-substituted aryl, A₂-aryl-A₃-substitued aryl, aryl-A₃-substituted cycloalkyl, A₂-aryl-A₃-substituted cycloalkyl, cycloalkyl-A₃-cycloalkyl, A₂-cycloalkyl-A₃-cycloalkyl, cycloalkyl-A₃-aryl, A₂-cycloalkyl-A₃-aryl, cycloalkyl-A₃-heteroaryl, A₂-cycloalkyl-A₃-heteroaryl, cycloalkyl-A₃-heterocycloalkyl, A₂-cycloalkyl-A₃-heterocycloalkyl, cycloalkyl-A₃-substituted cycloalkyl, A₂-cycloalkyl-A₃-substituted cycloalkyl, cycloalkyl-A₃-substituted aryl, A₂-cycloalkyl-A₃-substituted aryl, substituted cycloalkyl-A₃-cycloalkyl, A₂-substituted cycloalkyl-A₃-cycloalkyl, substituted cycloalkyl-A₃-substituted cycloalkyl, A₂-substituted cycloalkyl-A₃-substituted cycloalkyl, substituted cycloalkyl-A₃-aryl, A₂-substituted cycloalkyl-A₃-aryl, substituted cycloalkyl-A₃-heteroaryl, A₂-substituted cycloalkyl-A₃-heteroaryl, substituted cycloalkyl-A₃-heterocycloalkyl, A₂-substituted cycloalkyl-A₃-heterocycloalkyl, substituted cycloalkyl-A₃-substituted aryl, A₂-substituted cycloalkyl-A₃-substituted aryl, heteroaryl-A₃-heteroaryl, A₂-heteroaryl-A₃-heteroaryl, heteroaryl-A₃-cycloalkyl, A₂-heteroaryl-A₃-cycloalkyl, heteroaryl-A₃-substituted cycloalkyl, A₂-heteroaryl-A₃-substituted cycloalkyl, heteroaryl-A₃-aryl, A₂-heteroaryl-A₃-aryl, heteroaryl-A₃-heterocycloalkyl, A₂-heteroaryl-A₃-heterocycloalkyl, heteroaryl-A₃-substituted aryl, A₂-heteroaryl-A₃-substituted aryl, heterocycloalkyl-A₃-heterocycloalkyl, A₂-heterocycloalkyl-A₃-heterocycloalkyl, heterocycloalkyl-A₃-cycloalkyl, A₂-heterocycloalkyl-A₃-cycloalkyl, heterocycloalkyl-A₃-substituted cycloalkyl, A₂-heterocycloalkyl-A₃-substituted cycloalkyl, heterocycloalkyl-A₃-aryl, A₂-heterocycloalkyl-A₃-aryl, heterocycloalkyl-A₃-substituted aryl, A₂-heterocycloalkyl-A₃-substituted aryl, heterocycloalkyl-A₃-heteroaryl, A₂-heterocycloalkyl-A₃-heteroaryl, substituted aryl-A₃-substituted aryl, A₂-substituted aryl-A₃-substituted aryl, substituted aryl-A₃-cycloalkyl, A₂-substituted aryl-A₃-cycloalkyl, substituted aryl-A₃-substituted cycloalkyl, A₂-substituted aryl-A₃-substituted cycloalkyl, substituted aryl-A₃-aryl, A₂-substituted aryl-A₃-aryl, substituted aryl-A₃-heteroaryl, A₂-substituted aryl-A₃-heteroaryl, substituted aryl-A₃-R₄ heterocycloalkyl, A₂-substituted aryl-A₃-heterocycloalkyl, 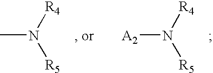
n and o are independently one or two provided that the sum of n plus o is two or three;
v and w are independently one, two, or three provided that the sum of v plus w is three, four, or five;
R₉ is hydrogen, halo, amino, —NH(lower alkyl), —N(lower alkyl)₂, nitro, alkyl, substituted alkyl, alkoxy, hydroxy, aryl, substituted aryl, A₂-aryl, A₂-substituted aryl, aryl-A₃-aryl, A₂-aryl-A₃-aryl, cycloalkyl, substituted cycloalkyl, A₂-cycloalkyl, A₂-substituted cycloalkyl, heteroaryl, A₂-heteroaryl, heterocycloalkyl, A₂-heterocycloalkyl, aryl-A₃-cycloalkyl, A₂-aryl-A₃-cycloalkyl, aryl-A₃-heteroaryl, A₂-aryl-A₃-heteroaryl, aryl-A₃-heterocycloalkyl, or A₂-aryl-A₃-heterocycloalkyl;
B₁, B₂ and B₃ are each CH, or two of B₁, B₂ and B₃ are CH and the other is N, or one of B₁, B₂ and B₃ is CH and the other two are N;
R₉ is hydrogen or lower alkyl;
R₁₀ is alkyl, substituted alkyl, alkyl-O-alkyl, alkyl-O-alkyl-O-alkyl, cycloalkyl, substituted cycloalkyl, A₂-cycloalkyl, A₂-substituted cycloalkyl, aryl, substituted aryl, A₂-aryl, A₂-substituted aryl, aryl-A₃-aryl, A₂-aryl-A₃-aryl, heteroaryl, A₂-heteroaryl, heterocycloalkyl, A₂-heterocycloalkyl, aryl-A₃-cycloalkyl, A₂-aryl-A₃-cycloalkyl, aryl-A₃-heteroaryl, A₂-aryl-A₃-heteroaryl, aryl-A₃-heterocycloalkyl or A₂-aryl-A₃-heterocycloalkyl;
R₂₁ and R₂₂ are the same or different and are independently selected from hydrogen, alkyl, substituted alkyl, cycloalkyl, substituted cycloalkyl, A₂-cycloalkyl, A₂-substituted cycloalkyl, A₂-aryl, and A₂-substituted aryl;
p is an integer from 2 to 6;
q is an integer from 1 to 6;
r is zero, 1, 2 or 3;
s is 1, 2 or 3;
t is 1, 2, 3 or 4;
u is 1, 2 or 3;
A₂ is an alkylene or a substituted alkylene bridge of 1 to 10 carbons, an alkenyl or substituted alkenyl bridge of 2 to 10 carbons having one or more double bonds, or an alkynyl or substituted alkynyl bridge of 2 to 10 carbons having one or more triple bonds;
A₃ is a bond, an alkylene or a substituted alkylene bridge of 1 to 10 carbons, an alkenyl or substituted alkenyl bridge of 2 to 10 carbons having one or more double bonds, an alkynyl or substituted alkynyl bridge of 2 to 10 carbons having one or more triple bonds, 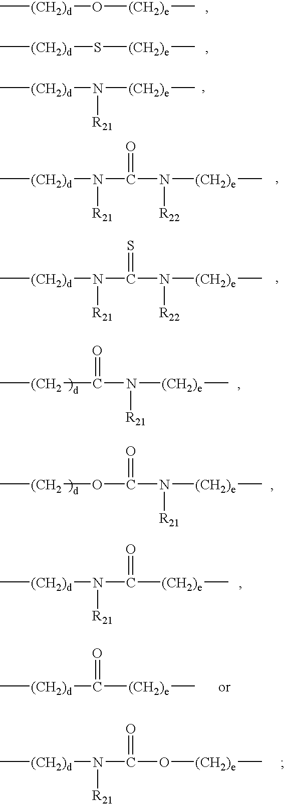
d and e are independently selected from zero and an integer from 1 to 10 provided that the sum of d plus e is no greater than 10;
and an inner salt or a pharmaceutically acceptable salt thereof, a hydrolyzable ester thereof, or a solvate thereof, with the provisos that
(a) where R₁ is COOZ, where Z is 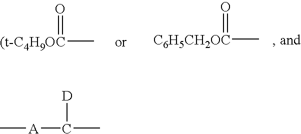
is (CH₂)_{q}, then B is other than amino or R₂₀—NH— where R₂₀ is alkyl, cycloalkyl, A₂-cycloalkyl or A₂-aryl;
(b) where R₁ is 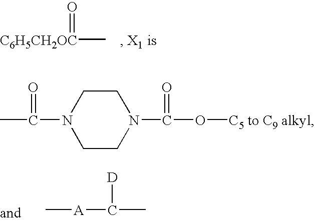
is other than 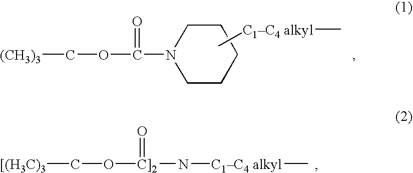
(3) amino C₁-C₅ alkyl,
(4) C₁-C₄ alkylamino C₁-C₅ alkyl, or
(5) piperidyl.

2. The compound as defined in Claim 1 wherein R₃ and R₂ are each H.

3. The compound as defined in Claim 1 wherein R₁ is carboxy or arylalkoxycarbonyl.

4. The compound as defined in Claim 1 wherein R₁ is carboxy,

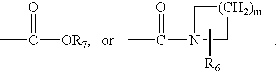

5. The compound as defined in Claim 4 wherein R₇ is substituted alkyl, R₆ is substituted alkyl and m is 2.

6. The compound as defined in Claim 1 wherein X₁ is 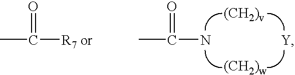
where Y is

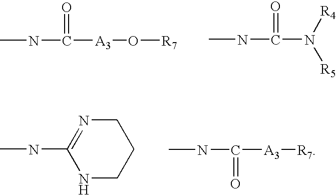

7. The compound as defined in Claim 6 wherein

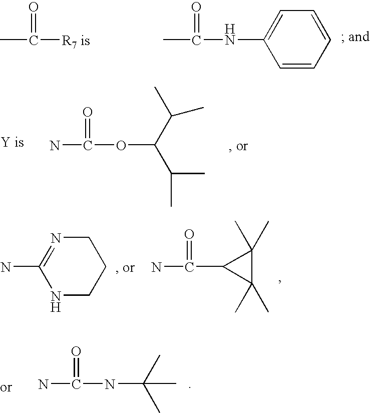

8. The compound as defined in Claim 1 wherein A is a bond, heteroaryl, (alkylene) 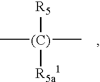 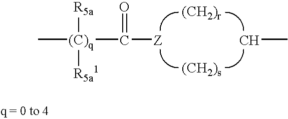
where Z is CH or N, r=0 to 3, s=0 to 3, (alkylcarbonyl cycloheteroalkyl), carbonyl cycloheteroalkyl, 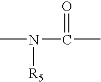
(aminocarbonyl), 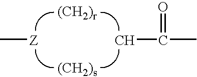
(cycloheteroalkylcarbonyl), 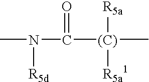
(aminocarbonylalkyl), or

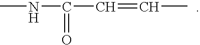

9. The compound as defined in Claim 1 wherein B is heteroaryl, cycloheteroaryl, alkylcycloheteroalkyl, amino, alkylamino, dialkylamino or aminoalkyl.

10. The compound as defined in Claim 1 wherein 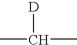
is —CH₂.

11. The compound as defined in Claim 1 wherein X₁ is 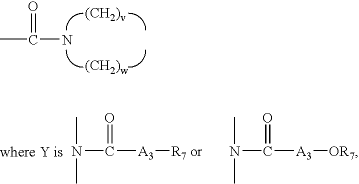
wherein A₃ is a bond, and R₇ is alkyl, cycloalkyl, aryl or arylalkyl, or Y is

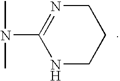

12. The compound as defined in Claim 10 where X₁ includes the moiety

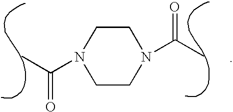

13. The compound as defined in Claim 1 wherein
R₁ is carboxy, benzyloxycarbonyl, 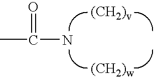
where Y is 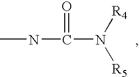
R₂ is H, R₃ is H, D is H, A is 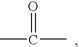
a bond, alkylene, -cycloheteroalkyl 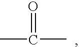
or heteroaryl, and B is amino, cycloheteroalkyl, or heteroaryl.

14. The compound as defined in Claim 13 wherein R₇ is 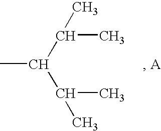
is pyridyl, B is 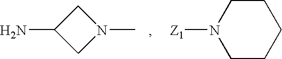
where Z₁ is H, NH₂CO or alkyl, or B is

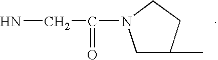

15. The compound as defined in Claim 1 having the structure

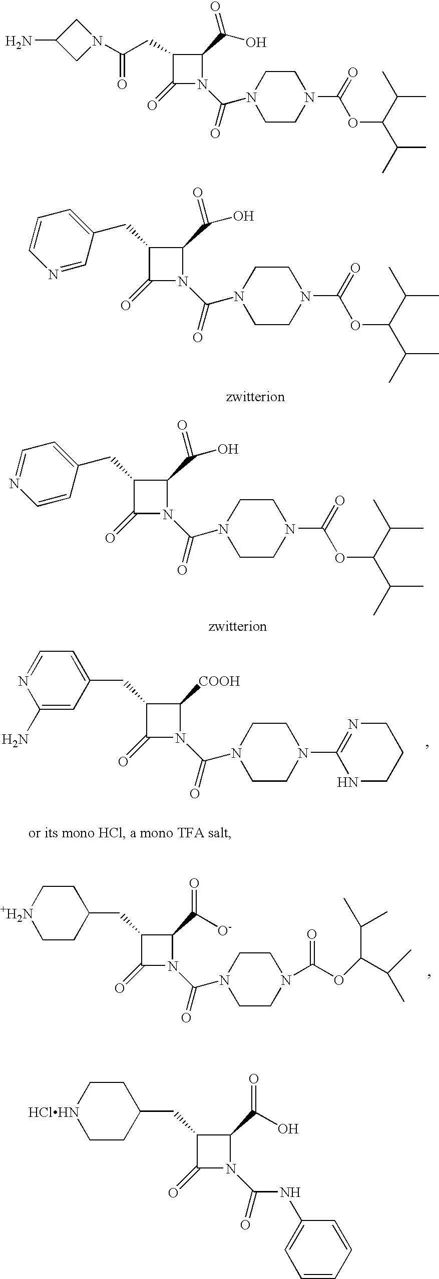

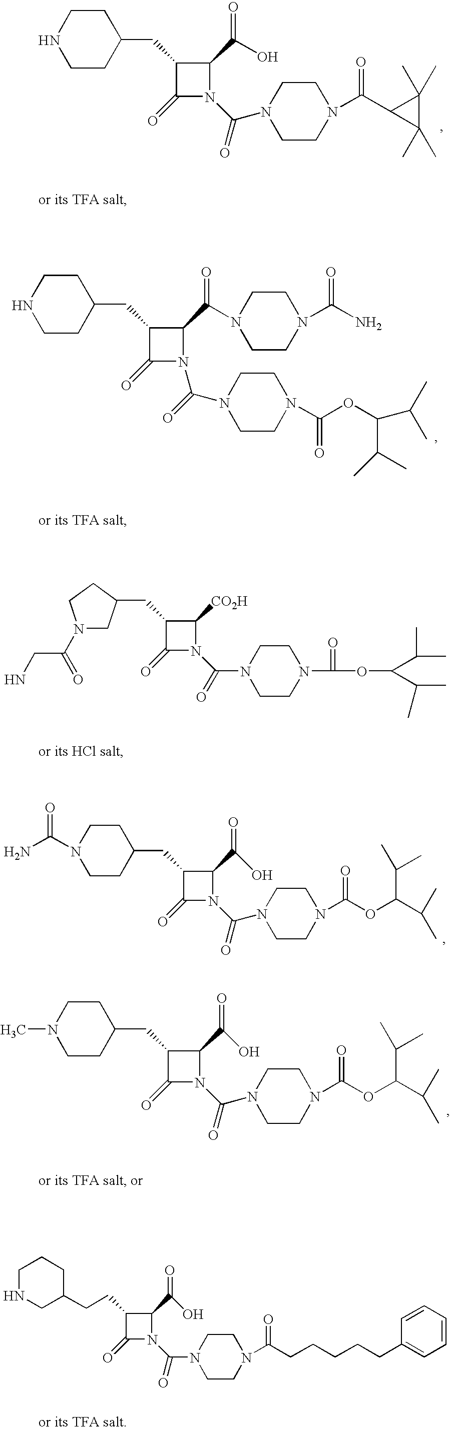

16. A compound of the formula 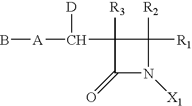
or including an inner salt thereof, or a pharmaceutically acceptable salt thereof, or a hydrolyzable ester thereof, or a solvate thereof wherein:
B, A, D, R₁, R₂ and R₃ are as defined in Claim 1; 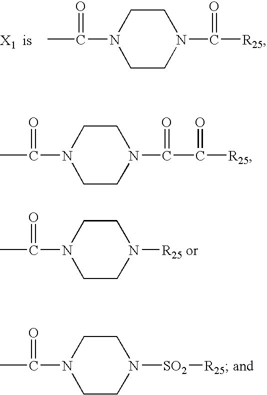
R₂₅ is a spacer terminating in a lipophilic group wherein said spacer comprises groups of 3 or more atoms or groups of 2 or more atoms and a phenylene, substituted phenylene, cycloalklene, heteroarylene, or heterocycloalkylene ring and said lipophilic terminating group is aryl, substituted aryl, cycloalkyl, heteroaryl, or heterocycloalkyl.

17. A compound of Claim 16 wherein R₂₅ is selected from the group

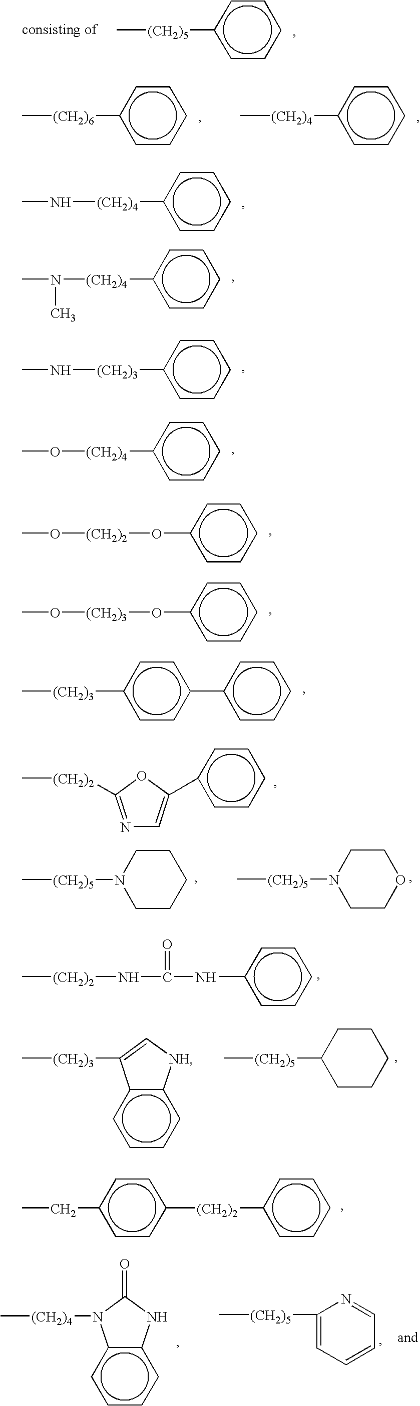

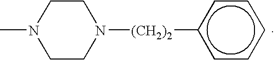

18. A compound of Claim 17 wherein R₂₅ is selected from the group consisting of

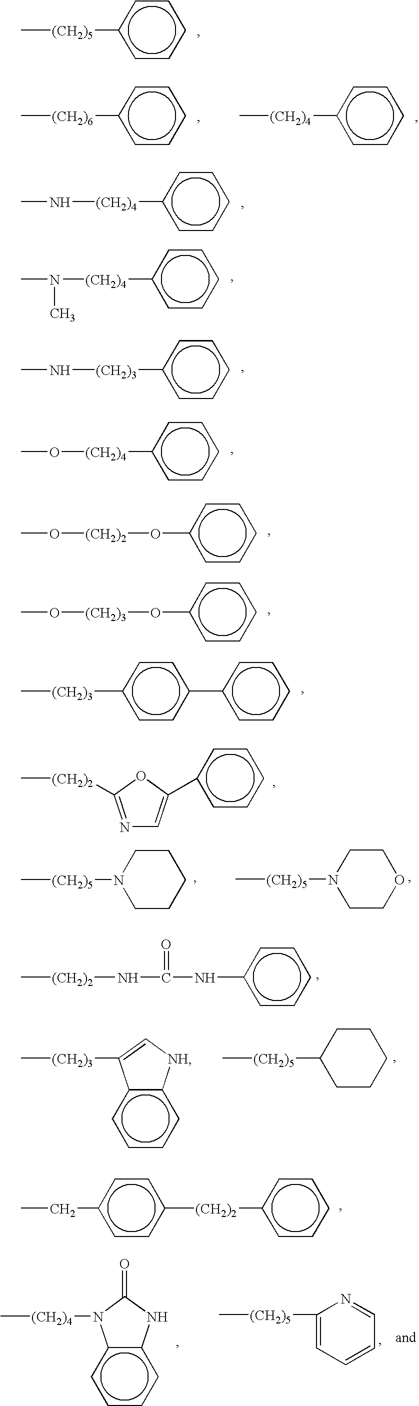

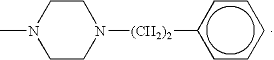

19. A compound of Claim 17 wherein

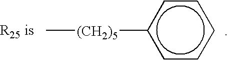

20. The compound as defined in Claim 16 having the following structure:

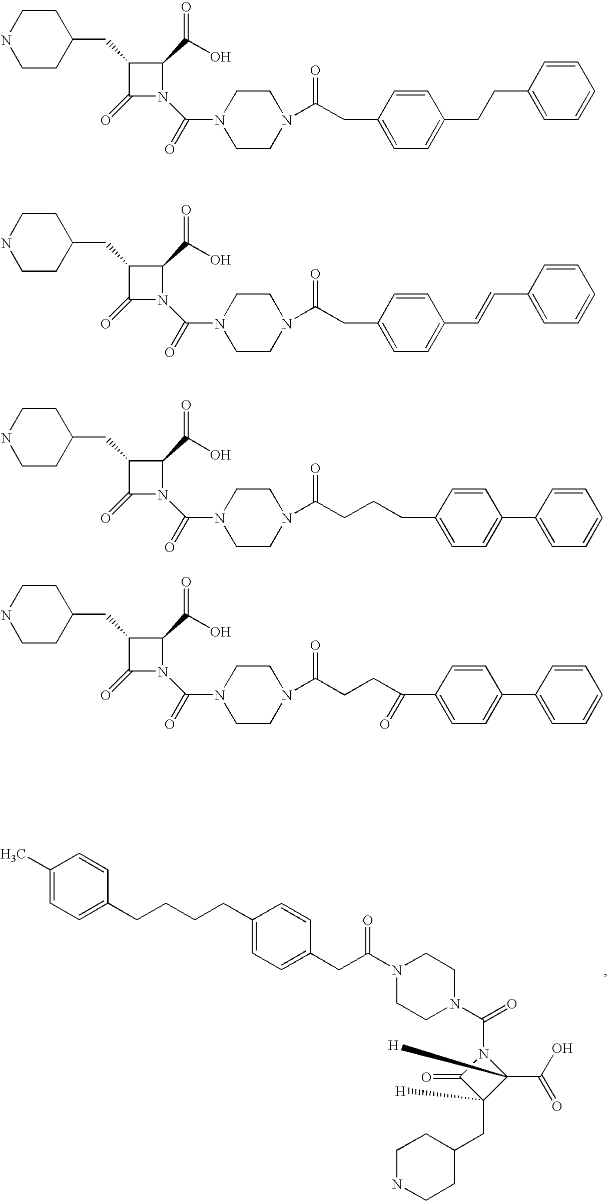

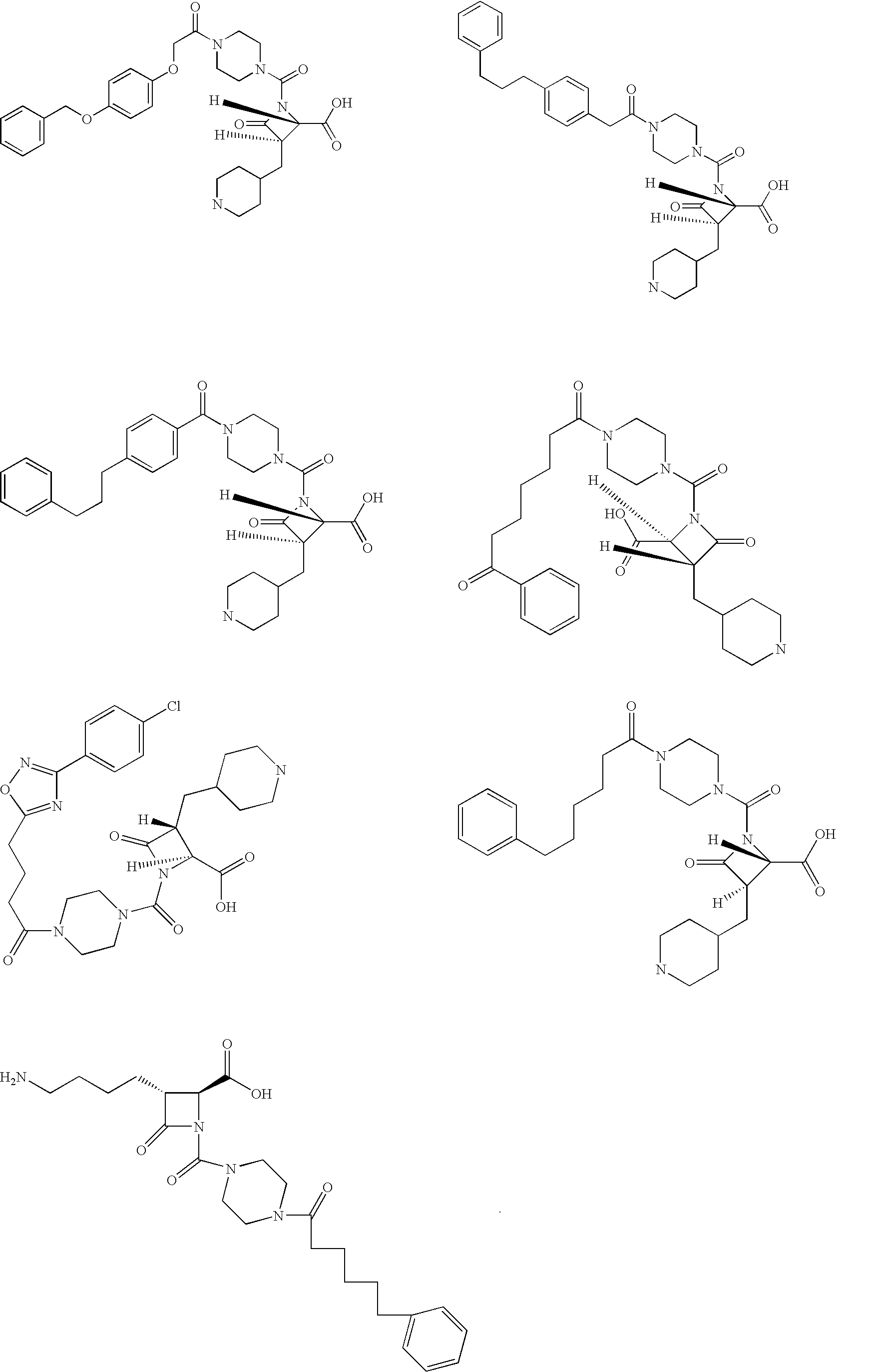

21. A pharmaceutical composition comprising a compound as defined in Claim 1 and a pharmaceutically acceptable carrier therefor.

22. A method for treating and/or preventing medical conditions in a mammalian species related to tryptase, thrombin, trypsin, Factor Xa, Factor VIIa, or urokinase-type plasminogen activator and/or for treating and/or preventing asthma or allergic rhinitis and/or for treating chronic asthma, which comprises administering a mammalian species a therapeutically effective amount of a compound of the structure 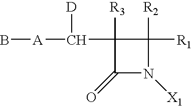
wherein:
D is H or OR^{a}
wherein R^{a} is H or alkyl;
A is a linear string of A¹, A², A³, A⁴, A⁵, A⁶, A⁷ and/or A⁸, in any order, such that A¹ may occur in the string from 0 to 6 times;
A² may occur in the string from 0 to 2 times;
A³, A⁴, A⁵, A⁶, A⁷ and/or A⁸ may each occur in the string 0 or 1 time, such that the total number of linear A groups is 0 to 6; 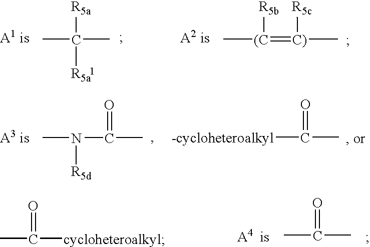
A⁵ is cycloalkyl;
A⁶ is aryl;
A⁷ is heteroaryl; and
A⁸ is cycloheteroalkyl;
wherein R₅ₐ, R₅ₐ¹, R_{5b}, R_{5c}, and R_{5d} are the same or different and are independently selected from H, alkyl, aryl, arylalkyl halo or nitro;
B is amino, aminoalkyl, aminoalkyl, aminocycloalkyl, cycloheteroalkyl, aryl, heteroaryl, alkylamino, carboxamido 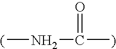
or cycloalkyl;
R₁ is hydrogen, carboxy, alkoxycarbonyl, A₂-aryl, alkyl, 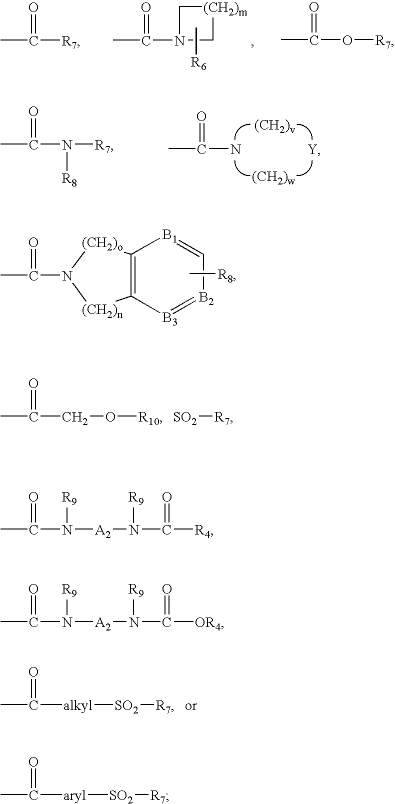
R₂ and R₃ are the same or different and are independently selected from hydrogen, or alkyl; 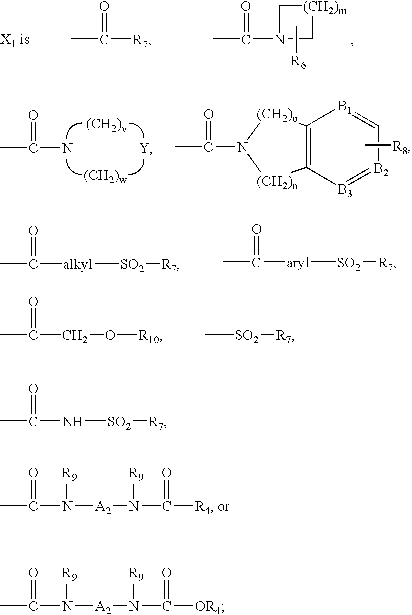
R₄ and R₅ are the same or different and are independently selected from hydrogen, alkyl, substituted alkyl, cycloalkyl, substituted cycloalkyl, A₂-cycloalkyl, A₂-substituted cycloalkyl, aryl, substituted aryl, A₂-aryl, A₂-substituted aryl, heteroaryl, A₂-heteroaryl, heterocycloalkyl, A₂-heterocycloalkyl, aryl-A₃-aryl, A₂-aryl-A₃-aryl, aryl-A₃-cycloalkyl, A₂-aryl-A₃-cycloalkyl, aryl-A₃-heteroaryl, A₂-aryl-A₃-heteroaryl, aryl-A₃-heterocycloalkyl, A₂-aryl-A₃-heterocycloalkyl, aryl-A₃-substituted aryl, A₂-aryl-A₃-substitued aryl, aryl-A₃-substituted cycloalkyl, A₂-aryl-A₃-substituted cycloalkyl, cycloalkyl-A₃-cycloalkyl, A₂-cycloalkyl-A₃-cycloalkyl, cycloalkyl-A₃-aryl, A₂-cycloalkyl-A₃-aryl, cycloalkyl-A₃-heteroaryl, A₂-cycloalkyl-A₃-heteroaryl, cycloalkyl-A₃-heterocycloalkyl, A₂-cycloalkyl-A₃-heterocycloalkyl, cycloalkyl-A₃-substituted cycloalkyl, A₂-cycloalkyl-A₃-substituted cycloalkyl, cycloalkyl-A₃-substituted aryl, A₂-cycloalkyl-A₃-substituted aryl, substituted cycloalkyl-A₃-cycloalkyl, A₂-substituted cycloalkyl-A₃-cycloalkyl, substituted cycloalkyl-A₃-substituted cycloalkyl, A₂-substituted cycloalkyl-A₃-substituted cycloalkyl, substituted cycloalkyl-A₃-aryl, A₂-substituted cycloalkyl-A₃-aryl, substituted cycloalkyl-A₃-heteroaryl, A₂-substituted cycloalkyl-A₃-heteroaryl, substituted cycloalkyl-A₃-heterocycloalkyl, A₂-substituted cycloalkyl-A₃-heterocycloalkyl, substituted cycloalkyl-A₃-substituted aryl, A₂-substituted cycloalkyl-A₃-substituted aryl, heteroaryl-A₃-heteroaryl, A₂-heteroaryl-A₃-heteroaryl, heteroaryl-A₃-cycloalkyl, A₂-heteroaryl-A₃-cycloalkyl, heteroaryl-A₃-substituted cycloalkyl, A₂-heteroaryl-A₃-substituted cycloalkyl, heteroaryl-A₃-aryl, A₂-heteroaryl-A₃-aryl, heteroaryl-A₃-heterocycloalkyl, A₂-heteroaryl-A₃-heterocycloalkyl, heteroaryl-A₃-substituted aryl, A₂-heteroaryl-A₃-substituted aryl, heterocycloalkyl-A₃-heterocycloalkyl, A₂-heterocycloalkyl-A₃-heterocycloalkyl, heterocycloalkyl-A₃-cycloalkyl, A₂-heterocycloalkyl-A₃-cycloalkyl, heterocycloalkyl-A₃-substituted cycloalkyl, A₂-heterocycloalkyl-A₃-substituted cycloalkyl, heterocycloalkyl-A₃-aryl, A₂-heterocycloalkyl-A₃-aryl, heterocycloalkyl-A₃-substituted aryl, A₂-heterocycloalkyl-A₃-substituted aryl, heterocycloalkyl-A₃-heteroaryl, A₂-heterocycloalkyl-A₃-heteroaryl, substituted aryl-A₃-substituted aryl, A₂-substituted aryl-A₃-substituted aryl, substituted aryl-A₃-cycloalkyl, A₂-substituted aryl-A₃-cycloalkyl, substituted aryl-A₃-substituted cycloalkyl, A₂-substituted aryl-A₃-substituted cycloalkyl, substituted aryl-A₃-aryl, A₂-substituted aryl-A₃-aryl, substituted aryl-A₃-heteroaryl, A₂-substituted aryl-A₃-heteroaryl, substituted aryl-A₃-heterocycloalkyl, and A₂-substituted aryl-A₃-heterocycloalkyl;
R₆ is hydrogen, alkyl, substituted alkyl, cycloalkyl, substituted cycloalkyl, A₂-cycloalkyl, A₂-substituted cycloalkyl, aryl, substituted aryl, A₂-aryl, A₂-substituted aryl, aryl-A₃-aryl, A₂-aryl-A₃-aryl, heteroaryl, A₂-heteroaryl, heterocycloalkyl, A₂-heterocycloalkyl, aryl-A₃-cycloalkyl, A₂-aryl-A₃-cycloalkyl, aryl-A₃-heteroaryl, A₂-aryl-A₃-heteroaryl, aryl-A₃-heterocycloalkyl, A₂-aryl-A₃-heterocycloalkyl, carboxy, alkoxycarbonyl, aryloxycarbonyl, 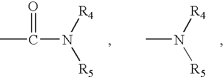
alkoxycarbonylamino, aryloxycarbonylamino, arylcarbonylamino, —N(alkyl)(alkoxycarbonyl), —N(alkyl)(aryloxycarbonyl), alkylcarbonylamino, —N(alkyl)(alkylcarbonyl), or —N(alkyl)(arylcarbonyl);
m is an integer from 1 to 5;
Y is O, S, N—R₄, N—SO₂—R₇, 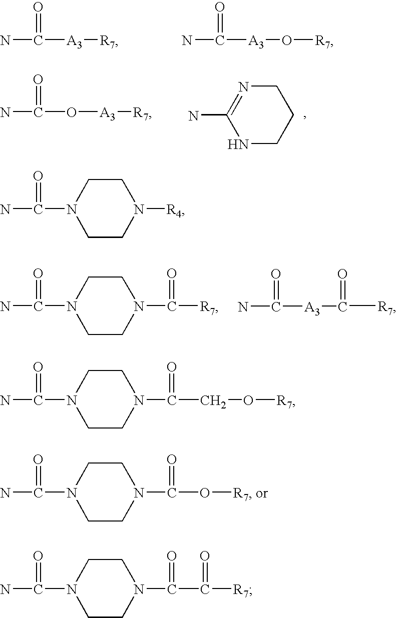
R₇ is hydrogen, alkyl, substituted alkyl, cycloalkyl, substituted cycloalkyl, A₂-cycloalkyl, A₂-substituted cycloalkyl, aryl, substituted aryl, A₂-aryl, A₂-substituted aryl, heteroaryl, A₂-heteroaryl, heterocycloalkyl, A₂-heterocycloalkyl, aryl-A₃-aryl, A₂-aryl-A₃-aryl, aryl-A₃-cycloalkyl, A₂-aryl-A₃-cycloalkyl, aryl-A₃-heteroaryl, A₂-aryl-A₃-heteroaryl, aryl-A₃-heterocycloalkyl, A₂-aryl-A₃-heterocycloalkyl, aryl-A₃-substituted aryl, A₂-aryl-A₃-substitued aryl, aryl-A₃-substituted cycloalkyl, A₂-aryl-A₃-substituted cycloalkyl, cycloalkyl-A₃-cycloalkyl, A₂-cycloalkyl-A₃-cycloalkyl, cycloalkyl-A₃-aryl, A₂-cycloalkyl-A₃-aryl, cycloalkyl-A₃-heteroaryl, A₂-cycloalkyl-A₃-heteroaryl, cycloalkyl-A₃-heterocycloalkyl, A₂-cycloalkyl-A₃-heterocycloalkyl, cycloalkyl-A₃-substituted cycloalkyl, A₂-cycloalkyl-A₃-substituted cycloalkyl, cycloalkyl-A₃-substituted aryl, A₂-cycloalkyl-A₃-substituted aryl, substituted cycloalkyl-A₃-cycloalkyl, A₂-substituted cycloalkyl-A₃-cycloalkyl, substituted cycloalkyl-A₃-substituted cycloalkyl, A₂-substituted cycloalkyl-A₃-substituted cycloalkyl, substituted cycloalkyl-A₃-aryl, A₂-substituted cycloalkyl-A₃-aryl, substituted cycloalkyl-A₃-heteroaryl, A₂-substituted cycloalkyl-A₃-heteroaryl, substituted cycloalkyl-A₃-heterocycloalkyl, A₂-substituted cycloalkyl-A₃-heterocycloalkyl, substituted cycloalkyl-A₃-substituted aryl, A₂-substituted cycloalkyl-A₃-substituted aryl, heteroaryl-A₃-heteroaryl, A₂-heteroaryl-A₃-heteroaryl, heteroaryl-A₃-cycloalkyl, A₂-heteroaryl-A₃-cycloalkyl, heteroaryl-A₃-substituted cycloalkyl, A₂-heteroaryl-A₃-substituted cycloalkyl, heteroaryl-A₃-aryl, A₂-heteroaryl-A₃-aryl, heteroaryl-A₃-heterocycloalkyl, A₂-heteroaryl-A₃-heterocycloalkyl, heteroaryl-A₃-substituted aryl, A₂-heteroaryl-A₃-substituted aryl, heterocycloalkyl-A₃-heterocycloalkyl, A₂-heterocycloalkyl-A₃-heterocycloalkyl, heterocycloalkyl-A₃-cycloalkyl, A₂-heterocycloalkyl-A₃-cycloalkyl, heterocycloalkyl-A₃-substituted cycloalkyl, A₂-heterocycloalkyl-A₃-substituted cycloalkyl, heterocycloalkyl-A₃-aryl, A₂-heterocycloalkyl-A₃-aryl, heterocycloalkyl-A₃-substituted aryl, A₂-heterocycloalkyl-A₃-substituted aryl, heterocycloalkyl-A₃-heteroaryl, A₂-heterocycloalkyl-A₃-heteroaryl, substituted aryl-A₃-substituted aryl, A₂-substituted aryl-A₃-substituted aryl, substituted aryl-A₃-cycloalkyl, A₂-substituted aryl-A₃-cycloalkyl, substituted aryl-A₃-substituted cycloalkyl, A₂-substituted aryl-A₃-substituted cycloalkyl, substituted aryl-A₃-aryl, A₂-substituted aryl-A₃-aryl, substituted aryl-A₃-heteroaryl, A₂-substituted aryl-A₃-heteroaryl, substituted aryl-A₃-heterocycloalkyl, A₂-substituted aryl-A₃-heterocycloalkyl, 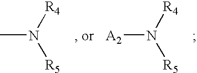
n and o are independently one or two provided that the sum of n plus o is two or three;
v and w are independently one, two, or three provided that the sum of v plus w is three, four, or five;
R₈ is hydrogen, halo, amino, —NH(lower alkyl), —N(lower alkyl)₂, nitro, alkyl, substituted alkyl, alkoxy, hydroxy, aryl, substituted aryl, A₂-aryl, A₂-substituted aryl, aryl-A₃-aryl, A₂-aryl-A₃-aryl, cycloalkyl, substituted cycloalkyl, A₂-cycloalkyl, A₂-substituted cycloalkyl, heteroaryl, A₂-heteroaryl, heterocycloalkyl, A₂-heterocycloalkyl, aryl-A₃-cycloalkyl, A₂-aryl-A₃-cycloalkyl, aryl-A₃-heteroaryl, A₂-aryl-A₃-heteroaryl, aryl-A₃-heterocycloalkyl, or A₂-aryl-A₃-heterocycloalkyl;
B₁, B₂ and B₃ are each CH, or two of B₁, B₂ and B₃ are CH and the other is N, or one of B₁, B₂ and B₃ is CH and the other two are N;
R₉ is hydrogen or lower alkyl;
R₁₀ is alkyl, substituted alkyl, alkyl-O-alkyl, alkyl-O-alkyl-O-alkyl, cycloalkyl, substituted cycloalkyl, A₂-cycloalkyl, A₂-substituted cycloalkyl, aryl, substituted aryl, A₂-aryl, A₂-substituted aryl, aryl-A₃-aryl, A₂-aryl-A₃-aryl, heteroaryl, A₂-heteroaryl, heterocycloalkyl, A₂-heterocycloalkyl, aryl-A₃-cycloalkyl, A₂-aryl-A₃-cycloalkyl, aryl-A₃-heteroaryl, A₂-aryl-A₃-heteroaryl, aryl-A₃-heterocycloalkyl or A₂-aryl-A₃-heterocycloalkyl;
R₂₁ and R₂₂ are the same or different and are independently selected from hydrogen, alkyl, substituted alkyl, cycloalkyl, substituted cycloalkyl, A₂-cycloalkyl, A₂-substituted cycloalkyl, A₂-aryl, and A₂-substituted aryl;
p is an integer from 2 to 6;
q is an integer from 1 to 6;
r is zero, 1 or 2;
s is 1 or 2;
t is 1, 2, 3 or 4;
u is 1, 2 or 3;
A₂ is an alkylene or a substituted alkylene bridge of 1 to 10 carbons, an alkenyl or substituted alkenyl bridge of 2 to 10 carbons having one or more double bonds, or an alkynyl or substituted alkynyl bridge of 2 to 10 carbons having one or more triple bonds;
A₃ is a bond, an alkylene or a substituted alkylene bridge of 1 to 10 carbons, an alkenyl or substituted alkenyl bridge of 2 to 10 carbons having one or more double bonds, an alkynyl or substituted alkynyl bridge of 2 to 10 carbons having one or more triple bonds, 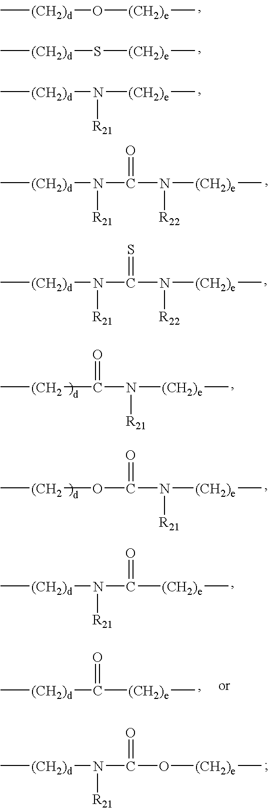
d and e are independently selected from zero and an integer from 1 to 10 provided that the sum of d plus e is no greater than 10;
and an inner salt or a pharmaceutically acceptable salt thereof, a hydrolyzable ester thereof, or a solvate thereof;

23. The method as defined in Claim 22 for treating and/or preventing asthma or allergic rhinitis.

24. The method for treating chronic asthma as defined in Claim 22 which comprises administering to a mammalian species by inhalation to the bronchioles an effective amount of said compound.
